(19) United States
(12) Patent Application Publication
(10) Pub. No.: US 2003/0093819 A1
(43) Pub. Date: May 15, 2003

(54) Methods and compositions for the diagnosis of cancer susceptibilities and defective DNA repair mechanisms and treatment thereof

(76) Inventors: Alan D. D'Andrea, Winchester, MA (US); Toshiyasu Taniguchi, Boston, MA (US); Cynthia Timmers, Columbus, OH (US); Markus Grompe, Portland, OR (US)

Correspondence Address: BROMBERG & SUNSTEIN LLP, 125 SUMMER STREET, BOSTON, MA, 02110-1618, US

(21) Appl. No.: 09/998,027

(22) Filed: Nov. 2, 2001

Related U.S. Application Data
(60) Provisional application No. 60/245,756, filed on Nov. 3, 2000.

(51) Int. Cl. A01K 67/00; C12Q 1/68; C07H 21/04; C12N 9/00; C12P 21/02; C12N 5/06

(52) U.S. Cl. 800/8; 800/10; 536/23.2; 435/6; 435/183; 435/69.1; 435/325; 435/320.1

(57) ABSTRACT

Methods and compositions for the diagnosis of cancer susceptibilities, defective DNA repair mechanisms and treatments thereof are provided. Among sequences provided here, the FANCD2 gene has been identified, mapped on the 3p chromosome, cloned into recombinant vectors, used to prepare recombinant cells and sequenced. The FANCD2 gene sequence provides probes and primers for screening patients in genetic based tests and for diagnosing Fanconi anemia and cancer. It has also been possible to target the FANCD2 gene in vivo for preparing experimental mouse models for use in screening new therapeutic agents for treating conditions involving defective DNA repair. Vectors are described for use in gene therapy. The FANCD2 polypeptide has been sequenced and has been shown to exist in two isoforms identified as FANCD2-S and the mono-ubiquinated FANCD-L form. Antibodies including polyclonal and monoclonal antibodies have been prepared that distinguish the two isoforms and have been used in diagnostic tests to determine whether a subject has an intact FA pathway. The FANCD2 has been localized to the nucleus and is associated with BRCA 1 foci.

### CROSS REFERENCE TO RELATED APPLICATIONS

This application gains priority from provisional application 60/245,756 filed Nov. 3, 2000, the application being incorporated by reference herein.

The work described herein was supported by the National Institute of Health, NIH Grant vNo. Health grants RO1HL52725-04, RO1 DK43889-09, 1PO1HL48546, and PO1HL54785-04. The US Government has certain rights to the claimed invention

### TECHNICAL FIELD AND BACKGROUND ART

The present invention relates to the diagnosis of cancer susceptibilities in subjects having a defect in the FANCD2 gene and the determination of suitable treatment protocols for those subjects who have developed cancer. Animal models with defects in the FANCD2 gene can be used to screen for therapeutic agents.

Fanconi Anemia (FA) is an autosomal recessive cancer susceptibility syndrome characterized by birth defects, bone marrow failure and cancer predisposition. Cells from FA patients display a characteristic hypersensitivity to agents that produce interstrand DNA crosslinks such as mitomycin C or diepoxybutane. FA patients develop several types of cancers including acute myeloid leukemias and cancers of the skin, gastrointestinal; and gynecological systems. The skin and gastrointestinal tumors are usually squamous cell carcinomas. At least 20% of patients with FA develop cancers. The average age of patients who develop cancer is 15 years for leukemia, 16 years for liver tumors and 23 years for other tumors. (D'Andrea et al., Blood, (1997) Vol. 90, pp. 1725, Garcia-Higuera et al. Curr. Opin. Hematol, (1999) Vol 2, pp. 83-88 and Heijna et al. Am. J. Hum. Genet. Vol 66, pp 1540-1551)

FA is genetically heterogeneous. Somatic cell fusion studies have identified at least seven distinct complementation groups (Joenje et al., (1997) Am. J. Hum. Genet., Vol. 61, pp. 940-944 and Joenje et al., (2000) Am. J. Hum. Genet, Vol. 67, pp. 759-762). This observation has resulted in the hypothesis that the FA genes define a multicomponent pathway involved in cellular responses to DNA cross-links. Five of the FA genes (FANCA, FANCC, FANCE, FANCF and FANCG) have been cloned and the FANCA, FANCC and FANCG proteins have been shown to form a molecular complex with primarily nuclear localization. FANCC also localizes in the cytoplasm. Different FA proteins have few or no known sequence motifs with no strong homologs of the FANCA, FANCC, FANCE, FANCF, and FANCG proteins in non-vertebrate species. FANCF has weak homology of unknown significance to an E. Coli RNA binding protein. The two most frequent complementation groups are FA—A and FA—C which together account for 75%-80% of FA patients. Multiple mutations have been recognized in the FANCA gene that span 80 kb and consists of at least 43 exons. FANCC has been found to have 14 exons and spans approximately 80 kb. A number of mutations in the FANCC gene have been identified which are correlated with FA of differing degrees of severity. FA—D has been identified as a distinct but rare complementation group. Although FA—D patients are phenotypically distinguishable from patients from other subtypes, the FA protein complex assembles normally in FA—D cells (Yamashita et al., (1998) P.N.A.S., Vol. 95, pp.13085-13090)

The cloned FA proteins encode orphan proteins with no sequence similarity to each other or to other proteins in GenBank and no functional domains are apparent in the protein sequence. Little is known regarding the cellular or biochemical function of these proteins.

Diagnosis of FA is complicated by the wide variability in FA patient phenotype. Further confounding diagnosis, approximately 33% of patients with FA have no obvious congenital abnormalities. Moreover, existing diagnostic tests do not differentiate FA carriers from the general population. The problems associated with diagnosis are described in D'Andrea et al., (1997). Many cellular phenotypes have been reported in FA cells but the most consistent is hypersensitivity to bifunctional alkylating agents such as mitomycin C or diepoxybutane. These agents produce interstrand DNA cross-links (an important class of DNA damage).

Diagnosing cancer susceptibility is complicated because of the large number of regulatory genes and biochemical pathways that have been implicated in the formation of cancers. Different cancers depending on how they arise and the genetic lesions involved may determine how a subject responds to any particular therapeutic treatments. Genetic lesions that are associated with defective repair mechanisms may give rise to defective cell division and apoptosis which in turn may increase a patient's susceptibility to cancer. FA is a disease condition in which multiple pathological outcomes are associated with defective repair mechanisms in addition to cancer susceptibility

An understanding of the molecular genetics and cell biology of Fanconi Anemia pathway can provide insights into prognosis, diagnosis and treatment of particular classes of cancers and conditions relating to defects in DNA repair mechanisms that arise in non-FA patients as well as FA patients

### SUMMARY OF THE INVENTION

In a first embodiment of the invention there is provided an isolated nucleic acid molecule that includes a polynucleotide selected from (a) a nucleotide sequence encoding a polypeptide having an aminoacid sequence as shown in SEQ ID NO: 4 (b) a nucleotide sequence at least 90% identical to the polynucleotide of (b); (c) a nucleotide sequence complementary to the polynucleotide of (b); (d) a nucleotide sequence at least 90% identical to the nucleotide sequence shown in SEQ ID No: 5-8, 187-188; and (e) a nucleotide sequence complementary to the nucleotide sequence of (d). The polynucleotide may be an RNA molecule or a DNA molecule, such as a cDNA.

In another embodiment of the invention, an isolated nucleic acid molecule is provided that consists essentially of a nucleotide sequence encoding a polypeptide having an amino acid sequence sufficiently similar to that of SEQ ID No: 4 to retain the biological property of conversion from a short form to a long form of FANCD2 in the nucleus of a cell for facilitating DNA repair. Alternately, the isolated nucleic acid molecule consists essentially of a polynucleotide having a nucleotide sequence at least 90% identical to SEQ ID NO: 9-191 or complementary to a nucleotide sequence that is at least 90% identical to SEQ ID NO: 9-191.

In an embodiment, a method is provided for making a recombinant vector that includes inserting any of the isolated nucleic acid molecules described above into a vector. A recombinant vector product may be made by this method and the vector may be introduced to form a recombinant host cell into a host cell.

In an embodiment of the invention, a method is provided for making an FA—D2 cell line, that includes (a) obtaining cells from a subject having a biallelic mutation in a complementation group associated with FA—D2; and (b) infecting the cells with a transforming virus to make the FA—D2 cell line where the cells may be selected from fibroblasts and lymphocytes and the transforming virus selected from Epstein Barr virus and retrovirus. The FA—D2 cell line may be characterized by determining the presence of a defective FANDC2 in the cell line for example by performing a diagnostic assay selected from (i) a Western blot or nuclear immunofluorescence using an antibody specific for FANCD2 and (ii) a DNA hybridization assay.

In an embodiment of the invention, a recombinant method is provided for producing a polypeptide, that includes culturing a recombinant host cell wherein the host cell includes any of the isolated nucleic acid molecules described above.

In an embodiment of the invention, an isolated polypeptide, including an aminoacid sequence selected from (a) SEQ ID NO: 4; (b) an aminoacid sequence at least 90% identical to (a); (c)

an aminoacid sequence which is encoded by a polynucleotide having a nucleotide sequence which is at least 90% identical to at least one of SEQ ID NO: 5-8, 187-188; (d) an aminoacid sequence which is encoded by a polynucleotide having a nucleotide sequence which is at least 90% identical to a complementary sequence to at least one of SEQ ID NO: 5-8, 187-188; and (e) a polypeptide fragment of (a) -(d) wherein the fragment is at least 50 aminoacids in length.

The isolated polypeptide may be encoded by a DNA having a mutation selected from nt 376 A to G, nt 3707 G to A, nt9O4C to T and nt 958C to T. Alternatively, the polypeptide may be characterized by a polymorphism in DNA encoding the polypeptide, the polymorphism being selected from nt 1122A to G, nt 1440T to C, nt1509C to T, nt2141C to T, nt2259T to C, nt4098T to G, nt4453G to A. Alternatively, the polypeptide may be characterized by a mutation at aminoacid 222 or aminoacid 561.

In an embodiment of the invention, an antibody preparation is described having a binding specificity for a FANCD2 protein where the antibody may be a monoclonal antibody or a polyclonal antibody and wherein the FANCD2 may be FANCD2-S or FANCD2-L.

In an embodiment of the invention, a diagnostic method is provided for measuring FANCD2 isoforms in a biological sample where the method includes (a) exposing the sample to a first antibody for forming a first complex with FANCD2-L and optionally a second antibody for forming a second complex with FANCD2-S; and (b) detecting with a marker, the amount of the first complex and the second complex in the sample. The sample may be intact cells or lyzed cells in a lysate. The biological sample may be from a human subject with a susceptibility to cancer or having the initial stages of cancer. The sample may be from a cancer in a human subject, wherein the cancer is selected from melanoma, leukemia, astocytoma, glioblastoma, lymphoma, glioma, Hodgkins lymphoma, chronic lymphocyte leukemia and cancer of the pancreas, breast, thyroid, ovary, uterus, testis, pituitary, kidney, stomach, esophagus and rectum. The biological sample may be from a human fetus or from an adult human and may be derived from any of a blood sample, a biopsy sample of tissue from the subject and a cell line. The biological sample may be derived from heart, brain, placenta, liver, skeletal muscle, kidney, pancreas, spleen, thymus, prostate, testis, uterus, small intestine, colon, peripheral blood or lymphocytes. The marker may be a fluorescent marker, the fluorescent marker optionally conjugated to the FANCD2-L antibody, a chemiluminescent marker optionally conjugated to the FANCD2-L antibody and may bind the first and the second complex to a third antibody conjugated to a substrate. Where the sample is a lysate, it may be subjected to a separation procedure to separate FANCD2 isoforms and the seperated isoforms may be identified by determining binding to the first or the second FANCD2 antibody.

In an embodiment of the invention, a diagnostic test is provided for identifying a defect in the Fanconi Anemia pathway in a cell population from a subject, that includes selecting an antibody to FANCD2 protein and determining whether the amount of an FAND2-L isoform is reduced in the cell population compared with amounts, in a wild type cell population; such that if the amount of the FANCD2-L protein is reduced, then determining whether an amount of any of FANCA, FANCB, FANCC, FANCD I, FANCE, FANCF or FANCG protein is altered in the cell population compared with the wild type so as to identify the defect in the Fanconi Anemia pathway in the cell population. In one example, the amount of an isoform relies on a separation of the FANCD2-L and FANCD2-S isoforms where the separation may be achieved by gel electrophoresis or by a migration binding banded test strip.

In an embodiment of the invention, a screening assay for identifying a therapeutic agent, is provided that includes selecting a cell population in which FAND2-L is made in reduced amounts; exposing the cell population to individual members of a library of candidate therapeutic molecules; and identifying those individual member molecules that cause the amount of FANCD2-L to be increased in the cell population. In one example, the cell population is an in vitro cell population. In another example, the cell population is an in vivo cell population, the in vivo population being within an experimental animal, the experimental animal having a mutant FANCD2 gene. In a further example, the experimental animal is a knock-out mouse in which the mouse FAND2 gene has been replaced by a human mutant FANCD2 gene. In another example, a chemical carcinogen is added to the cell population in which FANCD2 is made in reduced amounts, to determine if any member molecules can cause the amount of FANCD2-L to be increased so as to protect the cells form the harmful effects of the chemical carcinogen.

In an embodiment of the invention, an experimental animal model is provided in which the animal FANCD2 gene has been removed and optionally replaced by any of the nucleic acid molecules described above

In an embodiment of the invention, a method is provided for identifying in a cell sample from a subject, a mutant FANCD2 nucleotide sequence in a suspected mutant FANCD2 allele which comprises comparing the nucleotide sequence of the suspected mutant FANCD2 allele with the wild type FANCD2 nucleotide sequence wherein a difference between the suspected mutant and the wild type sequence identifies a mutant FANCD2 nucleotide sequence in the cell sample. In one example, the suspected mutant allele is a germline allele. In another example, identification of a mutant FANCD2 nucleotide sequence is diagnostic for a predisposition for a cancer in the subject or for an increased risk of the subject bearing an offspring with Fanconi Anemia. In another example, the suspected mutant allele is a somatic allele in a tumor type and identifying a mutant FANCD2 nucleotide sequence is diagnostic for the tumor type. In another example, the nucleotide sequence of the wild type and the suspected mutant FANCD2 nucleotide sequence is selected from a gene, a mRNA and a cDNA made from a mRNA. In another example, comparing the polynucleotide sequence of the suspected mutant FANCD2 allele with the wild type FANCD2 polynucleotide sequence, further includes selecting a FANCD2 probe which specifically hybridizes to the mutant FANCD2 nucleotide sequence, and detecting the presence of the mutant sequence by hybridization with the probe. In another example, comparing the polynucleotide sequence of the suspected mutant FANCD2 allele with the wild type FANCD2 polynucleotide sequence, further comprises amplifying all or part of the FANCD2 gene using a set of primers specific for wild type FANCD2 DNA to produce amplified FANCD2 DNA and sequencing the FANCD2 DNA so as to identify the mutant sequence. In another example, where the mutant FANCD2 nucleotide sequence is a germline alteration in the FANCD2 allele of the human subject, the alteration is selected from the alterations set forth in Table 3 and where the mutant FANCD2 nucleotide sequence is a somatic alteration in the FANCD2 allele of the human subject, the alteration is selected from the alterations set forth in Table 3.

In an embodiment of the invention, a method is provided for diagnosing a susceptibility to cancer in a subject which comprises comparing the germline sequence of the FANCD2 gene or the sequence of its MRNA in a tissue sample from the subject with the germline sequence of the FANCD2 gene or the sequence of its niRNA wherein an alteration in the germline sequence of the FANCD2 gene or the sequence of its mRNA of the subject indicates the susceptibility to the cancer. An alteration may be detected in a regulatory region of the FANCD2 gene. An alteration in the germline sequence may be determined by an assay selected from the group consisting of (a) observing shifts in electrophoretic mobility of single-stranded DNA on non-denaturing polyacrylamide gels, (b) hybridizing a FANCD2 gene probe to genomic DNA isolated from the tissue sample, (c) hybridizing an allele-specific probe to genomic DNA of the tissue sample, (d) amplifying all or part of the FANCD2 gene from the tissue sample to produce an amplified sequence and sequencing the amplified sequence, (e) amplifying all or part of the FANCD2 gene from the tissue sample using primers for a specific FANCD2 mutant allele, (f) molecularly cloning all or part of the FANCD2 gene from the tissue sample to produce a cloned sequence and sequencing the cloned sequence, (g) identifying a mismatch between (i) a FANCD2 gene or a FANCD2 mRNA isolated from the tissue sample, and (ii) a nucleic acid probe complementary to the human wild-type FANCD2 gene sequence, when molecules (i) and (ii) are hybridized to each other to form a duplex, (h) amplification of FANCD2 gene sequences in the tissue sample and hybridization of the amplified sequences to nucleic acid probes which comprise wild-type FANCD2 gene sequences, (i) amplification of FANCD2 gene sequences in the tissue sample and hybridization of the amplified sequences to nucleic acid probes which comprise mutant FANCD2 gene sequences, (j) screening for a deletion mutation in the tissue sample, (k) screening for a point mutation in the tissue sample, (1) screening for an insertion mutation in the tissue sample, and (m) in situ hybridization of the FANCD2 gene of said tissue sample with nucleic acid probes which comprise the FANCD2 gene.

In an embodiment of the invention, a method is provided for diagnosing a susceptibility for cancer in a subject, includes:(a) accessing genetic material from the subject so as to determine defective DNA repair; (b) determining the presence of mutations in a set of genes, the set comprising FAND2 and at least one of FANCA, FANCB, FANCC, FANCDl, FANCDE, FANDF, FANDG, BRACAI and ATM; and (c) diagnosing susceptibility for cancer from the presence of mutations in the set of genes.

In an embodiment of the invention, a method is provided for detecting a mutation in a neoplastic lesion at the FANCD2 gene in a human subject which includes: comparing the sequence of the FANCD2 gene or the sequence of its mRNA in a tissue sample from a lesion of the subject with the sequence of the wild-type FANCD2 gene or the sequence of its mRNA, wherein an alteration in the sequence of the FANCD2 gene or the sequence of its MRNA of the subject indicates a mutation at the FANCD2 gene of the neoplastic lesion. A therapeutic protocol may be provided for treating the neoplastic lesion according to the mutation at the FANCD2 gene of the neoplastic lesion.

In an embodiment of the invention, a method is provided for confirming the lack of a FANCD2 mutation in a neoplastic lesion from a human subject which comprises comparing the sequence of the FANCD2 gene or the sequence of its mRNA in a tissue sample from a lesion of said subject with the sequence of the wild-type FANCD2 gene or the sequence of its RNA, wherein the presence of the wild-type sequence in the tissue sample indicates the lack of a mutation at the FANCD2 gene.

In an embodiment of the invention, a method is provided for determining a therapeutic protocol for a subject having a cancer, that includes (a) determining if a deficiency in FANCD2-L occurs in a cell sample from the subject by measuring FANCD2 isoforms using specific antibodies (b) if a deficiency is detected in (a), then determining whether the deficiency is a result of genetic defect in non-cancer cells; and (c) if (b) is positive, reducing the use of a therapeutic protocol that causes increased DNA damage so as to protect normal tissue in the subject and if (b) is negative, and the deficiency is contained within a genetic defect in cancer cells only, then increasing the use of a therapeutic protocol that causes increased DNA damage so as to adversely affect the cancer cells.

In an embodiment of the invention, a method of treating a FA pathway defect in a cell target is provided that includes: administering an effective amount of FANCD2 protein or an exogenous nucleic acid to the target. The FA pathway defect may be a defective FANCD2 gene and the exogenous nucleic acid vector may further include introducing a vector according to those described above. The vector may be selected from a mutant herpesvirus, a E1/E4 deleted recombinant adenovirus, a mutant retrovirus, the viral vector being defective in respect of a viral gene essential for production of infectious new virus particles. The vector may be contained in a lipid micelle.

In an embodiment of the invention, a method is provided for treating a patient with a defective FANCD2 gene, that includes providing a polypeptide described in SEQ ID No: 4, for functionally correcting a defect arising from a condition arising from the defective FANCD2 gene.

In an embodiment of the invention, a cell based assay for detecting a FA pathway defect is provided that includes obtaining a cell sample from a subject; exposing the cell sample to DNA damaging agents; and detecting whether FANCD2-L is upregulated, the absence of upregulation being indicative of the FA pathway defect. In the cell-based assay, amounts of FANCD2 may be measured by an analysis technique selected from: immunoblotting for detecting nuclear foci; Western blots to detect amounts of FANCD2 isoforms and quantifying mRNA by hybridising with DNA probes.

In an embodiment of the invention, a kit is provided for use in detecting a cancer cell in a biological sample, that includes (a) primer pair which binds under high stringency conditions to a sequence in the FANCD2 gene, the primer pair being selected to specifically amplify an altered nucleic acid sequence described in Table 7; and containers for each of the primers.

### BRIEF DESCRIPTION OF THE FIGURES

The foregoing features of the invention will be more readily understood by reference to the following detailed description, taken with reference to the accompanying drawings, in which:

FIG. 1A provides a Western blot demonstrating that the Fanconi Anemia protein complex is required for the monoubiquitination of FANCD2. Normal (WT) cells (lane 1) express two isoforms of the FANCD2 protein, a low molecular weight isoform (FANCD2-S) (155 kD) and a high molecular weight isoform (FANCD2-L) (162 kD). Lanes 3, 7, 9, 11 show that FA cell lines derived from type A, C, G, and F patients only express the FANCD2-S isoform. Lanes 4, 8, 10, 12 show the restoration of the high molecular weight isoform FANCD2-L following transfection of cell lines with corresponding FAcDNA.

FIG. 1B shows a Western blot obtained after HeLa cells were transfected with a cDNA encoding HA-ubiquitin. After transfection, cells were treated with the indicated dose of mitomycin C (MMC). Cellular proteins were immunoprecipitated with a polyclonal antibody (E35) to FANCD2, as indicated. FANCD2 was immunoprecipitated, and immune complexes were blotted with anti-FANCD2 or anti-HA monoclonal antibody.

FIG. 1C shows a Western blot obtained after HeLa cells were transfected with a cDNA encoding HA-ubiquitin. After transfection, cells were treated with the indicated dose of ionizing radiation (IR). FANCD2 was immunoprecipitated, and immune complexes were blotted with anti-FANCD2 or anti-HA monoclonal antibody.

FIG. 1D shows a Western blot obtained after FA—G fibroblast line (FAG326SV) or corrected cells (FAG326SV plus FANCG CDNA) were transfected with the HA-Ub CDNA, FANCD2 was immunoprecipitated, and immune complexes were blotted with anti-FANCD2 or anti-HA antisera.

FIG. 1E shows a Western blot obtained after treatment of Hela cells with lmM hydroxyurea for 24 hours. HeLa cell lysates were extracted and incubated at the indicated temperature for the indicated time period with or without 2.5 μM ubiquitin aldehyde. The FANCD2 protein was detected by immunoblot with monoclonal anti-FANCD2 (FI17).

FIG. 2 demonstrates that the Fanconi Anemia pathway is required for the formation of FANCD2 nuclear foci. Top panel shows anti-FANCD2 im-munoblots of SV40 transformed fibroblasts prepared as whole cell extracts. Panels a-h show immunofluorescence with the affinity-purified anti-FANCD2 antiserum. The uncorrected (mutant, M) FA fibroblasts were FA—A (GM6914), FA—G (FAG326SV), FA—C (PD426), and FA—D (PD20F). The FA—A, FA—G, and FA—C fibroblasts were functionally complemented with the corresponding FA CDNA. The FA—D cells were complemented with neomycin-tagged human chromosome 3p (Whitney and al, 1995).

FIG. 3 shows the cell cycle dependent expression of the two isoforms of the FANCD2 protein. (a) HeLa cells, SV40 transformed fibroblasts from an FA—A patient (GM6914), and GM6914 cells corrected with FANCA cDNA were synchronized by the double thymidine block method. Cells corresponding to the indicated phase of the cell cycle were lysed, and processed for FANCD2 immunoblotting (b) Synchrony by nocodazole block (c) Synchrony by mimosine block (d) HeLa cells were synchronized in the cell cycle using nocodozole or (e) mimosine, and cells corresponding to the indicated phase of the cell cycle were immunostained with the anti-FANCD2 antibody and analyzed by immunofluorescence.

FIG. 4. shows the formation of activated FANCD2 nuclear foci following cellular exposure to MMC, Ionizing Radiation, or Ultraviolet Light. Exponentially-growing HeLa cells were either untreated or exposed to the indicated DNA damaging agents, (a) Mitomycin C (MMC), (b) γ-irradiation (IR), or (c) Ultraviolet Light (UV), and processed for FANCD2 immunoblotting or FANCD2 immunostaining. (a) Cells were continuosly exposed to 40 ng/ml MMC for 0-72 hours as indicated, or treated for 24 hours and fixed for immunofluorescence. (b) and (c) Cells were exposed to γ-irradiation (10 Gy, B) or UV light (60 J/m² C) and collected after the indicated time (upper panels) or irradiated with the indicated doses and harvested one hour later (lower panels). For immunofluorescence analysis cells were fixed 8 hours after treatment (B, 10 Gy, C, 60 J/m²). (d) The indicated EBV-transformed lymphoblast lines from a normal individual (PD7) or from various Fanconi Anemia patients were either treated with 40 ng/ml of Mitomycin C continuously (lanes 1-21) or exposed to 15 Gy of γ-irradiation (lanes 22-33) and processed for FANCD2 immunoblotting. The upregulation of FANCD-L after MMC or IR treatment was seen in PD7 (lanes 2-5) and in the corrected FA—A cells (lanes 28-33), but was not observed in any of the mutant Fanconi Anemia cell lines. Similarly, IR-induced FANCD2 nuclear foci were not detected in FA fibroblasts (FA—G +IR) but were restored after functional complementation (FA—G+FANCG).

FIG. 5 shows co-localization of activated FANCD2 and BRCA1 in Discrete Nuclear Foci following DNA damage. HeLa cells were untreated or exposed to Ionizing Radiation (10 Gy) as indicated, and fixed 8 hours later. (a) Cells were double-stained with the D-9 monoclonal anti-BRCAl antibody (green, panels a, d, g, h) and the rabbit polyclonal anti-FANCD2 antibody (red, panels b, e, h, k), and stained cells were analyzed by immunofluorescence. Where green and red signals overlap (Merge, panels c, f, i, 1) a yellow pattern is seen, indicating colocalization of BRCA1 and FANCD2. (b) Co-immunoprecipitation of FANCD2 and BRCA 1. HeLa cells were untreated (−IR) or exposed to 15 Gy of γγ irradiation (+IR) and collected 12 hours later. Cell lysates were prepared, and cellular proteins were immunoprecipitated with either the monoclonal FANCD2 antibody (FI-17, lanes 9-10), or any one of three independently-derived monoclonal antibodies to human BRCA1 (lanes 3-8): D-9 (Santa Cruz), Ab-1 and Ab-3 (Oncogene Research Products). The same amount of purified mouse IgG (Sigma) was used in control samples (lanes 1-2). Immune complexes were resolved by SDS-PAGE and were immunoblotted with anti-FANCD2 or anti-BRCA1 antisera. The FANCD-L isoform preferentially coimmunoprecipitated with BRCA1.

FIG. 6 shows the co-localization of activated FANCD2 and BRCA1 in discrete nuclear foci during S phase. (a) HeLa cells were synchronized in late GI with mimosine and released into S phase. S phase cells were double-stained with the monoclonal anti-BRCA1 antibody (green, panels a, d) and the rabbit polyclonal anti-FANCD2 antibody (red, panels b, e), and stained cells were analyzed by immunofluorescence. Where green and red signals overlap (merge, panels c, f), a yellow pattern is seen, indicating co-localization of BRCA1 and FANCD2. (b) HeLa cells synchronized in S phase were either untreated (a, b, k, 1) or exposed to IR (50 Gy, panels c, d, m, n), MMC (20 μg/ml, panels e, f, o, p), or UV (100 j/m2, panels g, h, q, r) as indicated and fixed 1 hour later. Cells were subsequently immunostained with an antibody specific for FANCD2 or BRCA1.

FIG. 7 shows that FANCD2 forms foci on synaptonemal complexes that can co-localize with BRCA1 during meiosis I in mouse spermatocytes. (a) Anti-SCP3 (white) and anti-FANCD2 (red) staining of synaptonemal complexes in a late pachytene mouse nucleus. (b) SCP3 staining of late pachytene chromosomes. (c) Staining of this spread with preimmune serum for the anti-FANCD2 E35 antibody. (d) Anti-SCP3 staining of synaptonemal complexes in a mouse diplotene nucleus. (e) Costaining of this spread with E35 anti-FANCD2 antibody. Note staining of both the unpaired sex chromosomes and the telomeres of the autosomes with anti-FANCD2. (f) Costaining of this spread with anti-BRCA1 antibody. The sex chromosomes are preferentially stained. (g) Anti-FANCD2 staining of late pachytene sex chromosome synaptonemal complexes. (h) Anti-BRCA1 staining of the same complexes. (i) Anti-FANCD2 (red) and anti-BRCA1 (green) co-staining (co-localization reflected by yellow areas).

FIG. 8 provides a schematic interaction of the FA proteins in a cellular pathway. The FA proteins (A, C, and G) bind in a functional nuclear complex. Upon activation of this complex, by either S phase entry or DNA damage, this complex enzymatically modifies (monoubiquitinates) the D protein. According to this model, the activated D protein is subsequently targeted to nuclear foci where it interacts with the BRCA1 protein and other proteins involved in DNA repair.

FIG. 9 shows a Northern blot of cells from heart, brain, placenta, liver, skeletal muscle, kidney, pancreas, spleen, thymus, prostate, testis, uterus, small intestine, colon and peripheral blood lymphocytes from a human adult and brain, lung, liver and kidney from a human fetus probed with a full-length FANCD2 cDNA and exposed for 24 hours.

FIG. 10 shows allele specific assays for mutation analysis of 2 FANCD2 families where the family pedigrees (a, d) and panels b, c, e and f are vertically aligned such that the corresponding mutation analysis is below the individual in question. Panels a-c depict the PD20 and panels d-f the VU008 family. Panels b and e show the segregation of the maternal mutations as detected by the creation of a new MspI site (PD20) or DdeI site (VU008). The paternally inherited mutations in both families were detected with allele specific oligonucleotide hybridization (panels c and f).

FIG. 11 shows a Western blot analysis of the FANCD2 protein in human Fanconi Anemia cell lines. Whole cell lysates were generated from the indicated fibroblast and lymphoblast lines. Protein lysates (70 μg) were probed directly by immunoblotting with the anti-FANCD2 antiserum. The FANCD2 proteins (155 kD and 162 kD) are indicated by arrows. Other bands in the immunoblot are non-specific. (a) Cell lines tested included wild-type cells (lanes 1,7), PD20 Fibroblasts (lane 2), PD20 lymphoblasts (lane 4), revertant MMC-resistant PD20 lymphoblasts (lane 5,6), and chromosome 3p complemented PD20 fibroblasts (lane 3). Several other FA group D cell lines were analyzed including HSC62 (lane 8) and VUOO8 (lane 9). FA—A cells were HSC72 (lane 10), FA—C cells were PD4 (lane 11), and FA—G cells were EUFA316 (lane 12). (b) Identification of a third FANCD2 patient. FANCD2 protein was readily detectable in wild-type and FA group G cells but not in PD733 cells. (c) Specificity of the antibody. PD20i cells transduced with a retroviral FANCD2 expression vector displayed both isoforms of the FANCD2 protein (lane 4) in contrast to empty vector controls (lane 3) and untransfected PD20i cells (lane 2). In wild-type cells the endogenous FANCD2 protein (two isoforms) was also immunoreactive with the antibody (lane 1).

FIG. 12 shows functional complementation of FA—D2 cells with the cloned FANCD2 cDNA. The SV40-transformed FA—D2 fibroblast line, PD20i, was transduced with pMMP-puro (PD20+vector) or pMMP-FANCD2 (PD20+FANCD2wt). Puromycin-selected cells were subjected to MMC sensitivity analysis. Cells analyzed were the parental PD20F cells (Δ), PD20 corrected with human chromosome 3p (o), and PD20 cells transduced with either pMMP-puro ( ) or pMMP-FANCD2(wt)-puro (♦).

FIG. 13 shows a molecular basis for the reversion of PD20 Lymphoblasts. (a) PCR primers to exons 5 and 6 were used to amplify cDNA. Control samples (right lane) yielded a single band of 114 bp, whereas PD20 cDNA (left lane) showed 2 bands, the larger reflecting the insertion of 13 bp of intronic sequence into the maternal allele. Reverted, MMC resistant lymphoblasts (middle lane) from PD20 revealed a third, in-frame splice variant of 114+36 bp (b) Schematic representation of splicing at the FANCD2 exon 5/intron 5 boundary. In wild-type cDNA 100% of splice events occur at the proper exon/intron boundary, whereas the maternal A−>G mutation (indicated by arrow) leads to aberrant splicing, also in 100%. In the reverted cells all cDNAs with the maternal mutation also had a second sequence change (fat arrow) and showed a mixed splicing pattern with insertion of either 13 bp (˜40% of mRNA) or 36 bp (˜60% of mRNA).

FIG. 14 shows an FANCD2 Western blot of cancer cell lines derived from patients with ovarian cancer.

FIG. 15 shows a sequence listing for amino acid sequence of human FANCD2 and alignment with fly and plant homologues using the BEAUTY algorithm (Worley, et al., 1995, Genome Res.Vol. 5, pp.173-184). (SEQ. ID. NO. 1-3) Black boxes indicate amino acid identity and gray similarity. The best alignment scores were observed with hypothetical proteins in D melanogaster (p=8.4×10⁻⁵⁸, accession number AAF55806) and A thaliana (p=9.4×10⁻⁴⁵, accession number B71413).

FIG. 16 is the FANCD cDNA sequence ˜63 to 5127 nucleotides (SEQ ID NO: 5) and polypeptide encoded by this sequence from amino acid 1 to 1472 (SEQ ID NO: 4).

FIG. 17 is the nucleotide sequence for FANCD-S.ORF (SEQ ID NO: 187) compared with FANCD cDNA (SEQ ID NO: 188).

FIG. 18 is the nucleotide sequence for human FANCD2-L (SEQ ID NO: 6).

FIG. 19 is the nucleotide sequence for human FANCD2-S (SEQ ID NO: 7).

FIG. 20 is the nucleotide sequence for mouse FANCD2 (SEQ ID NO: 8).

### DETAILED DESCRIPTION OF SPECIFIC EMBODIMENTS

Definitions. As used in this description and the accompanying claims, the following terms shall have the meanings indicated, unless the context otherwise requires:

“FANCD2-L therapeutic agent” shall mean any of a protein isoform, and includes a peptide, a peptide derivative, analogue or isomer of the FANCD2-L protein and further include any of a small molecule derivative, analog, isomer or agonist that is functionally equivalent to FANCD2-L. Also included in the definition is a nucleic acid encoding FANCD2 which may be a full length or partial length gene sequence or cDNA or may be a gene activating nucleic acid or a nucleic acid binding molecule including an aptamer of antisense molecule which may act to modulate gene expression.

“Nucleic acid encoding FANCD-2” shall include the complete cDNA or genomic sequence of FANCD2 or portions thereof for expressing FANCD2-L protein as defined above. The nucleic acid may further be included in a nucleic acid carrier or vector and includes nucleic acid that has been suitably modified for effective delivery to the target site.

“Stringent conditions of hybridization” will generally include temperatures in excess of 30° C., typically in excess of 37° C., and preferably in excess of 45° C. Stringent salt conditions will ordinarily be less than 1000 mM, typically less than 500 mM, and preferably less than 200 mM.

“Substantial homology or similianity” for a nucleic acid is when a nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80.

“Antibodies” includes polyclonal and/or monoclonal antibodies and fragments thereof including single chain antibodies and including single chain antibodies and Fab fragments, and immunologic binding equivalents thereof, which have a binding specificity sufficient to differentiate isoforms of a protein. These antibodies will be useful in assays as well as pharmaceuticals.

“Isolated” is used to describe a protein, polypeptide or nucleic acid which has been separated from components which accompany it in its natural state. An “isolated” protein or nucleic acid is substantially pure when at least about 60 to 75% of a sample exhibits a single aminoacid or nucleotide sequence.

“Regulatory sequences” refers to those sequences normally within 100 kb of the coding region of a locus, but they may also be more distant from the coding region, which affect the expression of the gene (including transcription of the gene, and translation, splicing, stability or the like of the messenger RNA).

“Polynucleotide” includes RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), and modified linkages (e.g., alpha anomeric nucleic acids, etc.). Also included are synthetic molecules that mimic nucleic acids in their ability to bind to a designated sequence via hydrogen bonding and other chemical interactions.

“Mutation” is a change in nucleotide sequence within a gene, or outside the gene in a regulatory sequence compared to wild type. The change may be a deletion, substitution, point mutation, mutation of multiple nucleotides, transposition, inversion, frame shift, nonsense mutation or other forms of abberration that differentiate the nucleic acid or protein sequence from that of a normally expressed gene in a functional cell where expression and functionality are within the normally occurring range.

“Subject” refers to an animal including mammal, including human.

“Wild type FANCD2” refers to a gene that encodes a protein or an expressed protein capable of being monoubquinated to form FANCD2-L from FANCD-S within a cell.

We have found that some Fanconi Anemia has similarities with a group of syndromes including ataxia telangiectasia (AT), Xeroderma pigmentosum (XP), Cockayne syndrome (CS), Bloom's syndrome, myelodysplastic syndrome, aplastic anemia, cancer susceptibility syndromes and HNPCC (see Table 2). These syndromes have an underlying defect in DNA repair and are associated with defects in maintenance of chromosomal integrity. Defects in pathways associated with DNA repair and maintenance of chromosomal integrity result in genornic instability, and cellular sensitivity to DNA damaging agents such as bifunctional alkylating agents that cause intrastrand crosslinking. Moreover, deficiencies in DNA repair mechanisms appear to substantially increase the probability of initiating a range of cancers through genetic rearrangements. This observation is pertinent with regard to the clinical use of DNA cross-linking drugs including mitomycin C, cisplatin, cyclophosphamide, psoralen and UVA irradiation.

Although Fanconi Anemia is a rare disease, the pleiotropic effects of FA indicate the importance of the wild type function of FA proteins in the pathway for diverse cellular processes including genome stability, apoptosis, cell cycle control and resistance to DNA crosslinks. The cellular abnormalities in FA include sensitivity to cross-linking agents, prolongation of G2 phase of cell cycle, sensitivity to oxygen including poor growth at ambient O₂, overproduction of O₂ radicals, deficient O₂ radical defense, deficiency in superoxide dismutase; sensitivity to ionizing radiation (G2 specific); overproduction of tumor necrosis factor, direct defects in DNA repair including accumulations of DNA adducts, and defects in repair of DNA cross-links, genomic instability including spontaneous chromosome breakage, and hypermutability by deletion mechanism, increased aptosis, defective p53 induction, intrinsic stem cell defect, including decreased colony growth in vitro; and decreased gonadal stem cell survival.

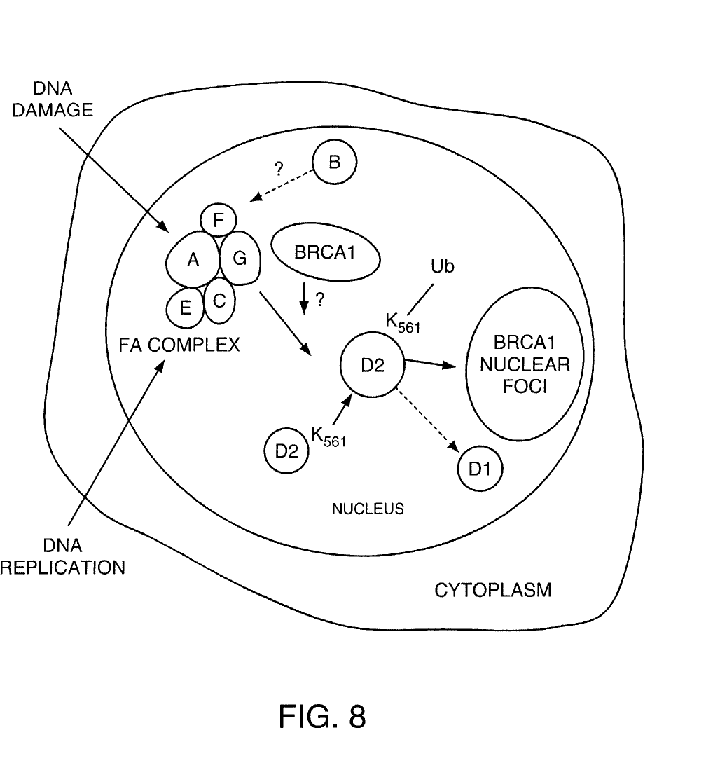

These features are reflective of the involvement of FA in maintenance of hematopoietic and gonadal stem cells, as well as the normal embryonic development of many different structures, including the skeleton and urogenital systems. Cell samples from patients were analyzed to determine defects in the FA complementation group D. Lymphoblasts from one patient gave rise to the PD20 cell line which was found to be mutated in a different gene from HSC 62 derived from another patient with a defect in the D complementation group Mutations from both patients mapped to the D complementation group but to different genes hence the naming of two FANCD proteins-FANCD1 (HSC 62) and FANCD2 (PD20) (Timmers et al., (2001) Molecular Cell, Vol. 7, pp. 241-248). We have shown that FANCD2 is the endpoint of the FA pathway and is not part of the FA nuclear complex nor required for its assembly or stability and that FANCD2 exists in two isoforms, FANCD2-S and FANCD2-L. We have also shown that transformation of the protein short form (FAND2-S) to the protein long form (FANCD2-L) occurs in response to the FA complex (FIG. 8). Defects in particular proteins associated with the FA pathway result in failure to make an important post translationally modified form of FANCD2 identified as FANCD2-L. The two isoforms of FANCD2 are identified as the short form and the long form.

Failure to make FANCD2-L correlates with errors in DNA repair and cell cycle abnormalities associated with diseases listed above.

To understand more about the role of FANCD2 in the aforementioned syndromes, we cloned the FANCD2 gene and determined the protein sequence. The FANCD2 gene has an open reading frame of 4,353 base pairs and forty four exons which encodes a novel 1451 amino acid nuclear protein, with a predicted molecular weight of 166 kD. Western blot analysis revealed the existence of 2 protein isoforms of 162 and 155 kD. The sequence corresponding to the 44 IntronlExon Junctions are provided in Table 6 (SEQ ID NO: 9-94).

Unlike previously cloned FA proteins, FANCD2 proteins from several non-vertebrate eurkaryotes showed highly significant alignment scores with proteins in D. melanogaster, A. thaliana, and C. elegans. The drosophila homologue, has 28% amino acid identity and 50% similarity to FANCD2 (FIG. and SEQ ID NO: 1-3) and no functional studies have been carried out in the respective species. No proteins similar to FANCD2 were found in E. coli or S. cerevisiae.

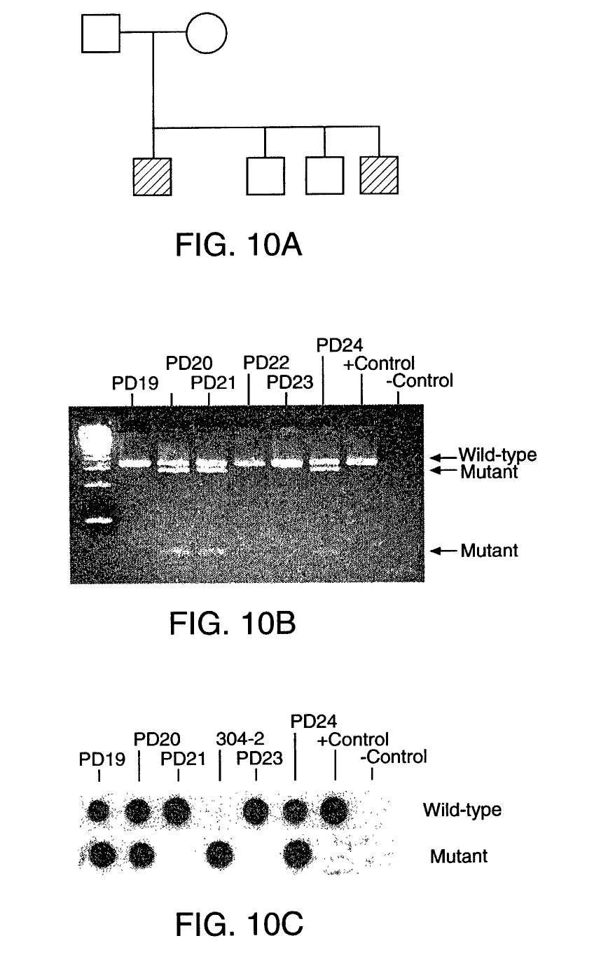

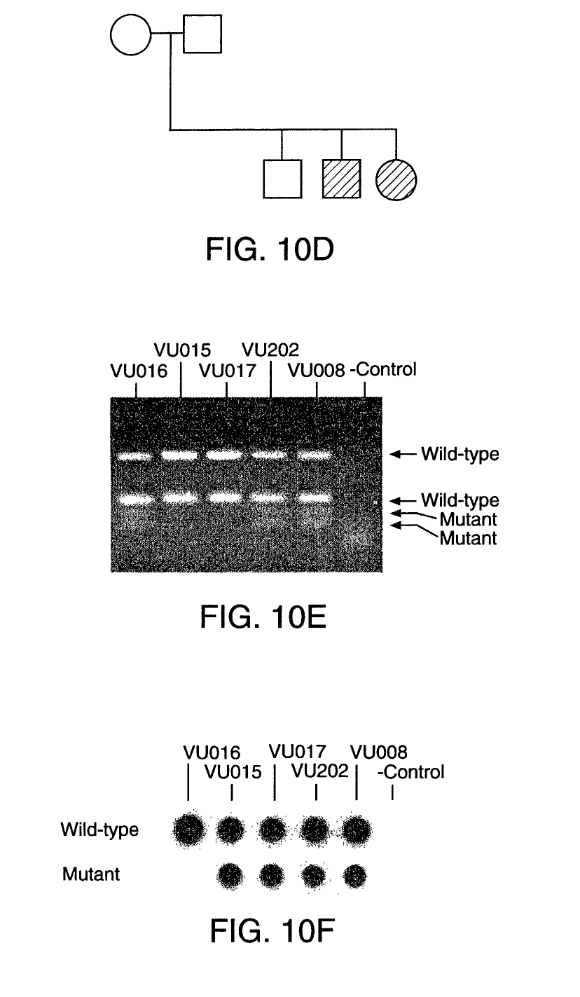

We obtained the FANCD2 DNA sequence (SEQ ID No: 5) by analyzing the chromosome 3p locus in PD20 and VU008, two FA cell lines having biallelic mutations in the FANCD2 gene (FIG. 10). The cell lines were assigned as complementation group D because lymphoblasts from the patients failed to complement HSC62, the reference cell line for group D. FANCD2 mutations were not detected in this group D reference cell line which indicates that the gene mutated in HSC62 is the gene encoding FANCD1 and in PD20 and VU008 is FANCD2 (FIG. 11). Microcell mediated chromosome transfer was used to identify the mutations (Whitney et al. Blood (1995) Vol. 88, 49-58). Detailed analysis of five microcell hybrids containing small overlapping deletions encompassing the locus narrowed the candidate region of the FANCD2 gene to 20OKb. The FANCD2 gene was isolated as follows: Three candidate ESTs were localized in or near this FANCD2 critical region. Using 5′ and 3′ RACE to obtain full-length cDNAs, the genes were sequenced, and the expression pattern of each was analyzed by northern blot. EST SGC34603 had ubiquitous and low level expression of a 5 kb and 7 kb MRNA similar to previously cloned FA genes. Open reading frames were found for TIGR-A004X28, AA609512 and SGC34603 and were 234, 531 and 4413 bp in length respectively. All 3 were analyzed for mutations in PD20 cells by sequencing cloned RT-PCR products. Whereas no sequence changes were detected in TIGR-A004X28 and AA609512, five sequence changes were found in SGC34603. Next, we determined the structure of the SGC34603 gene by using cDNA sequencing primers on BAC 177N7 from the critical region.

Based on the genomic sequence information, PCR primer pairs were designed (Table 7), the exons containing putative mutations were amplified, and allele-specific assays were developed to screen the PD20 family as well as 568 control chromosomes. Three of the alleles were common polymorphisms; however, 2 changes were not found in the controls and thus represented potential mutations (Table 3). The first was a maternally inherited A−>G change at nt 376. In addition to changing an amino acid (S126G), this alteration was associated with mis-splicing and insertion of 13 bp from intron 5 into the mRNA. 43/43 (100%) independently cloned RT-PCR products with the maternal mutation contained this insertion, whereas only 3 % (1/31) of control cDNA clones displayed mis-spliced mRNA. The 13 bp insertion generated a frame-shift and predicts a severely truncated protein only 180 amino acids in length. The second alteration was a paternally inherited missense change at position 1236 (R1236H). The segregation of the mutations in the PD20 core family is depicted in FIG. 10. Because the SGC34603 gene of PD20 contained both a maternal and a paternal allele not present on 568 control chromosomes and because the maternal mutation was associated with mis-splicing in 100% of cDNAs analyzed, we concluded that SGC34603 is the FANCD2 gene.

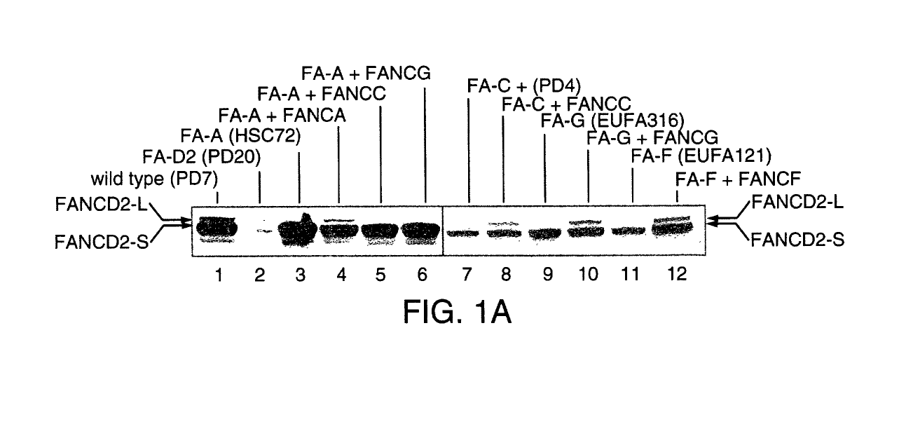

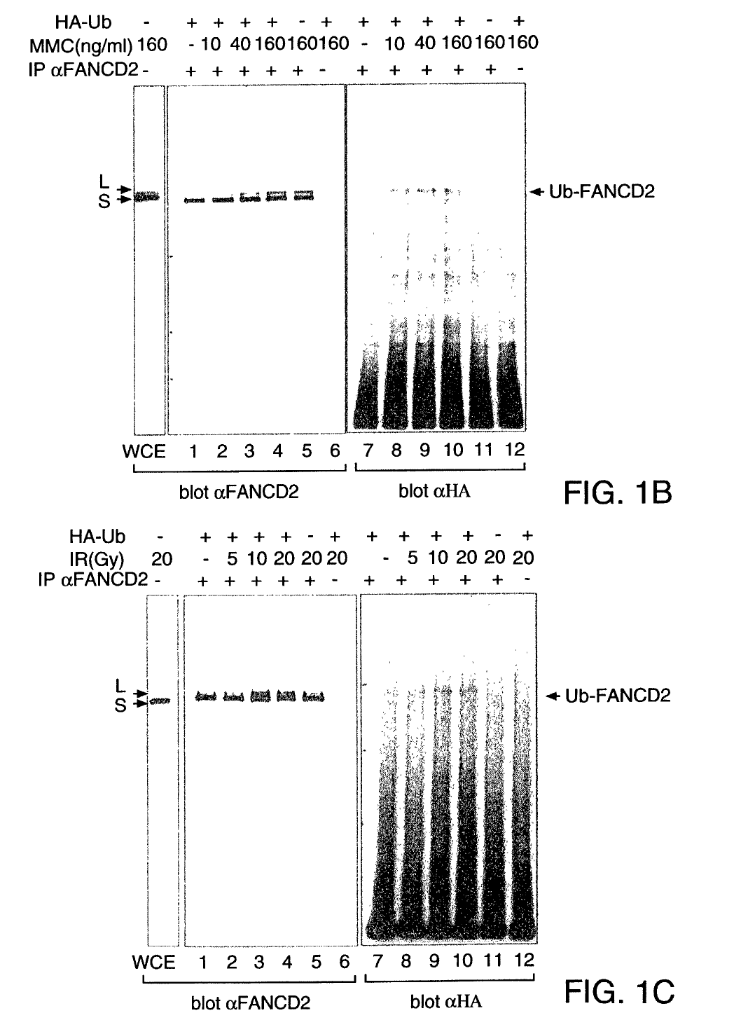

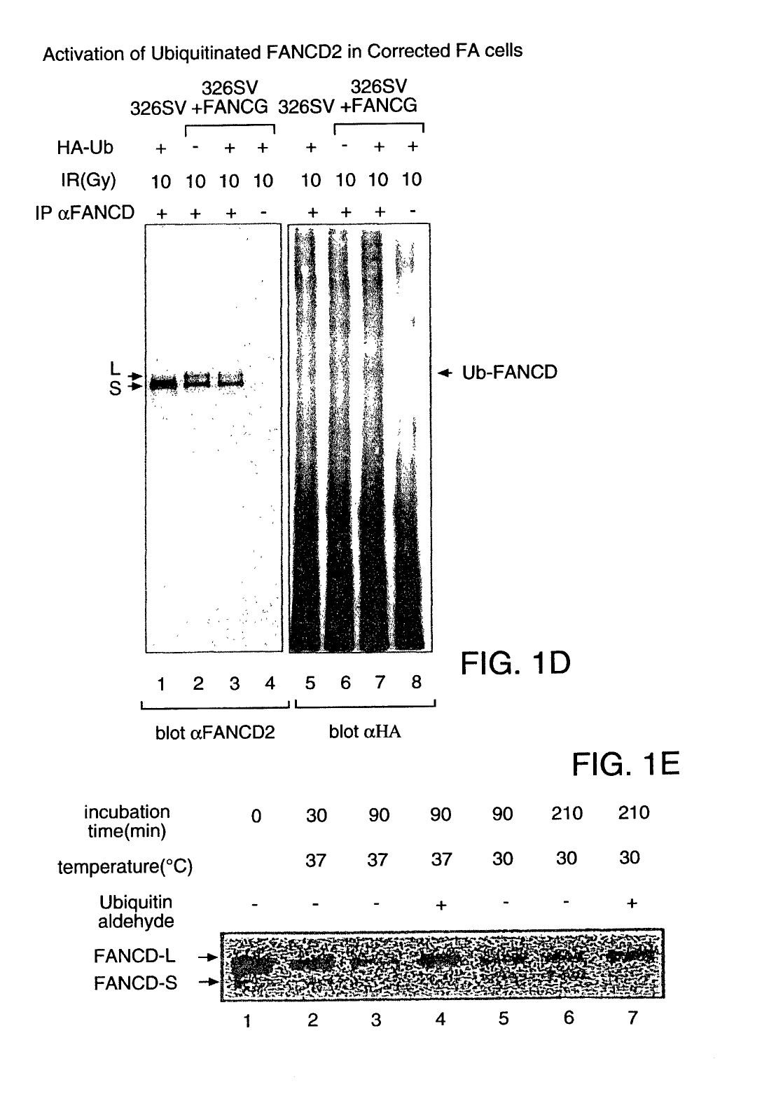

The protein encoded by FANCD2 is absent in PD20: To further confirm the identity of SGC34603 as FANCD2, an antibody was raised against the protein, and Western blot analysis was performed (FIG. 11). The specificity of the antibody was shown by retroviral transduction and stable expression FANCD2 in PD20 cells (FIG. 11c). In wild-type cells this antibody detected two bands (155 and 162 kD) which we call FANCD2-S and -L (best seen in FIG. 11c). FANCD2 protein levels were markedly diminished in all MMC-sensitive cell lines from patient PD20 (FIG. 1 la, lanes 2,4) but present in all wild-type cell lines and FA cells from other complementation groups. Furthermore, PD20 cells corrected by microcell-mediated transfer of chromosome 3 also made normal amounts of protein (FIG. 11a, lane 3).

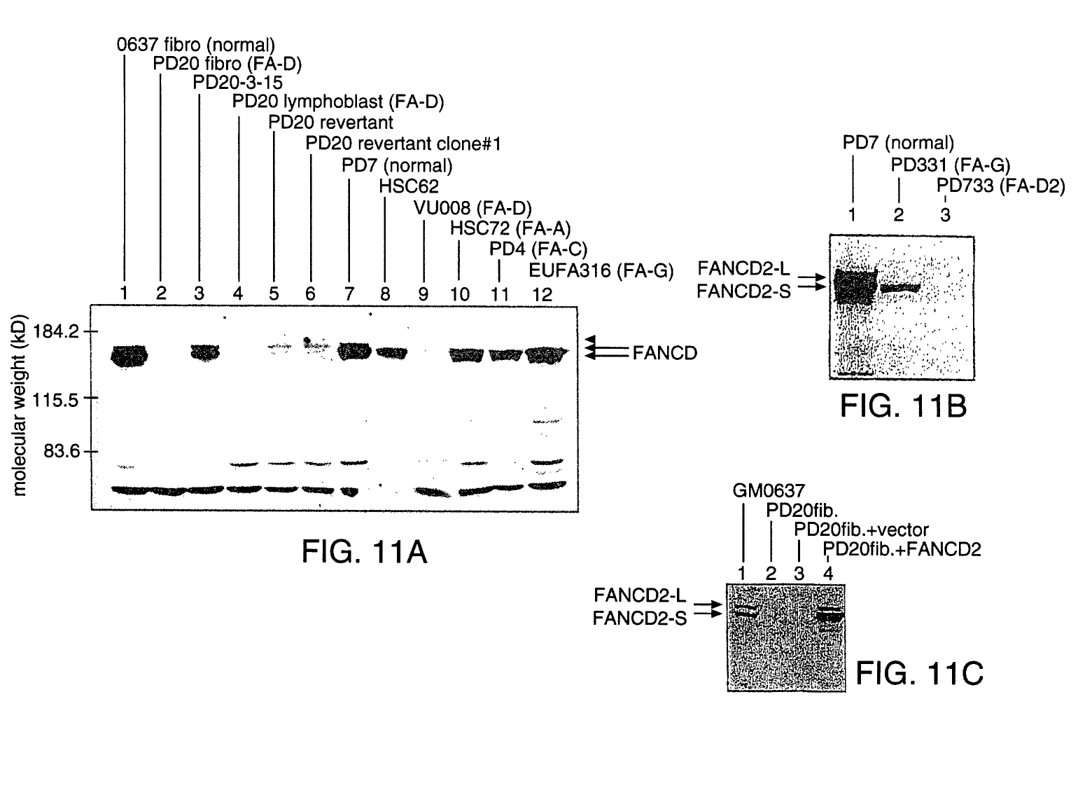

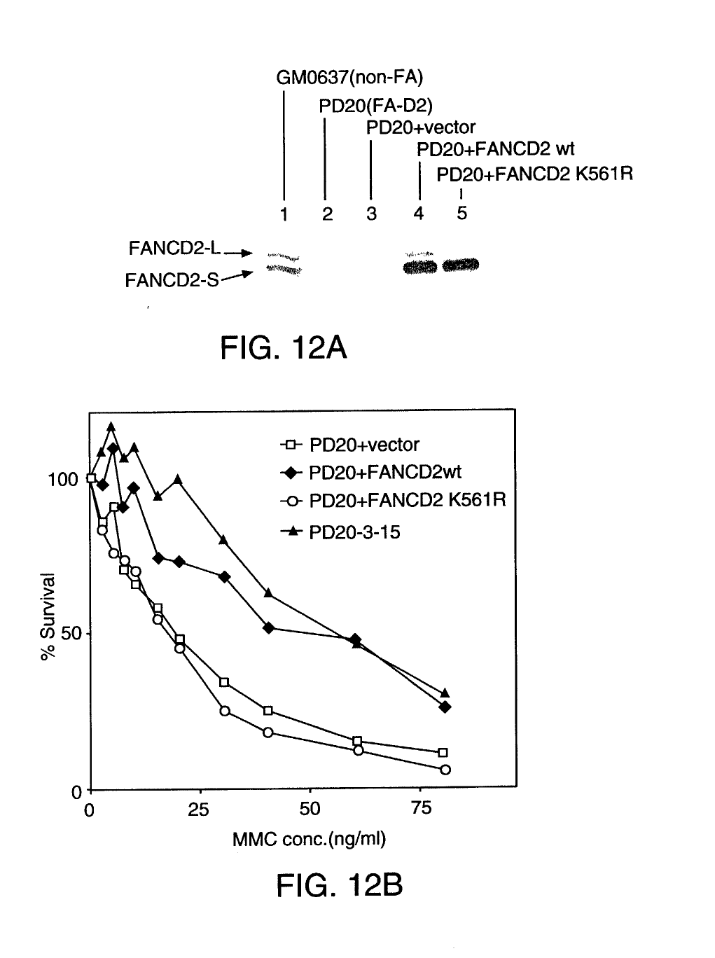

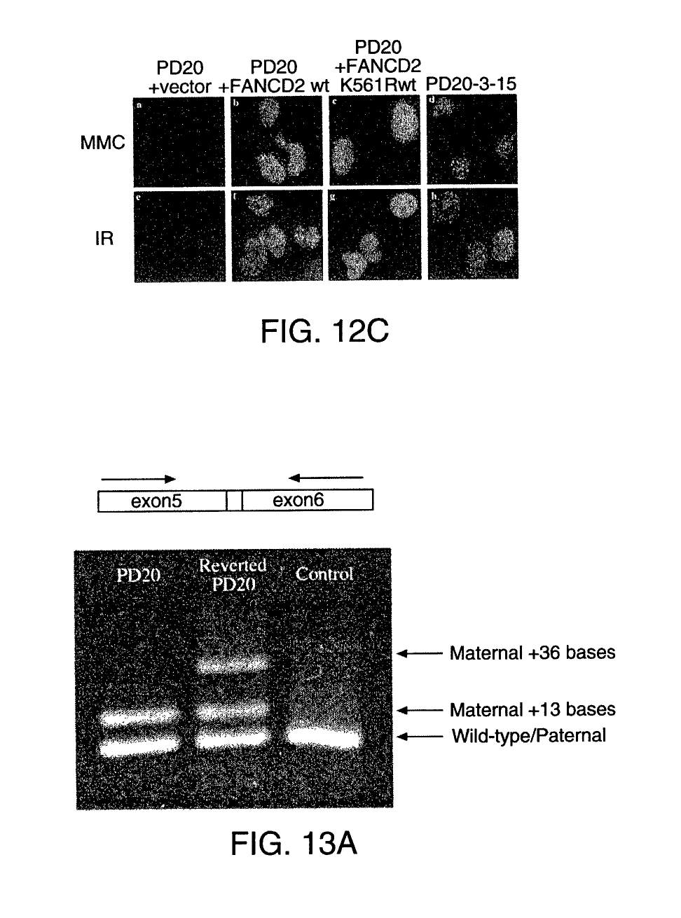

Functional complementation of FA—D2 cells with the FANCD2 cDNA: We next assessed the ability of the cloned FANCD2 cDNA to complement the MMC sensitivity of FA—D2 cells (FIG. 12). The full length FANCD2 cDNA was subcloned into the retroviral expression vector, pMMP-puro, as previously described (Pulsipher, et al., (1998) Mol. Med., Vol. 4, pp. 468-479). The transduced PD-20 cells expressed both isoforms of the FANCD2 protein, FANCD2-S and FANCD2-L (FIG. 12c). Transduction of FA—D2 (PD20) cells with pMMP-FANCD2 corrected the MMC sensitivity of the cells. These results further show that the cloned FANCD2 cDNA encodes the FANCD2-S protein, which can be post-translationally-modified to the FANCD2-L isoform. This important modification is discussed in greater detail below.

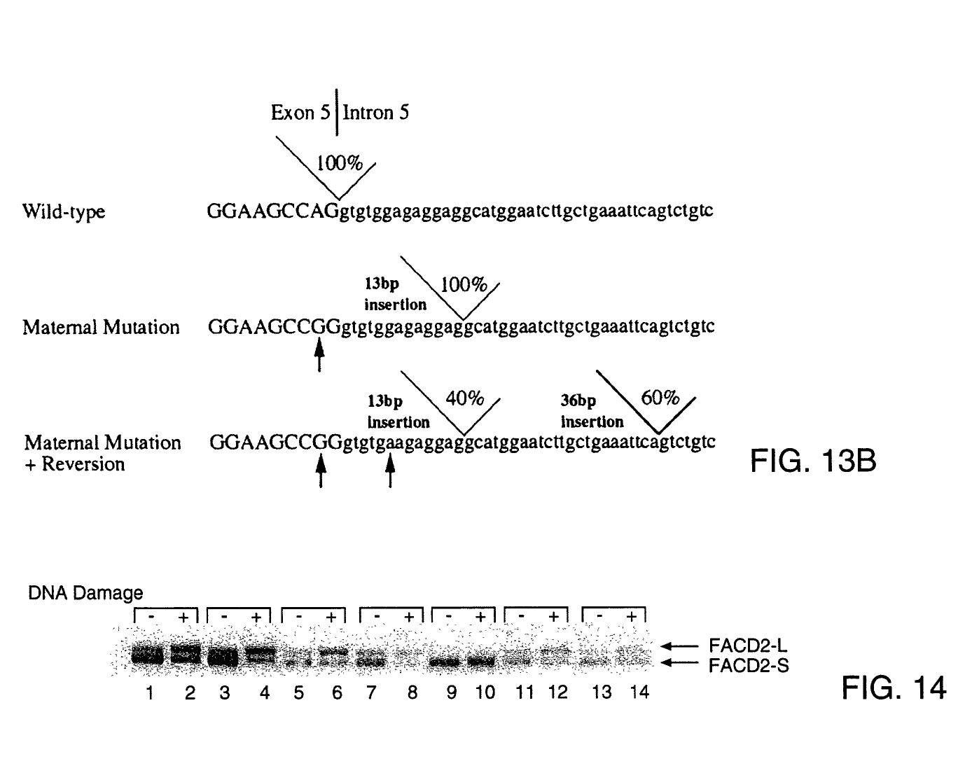

Analysis of a phenotypically reverted PD20 clone: We next generated additional evidence demonstrating that the sequence variations in PD20 cells were not functionally neutral polymorphisms. Towards this end we performed a molecular analysis of a revertant lymphoblast clone (PD20-cl.1) from patient PD20 which was no longer sensitive to MMC. Phenotypic reversion and somatic mosaicism are frequent findings in FA and have been associated with intragenic events such as mitotic recombination or compensatory frame-shifts. Indeed, ˜60% of maternally derived SGC34603 cDNAs had a novel splice variant inserting 36 bp of intron 5 sequence rather than the usually observed 13 bp (FIG. 13). The appearance of this in-frame splice variant correlated with a de novo base change at position IVS5+6 from G to A (FIG. 13) and restoration of the correct reading frame was confirmed by Western blot analysis. In contrast to all MMC sensitive fibroblasts and lymphoblasts from patient PD20, PD20-cl. 1 produced readily detectable amounts of FANCD2 protein of slightly higher molecular weight than the normal protein.

Analysis of cell lines from other “FANCD” patients: The antibody was also used to screen additional FA patient cell lines, including the reference cell line for FA group D, HSC and 2 other cell lines identified as group D by the European Fanconi Anemia Registry (EUFAR). VU008 did not express the FANCD2 protein and was found to be a compound heterozygote, with a missense and nonsense mutation, both in exon 12, and not found on 370 control chromosomes (Table 3, FIG. 11). The missense mutation appears to destabilize the FANCD2 protein, as there is no detectable FANCD2 protein in lysates from VU008 cells. A third patient PD733 also lacked FANCD2 protein (FIG. 11b, lane 3) and a splice mutation leading to absence of exon 17 and an internal deletion of the protein was found. The correlation of the mutations with the absence of FANCD2 protein in cell lysates derived from these patients substantiates the identity of FANCD2 as a FA gene. In contrast, readily detectable amounts of both isoforms of the FANCD2 protein were found in HSC 62 (FIG. 11a, lane 8) and VU423 cDNA and genomic DNA from both cell lines were extensively analyzed for mutations, and none were found. In addition, a whole cell fusion between VU423 and PD 20 fibroblasts showed complementation of the chromosome breakage phenotype (Table 5). Taken together these data show that FA group D are genetically heterogeneous and that the gene(s) defective in HSC 62 and VU423 are distinct from FANCD2.

The identification and sequencing of the FANCD2 gene and protein provides a novel target for therapeutic development, diagnostic tests and screening assays for diseases associated with failure of DNA repair and cell cycle abnormalities including but not limited to those listed in Table 2.

The following description provides novel and useful insights into the biological role of FANCD2 in the FA pathway which provides a basis for diagnosis and treatment of the aforementioned syndromes.

Evidence that FA cells have an underlying molecular defect in cell cycle regulation include the following: (a) FA cells display a cell cycle delay with 4N DNA content which is enhanced by treatment with chemical crosslinking agents, (b) the cell cycle arrest and reduced proliferation of FA cells can be partially corrected by overexpression of a protein, SPHAR, a member of the cyclin family of proteins and (c) caffeine abrogates the G2 arrest of FA cells. Consistent with these results, caffeine constitutively activates cdc2 and may override a normal G2 cell cycle checkpoint in FA cells. Finally, the FANCC protein binds to the cyclin dependent kinase, cdc2. We propose that the FA complex may be a substrate or modulator of the cyclinB/cdc2 complex.

Additionally, evidence that FA cells have an underlying defect in DNA repair is suggested by (a) FA cells that are sensitive to DNA cross-linking agents and ionizing radiation (IR), suggesting a specific defect in the repair of cross-linked DNA or double strand breaks, (b) DNA damage of FA cells which results in a hyperactive p53 response, suggesting the presence of defective repair yet intact checkpoint activities; and (c) FA cells with a defect in the fidelity of non-homologous end joining and an increased rate of homologous recombination (Garcia-Higuera et al. Mol. Cell. (2001), Vol. 7, pp. 249-262), (Grompe et al., Hum. Mol. Genet. (2001) Vol. 10, pp. 1-7).

Despite these general abnormalities in cell cycle and DNA repair. The mechanism by which FA pathway regulates these activities has remained elusive. Here we show that the FANCD2 protein functions downstream of the FA protein complex. In the presence of the assembled FA protein complex, the FANCD2 protein is activated to a high molecular weight, monoubiquitinated isoform which appears to modulate an S phase specific DNA repair response. The activated FANCD2 protein accumulates in nuclear foci in response to DNA damaging agents and co-localizes and co-immunoprecipitates with a known DNA repair protein, BRCA1. These results resolve previous conflicting models of the FA pathway (DAndrea et al., 1997) and demonstrate that the FA proteins cooperate in a cellular response to DNA damage.

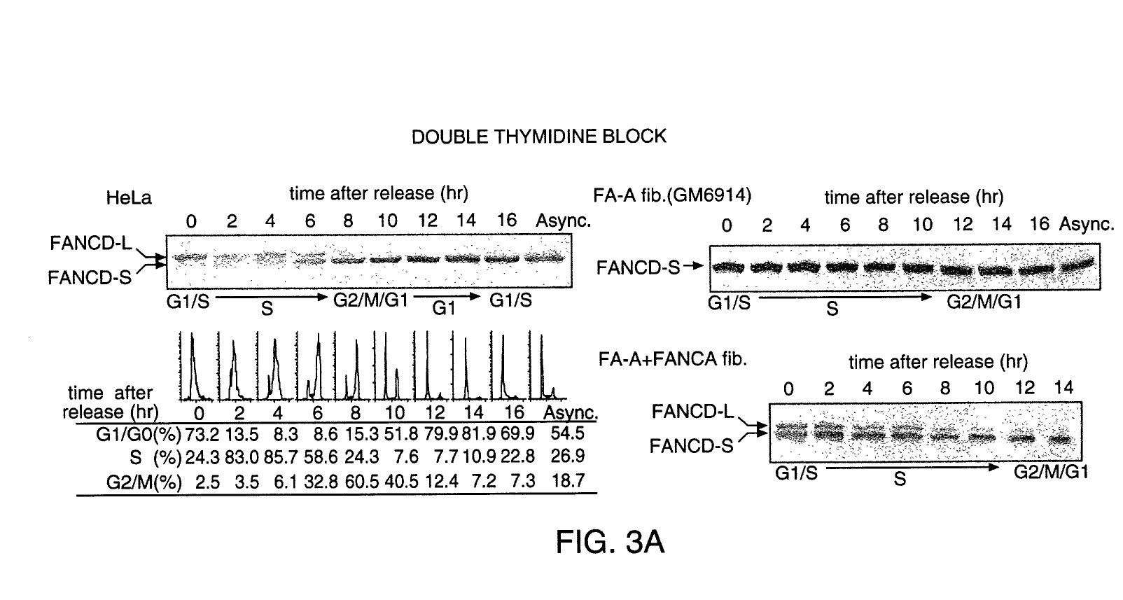

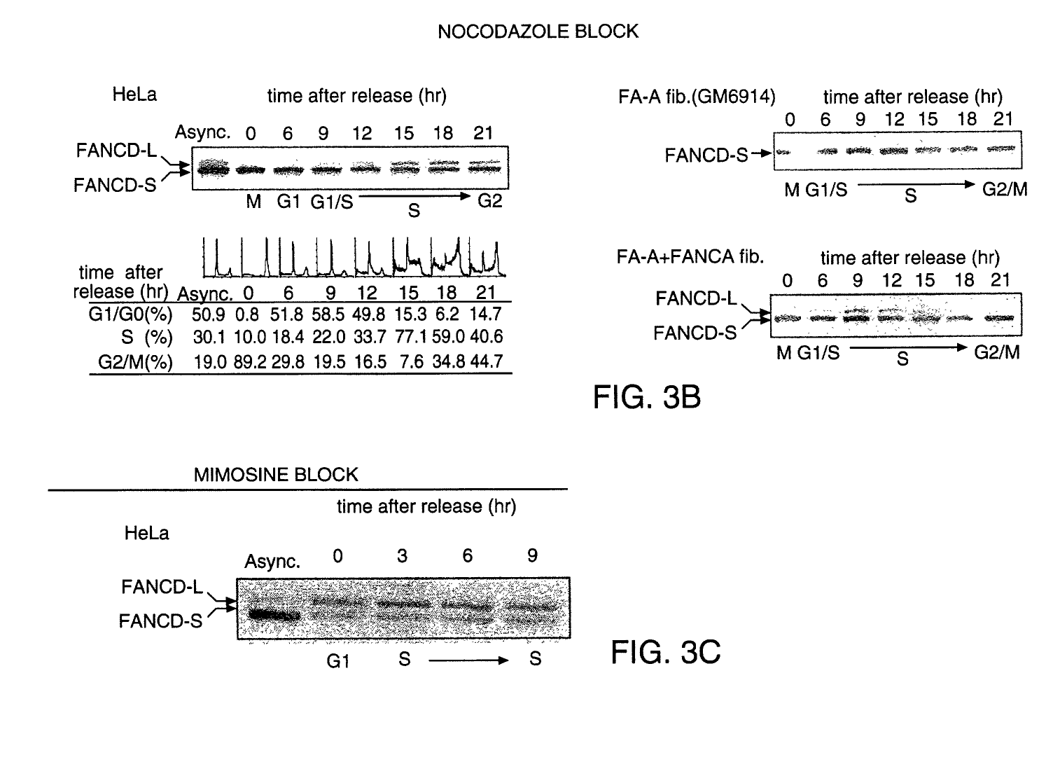

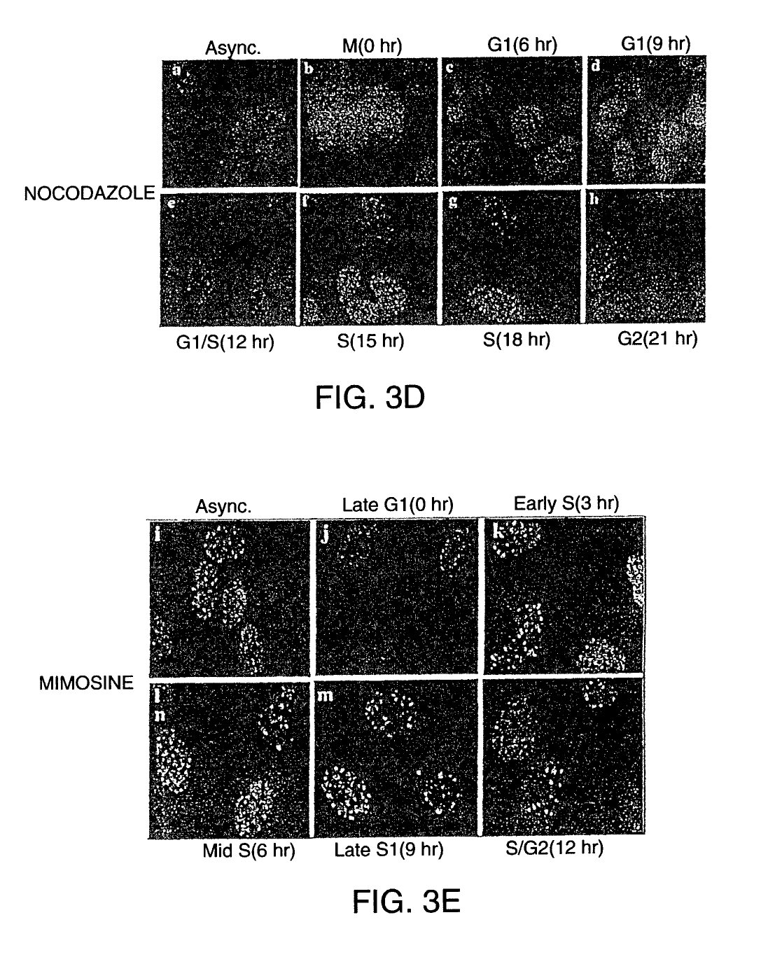

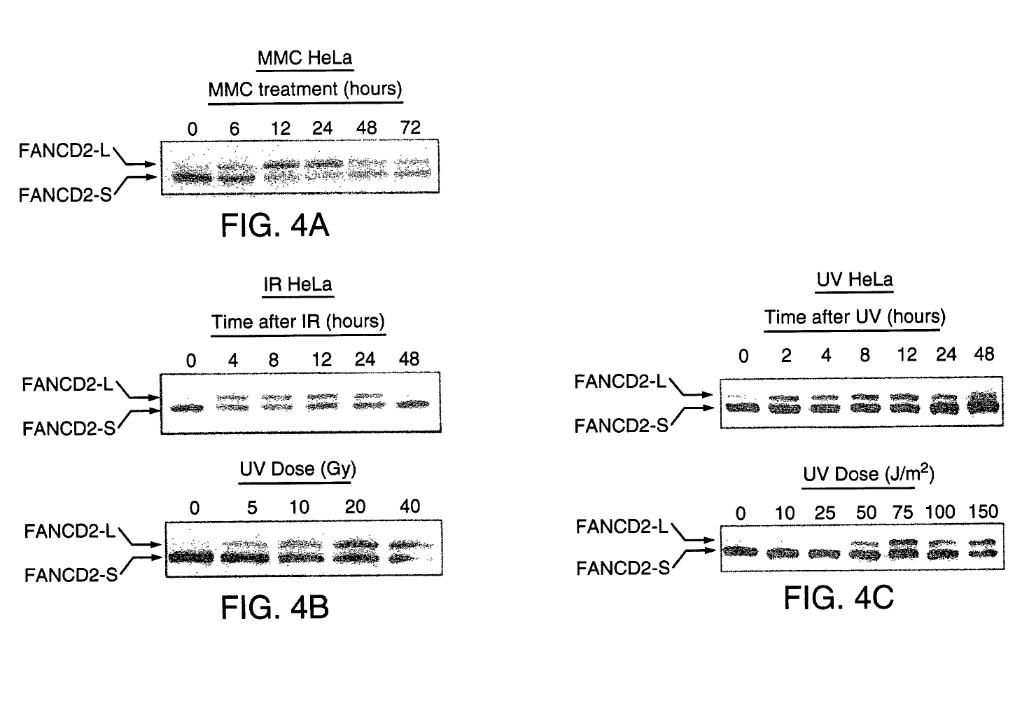

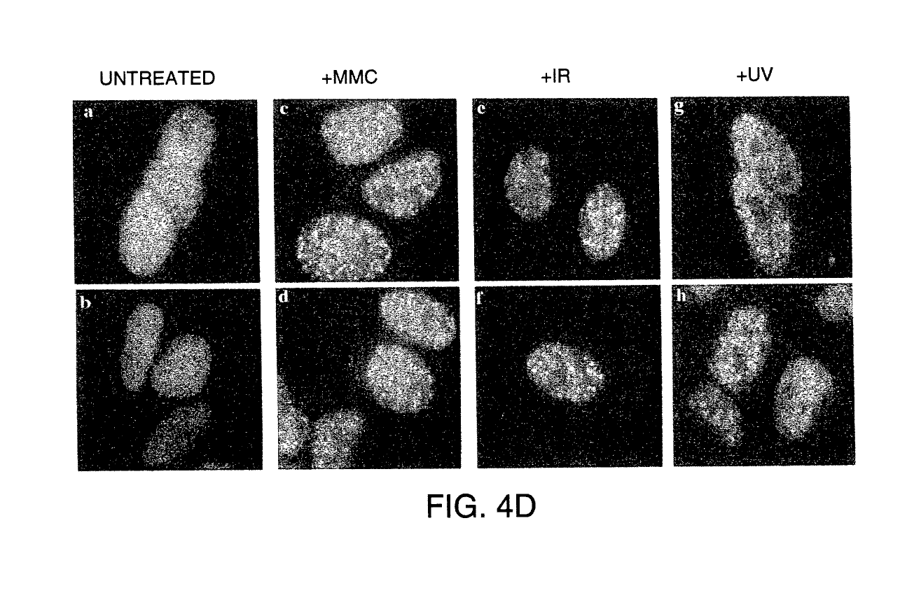

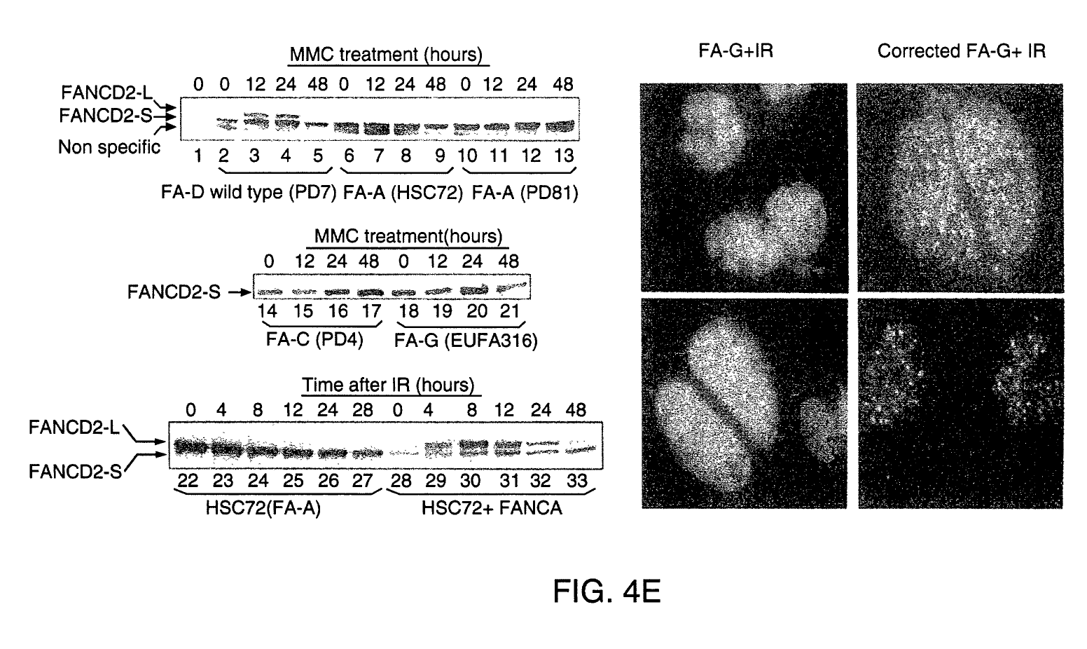

The FA pathway includes the formation of the FA multisubunit nuclear complex which in addition to AIC/G, we have shown also includes FANCF as a subunit of the complex (FIG. 8). The FA pathway becomes “active” during the S phase to provide S-phase specific repair response or checkpoint response. The normal activation of the FA pathway which relies on the FA multisubunit complex results in the regulated monoubiquitination of the phosphoprotein-FANCD2 via a phosphorylation step to a high molecular weight activated isoform identified as FANCD-2L (FIG. 1). Monoubiquitination is associated with cell trafficking. FANCD2-L appears to modulate an S-phase specific DNA repair response (FIG. 3). The failure of FA cells to activate the S phase-specific activation of FANCD2 is associated with cell cycle specific abnormalities. The activated FANCD2 protein accumulates in nuclear foci in response to the DNA damaging agents, MMC and IR, and colocalizes and co-immunoprecipitates with a known DNA repair protein, BRCA1 (FIG. 4-6). These results resolve previous conflicting models of FA protein function (D'Andrea et. al., 1997) and strongly support a role of the FA pathway in DNA repair.

We have identified for the first time, an association between FANCD2 isoforms with respect to the FA pathway and proteins that are known diagnostic molecules for various cancers. A similar pathway with respect to DNA damage for the BRCA 1 protein which is activated to a high molecular weight, post-translationally-modified isoform in S phase or in response to DNA damage suggests that activated FANCD2 protein interacts with BRCA1. More particularly, the regulated monoubiquitination of FANCD2 appears to target the FANCD2 protein to nuclear foci containing BRCA1. FANCD2 co-immunoprecipitates with BRCA1, and may further bind with other “dot” proteins, such as RAD50, Mrel 1, NBS, or RAD5 1. Recent studies demonstrate that BRCA1 foci are composed of a large (2 Megadalton) multi-protein complex (Wang et al., (2000) Genes Dev., Vol. 14, pp. 927-939). This complex includes ATM, ATM substrates involved in DNA repair functions (BRCAl), and ATM substrates involved in checkpoint functions (NBS). It is further suggested that damage recognition and activation of the FA pathway involve kinases which respond to DNA damage including ATM, ATR, CHK1, or CHK2.

We have found that the DNA damaging reagents, IR and MMC, activate independent post-translational modifications of FANCD2 result in distinct functional consequences. IR activates the ATM dependent phosphorylation of FANCD2 at Serine 222 resulting in an S phase checkpoint response. MMC activates the BRACA-1 dependent and FA pathway dependent monouniquitination of FANCD2 at lysine 561, resulting in the assembly of FANCD2/BRCAl nuclear foci and MMC resistance. FANCD2 therefore has two independent functional roles in the maintenance of chromosomal stability resulting from two discrete post-translational modifications provide a link between two additional cancer susceptibility genes (ATM and BRCA1) in a common pathway. Several additional lines of evidence support an interaction between FANCD2 and BRCA1. First, the BRCA1 (−/−) cell line, HCC1937 (Scully, et al., (1999) Mol. Cell, Vol. 4, pp. 1093-1099) has a “Fanconi Anemia-like” phenotype, with chromosome instability and increased tri-radial and tetra-radial chromosome formations.

Second, although FA cells form BRCA1 foci (and RAD51 foci) normally in response to IR, BRCA1 (−/−) cells have no detectable BRCAl foci and a greatly decreased number of FANCD2 foci compared to normal cells. Functional complementation of BRCA (−/−) cells restored BRCAl foci and FANCD2 foci to normal levels, and restored normal MMC resistance.

The amount of FANCD2-L is determined in part by the amount of FAND2-S that is synthesized from the fancd2 gene and in part by the availability of the FA complex to monoubiquinated FANCD2-S to form FANCD2-L. The association of FANCD2-L with nuclear foci including BRCA and ATM and determining the role of FANCD2-L in DNA repair make this protein a powerful target for looking at potential cancer development in patients for a wide range of cancers. Such cancers include those that arise through lesions on chromosome 3p as well as cancers on other chromosomes such that mutations result in interfering with production of upstream members of the FA pathway such as FANCG, FANCC or FANCA. Cancer lines and primary cells from cancer patients including tumor biopsies are being screened for FANCD-L and abnormal levels of this protein is expected to correlate with early diagnosis of disease. Because FANCD2 protein is a final step in a pathway to DNA repair, it is envisaged that any abnormality in a protein in the one or more pathways that lead to the conversion of FANCD2-S to FANCD-L will be readily detected by measuring levels of FANCD2. Moreover, levels of FANCD2 affect how other proteins such as BRCA and ATM functionally interact in the nucleus with consequences for the patient. Analysis of levels of FANCD2 in a patient is expected to aid a physician in a clinical decision with respect to understanding the class of cancer presented by the patient. For instance, if a cancer cell fails to generate the monoubiquinated FANCD2-L isoform, the cell may have increased chromosome instability and perhaps increased sensitivity to irradiation or chemotherapeutic agents. This information will assist the physician in procedure improved treatment for the patient.

Fanconi Anemia is associated not only with a broad spectrum of different cancers but also with congenital abnormalities. Development of the fetus is a complex but orderly process. Certain proteins have a particularly broad spectrum of effects because they disrupt this orderly progression of development. The FA pathway plays a significant role in development and disruption of the FA pathway results in a multitude of adverse effects. Errors in the FA pathway are detectable through the analysis of the FAND2-L protein from fetal cells. FANCD2 represents a diagnostic marker for normal fetal development and a possible target for therapeutic intervention.

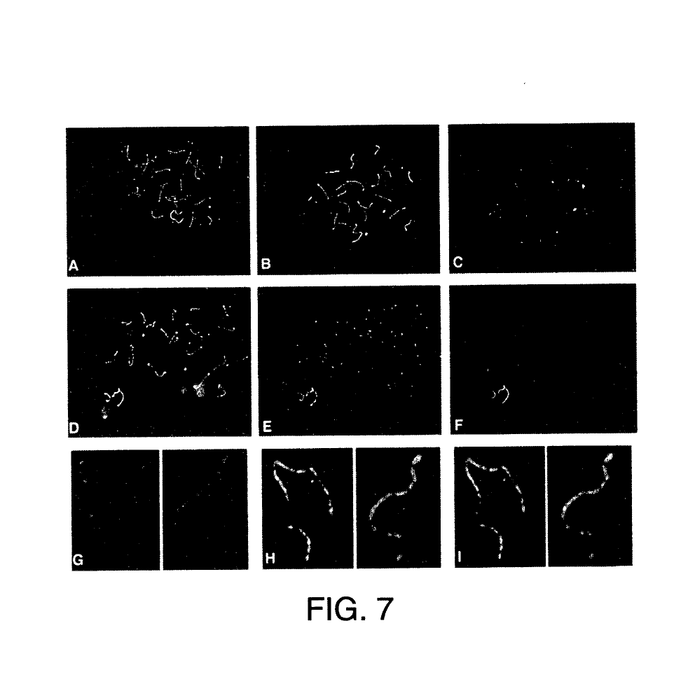

Consistent with the above, we have shown that FANCD2 plays a role in the production of viable sperm. FANCD2 fonns foci on the unpaired axes of chromosomes XY bivalents in late pachytene and in diplotene munine spermatocytes. (FIG. 7) Interestingly, FANCD2 foci are also seen at the autosomal telomeres in diplonema. Taken together with the known fertility defects in FA patients and FA—C knockout mice, our observations suggest that activated FANCD2 protein is required for normal progression of spermatocytes through meiosis I. Most of the FANCD2 foci seen on the XY axes were found to colocalize with BRCA1 foci, suggesting that the two proteins may function together in meiotic cells. Like BRCA1, FANCD2 was detected on the axial (unsynapsed) elements of developing synaptonemal complexes. Since recombination occurs in synapsed regions, FANCD2 may function prior to the initiation of recombination, perhaps to help prepare chromosomes for synapsis or to regulate subsequent recombinational events. The relatively synchronous manner in which FANCD2 assembles on meiotic chromosomes, and forms dot structures in mitotic cells, suggests a role of FANCD2 in both mitotic and meiotic cell cycle control.

Embodiments of the invention are directed to the use of the post translationally modified isoform: FANCD-2L as a diagnostic target for determining the integrity of the FA pathway. Ubiquitination of FANCD2 and the formation of FANCD2 nuclear foci are downstream events in the FA pathway, requiring the function of several FA genes. We have found that biallelic mutations of any of the upstream FA genes (FANCA, FANCB, FANCC, FANCE, FANCF and FANCG) block the posttranslational modification of FANCD2 the unubiquitinated FANCD2 (FANCD2-S) form to the ubiquitinated (FANCD2-L). Any of these upstream defects can be overridden by transfecting cells with FANCD2 cDNA. (FIG. 1a)

We have demonstrated for the first time the existence of FANCD2 and its role in the FA pathway. We have shown that FANCD2 accumulates in nuclear foci in response to DNA damaging agents where it is associated with other DNA repair proteins such as BRCA 1 and ATM. We have also demonstrated that FANCD2 exists in two isoforms in cells where a reduction in one of the two isoforms, FANCD2-L is correlated with Fanconi Anemia and with increased cancer susceptibility. We have used these findings to propose a number of diagnostic tests for use in the clinic that will assist with patient care.

These tests include: (a) genetic and prenatal counseling for parents concerned about inherited Fanconi Anemia in a future offspring or in an existing pregnancy; (b) genetic counseling and immunodiagnostic tests for adult humans to determine increased susceptibility to a cancer correlated with a defective FA pathway; and (c) diagnosing an already existing cancer in a subject to provide an opportunity for developing treatment protocols that are maximally effective for the subject while minimizing side effects.

The diagnostic tests described herein rely on standard protocols known in the art for which we have provided novel reagents to test for FANCD2 proteins and nucleotide sequences. These reagents include antibodies specific for FANCD2 isoforms, nucleotide sequences from which vectors, probes and primers have been derived for detecting genetic alterations in the FANCD2 gene and cells lines and recombinant cells for preserving and testing defects in the FA pathway.

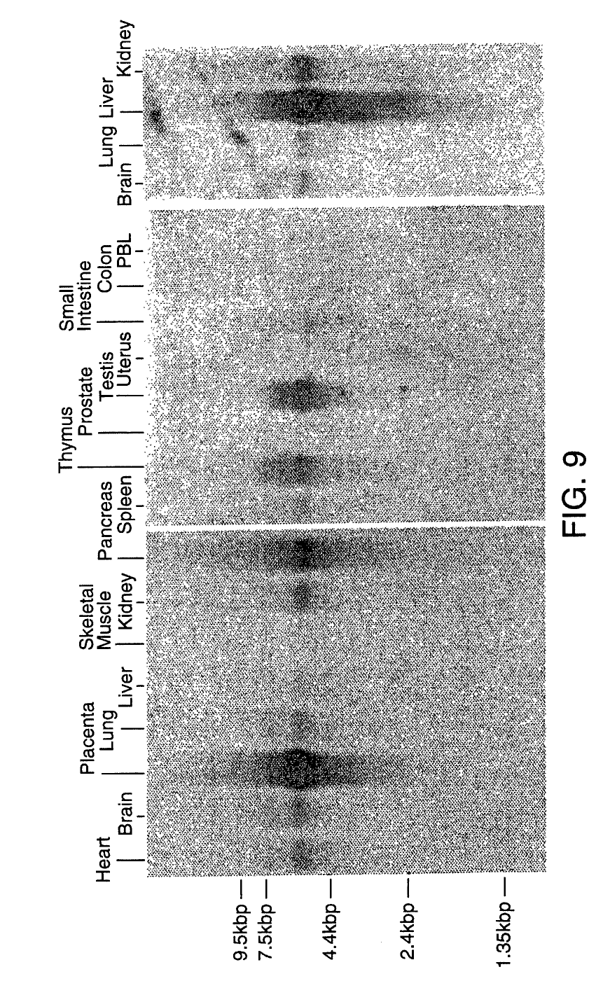

We have prepared monoclonal and polyclonal antibody preparations as described in Example 1 that are specific for FANCD2-L and FANCD2-S proteins. In addition, FANCD2 isoform specific antibody fragments and single chain antibodies may be prepared using standard techniques. We have used these antibodies in wet chemistry assays such as immunoprecipitation assays, for example Western blots, to identify FANCD2 isoforms in biological samples (FIG. 1). Conventional immunoassays including enzyme linked immunosorbent assays (ELISA), radioimmune assays (RIA), immunoradiometric assays (IRMA) and immunoenzymatic assays (IEMA) and further including sandwich assays may also be used. Other immunoassays may utilize a sample of whole cells or lyzed cells that are reacted with antibody in solution and optionally analyzed in a liquid state within a reservoir. Isoforms of FANCD2 can be identified in situ in intact cells including cell lines, tissue biopsies and blood by immunological techniques using for example fluorescent activated cell sorting, and laser or light microscopy to detect immunofluorescent cells. (FIGS. 1-7, 9-14). For example, biopsies of tissues or cell monolayers, prepared on a slide in a preserved state such as embedded in paraffin or as frozen tissue sections can be exposed to antibody for detecting FANCD2-L and then examined by fluorescent microscopy.

In an embodiment of the invention, patient-derived cell lines or cancer cell lines are analyzed by immunoblotting and immunofluorescence to provide a novel simple diagnostic test for detecting altered amounts of FANCD2 isoforms. The diagnostic test also provides a means to screen for upstream defects in the FA pathway and a practical alternative to the currently employed DEB/MMC chromosome breakage test for FA because individuals with upstream defects in the FA pathway are unable to ubiquitinate FANCD2. Other assays may be used including assays that combine retroviral gene transfer to form transformed patient derived cell lines (Pulsipher et al. (1998). Mol Med 4, 468-79 together with FANCD2 immunoblotting to provide a rapid subtyping analysis of newly diagnosed patients with any of the syndromes described in Table 2, in particular, that of FA.

The above assays may be performed by diagnostic laboratories, or, alternatively, diagnostic kits may be manufactured and sold to health care providers or to private individuals for self-diagnosis. The results of these tests and interpretive information are useful for the healthcare provider in diagnosis and treatment of a patient's condition.

Genetic tests can provide for a subject, a rapid reliable risk analysis for a particular condition against an epidemiological baseline. Our data suggests that genetic heterogeneity occurs in patients with FA within the FANCD2 complementation group. We have found a correlation between genetic heterogeneity and disease as well as genetic heterogeneity and abnormal post-translational modifications that result in the presence or absence of FANCD2-L. This correlation provides the basis for prognostic tests as well as diagnostic tests and treatments for any of the syndromes characterized by abnormal DNA repair. For example, nucleic acid from a cell sample obtained from drawn blood or from other cells derived from a subject can be analyzed for mutations in the FANCD2 gene and the subject may be diagnosed to have an increased susceptibility to cancer

We have located the FANCD2 gene at 3p25.3 on chromosome 3p in a region which correlates to a high frequency of cancer. Cytogenetic and loss of heterozygosity (LOH) studies have demonstrated that deletions of chromosome 3p occur at a high frequency in all forms of lung cancer (Todd et al. Cancer Res. Vol. 57, pp. 1344-52). For example, homozygous deletions were found in three squamous cell lines within a region of 3p21. Homozygous deletions were also found in a small cell tumor at 3p12 and a 3p14.2. (Franklin et al. (1997) Cancer Res. 57:1344-52). The present mapping of FANCD2 is supportive of the theory that this chromosomal region contains important tumor suppressor genes. Further support for this has been provided by a recent publication of Sekine et al., Human Molecular Genetics, (2001) 10, pp. 1421-1429 who reported localization of a novel susceptibility gene for familial ovarian cancer to chromosome 3p22-p25. The reduction or absence of FANCD2-L is here proposed to be diagnostic for increased risk of tumors resulting from mutations not only at the FANCD2 site (3p25.3) but also at other sites in the chromosomes possibly arising from defects in DNA repair following cell damage arising from exposure to environmental agents and normal aging processes.

As more individuals and families are screened for genetic defects in the FANCD2 gene, a data base will be developed in which population frequencies for different mutations will be gathered and correlations made between these mutations and health profile for the individuals so that the predictive value of genetic analysis will continually improve. An example of an allele specific pedigree analysis for FANCD2 is provided in FIG. 10 for two families.

Diagnosis of a mutation in the FANCD2 gene may initially be detected by a rapid immunological assay for detecting reduced amounts of FANCD2-L proteins. Positive samples may then be screened with available probes and primers for defects in any of the genes in the FA pathway. Where a defect in the FANCD2 gene is implicated, primers or probes such as provided in Table 7 may be used to detect a mutation. In those samples, where a mutation is not detected by such primers or probes, the entire FANCD2 gene may be sequenced to determine the presence and location of the mutation in the gene.

Nucleic acid screening assays for use in identifying a genetic defect in the FANCD2 gene locus may include PCR and non PCR based assays to detect mutations. There are many approaches to analyzing cell genomes for the presence of mutations in a particular allele. Alteration of a wild-type FANCD2 allele, whether, for example, by point mutation, deletion or insertions can be detected using standard methods employing probes. (US Patent 6,033,857). Standard methods include: (a) fluorescent in situ hybridization (FISH) which may be used on whole intact cells.; and (b) allele specific oligonucleotides (ASO) may be used to detect mutations using hybridization techniques on isolated nucleic acid (Conner et al., 1989, Hum.Genet. 85: 55-74). Other techniques include (a) observing shifts in electrophoretic mobility of single-stranded DNA on non-denaturing polyacrylamide gels, (b) hybridizing a FANCD2 gene probe to genomic DNA isolated from the tissue sample, (c) hybridizing an allele-specific probe to genomic DNA of the tissue sample, (d) amplifying all or part of the FANCD2 gene from the tissue sample to produce an amplified sequence and sequencing the amplified sequence, (e) amplifying all or part of the FANCD2 gene from the tissue sample using primers for a specific FANCD2 mutant allele, (f) molecular cloning all or part of the FANCD2 gene from the tissue sample to produce a cloned sequence and sequencing the cloned sequence, (g) identifying a mismatch between (i) a FANCD2 gene or a FANCD2 mRNA isolated from the tissue sample, and (ii) a nucleic acid probe complementary to the human wild-type FANCD2 gene sequence, when molecules (i) and (ii) are hybridized to each other to form a duplex, (h) amplification of FANCD2 gene sequences in the tissue sample and hybridization of the amplified sequences to nucleic acid probes which comprise wild-type FANCD2 gene sequences, (I) amplification of FANCD2 gene sequences in the tissue sample and hybridization of the amplified sequences to nucleic acid probes which comprise mutant FANCD2 gene sequences, 0) screening for a deletion mutation in the tissue sample, (k) screening for a point mutation in the tissue sample, (1) screening for an insertion mutation in the tissue sample, and (m) in situ hybridization of the FANCD2 gene of the tissue sample with nucleic acid probes which comprise the FANCD2 gene.

It is often desirable to scan a relatively short region of a gene or genome for point mutations: The large numbers of oligonucleotides needed to examine all potential sites in the sequence can be made by efficient combinatorial methods (Southern, E. M et al., (1994). Nucleic Acids Res. 22:,1368-1373). Arrays may be used in conjunction with ligase or polymerase to look for mutations at all sites in the target sequence.(U.S. Pat. No. 6,307,039) Analysis of mutations by hybridization can be performed for example by means of gels, arrays or dot blots.

The entire gene may be sequenced to identify mutations. (US 6,033,857). Sequencing of the FANCD2 locus can be achieved using oligonucleotide tags from a minimally cross hybridizing set which become attached to their complements on solid phase supports when attached to target sequence (US 6,280,935).

Other approaches to detecting mutations in the FANCD2 gene include those described in U.S. Pat. Nos. 6,297,010, 6,287,772 and 6,300,076). It is further contemplated that the assays may employ nucleic acid microchip technology or analysis of multiple samples using laboratories on chips.

A subject who has developed a tumor maybe screened using nucleic acid diagnostic tests or antibody based tests to detect a FANCD2 gene mutation or a deficiency in FANCD2-L protein. On the basis of such screening samples may be obtained from subjects having a wide range of cancers including melanoma, leukemia, astocytoma, glioblastoma, lymphoma, glioma, Hodgkins lymphoma, chronic lymphocyte leukemia and cancer of the pancreas, breast, thyroid, ovary, uterus, testis, pituitary, kidney, stomach, esophagus and rectum. The clinician has an improved ability to select a suitable treatment protocol for maximizing the treatment benefit for the patient. In particular, the presence of a genetic lesion or a deficiency in FANCD2-L protein may be correlated with responsiveness to various existing chemotherapeutic drugs and radiation therapies.

New therapeutic treatments may be developed by screening for molecules that modulate the monoubiquitination of FANCD2-S to give rise to FANCD2-L in cell assays (Examples 11-12) and in knock-out mouse models (Example 10). Such molecules may include those that bind directly to FANCD2 or to molecules such as BRACA-2 that appears to interact with BRACA-1 which in turn appears to be activated by FANCD2.

In addition, to screening assays that rely on defects in the FANCD2 gene or protein, an observed failure of the ubiquitination reaction that is necessary for the formation of FANCD2-L may result from a defect in the FA pathway at any point preceding the post translational modification of FANCD2 including FANCD2-S itself. Knowing the terminal step in the reactions, enables a screening assay to be formulated in which small molecules are screened in cells containing “broken FA pathway” or in vitro until a molecule is found to repair the broken pathway. This molecule can then be utilized as a probe to identify the nature of the defect. It may further be used as a therapeutic agent to repair the defect. For example, we have shown that cell cycle arrest and reduced proliferation of FA cells can be partially corrected by overexpression of a protein, SPHAR, a member of the cyclin family of proteins This can form the basis of an assay which is suitable as a screen for identifying therapeutic small molecules.

Cells which are deficient in the posttranslational modified FANCD2 are particularly sensitive to DNA damage. These cells may serve as a sensitive screen for determining whether a compound (including toxic molecules) has the capability for damaging DNA. Conversely, these cells also serve as a sensitive screen for determining whether a compound can protect cells against DNA damage.

FA patients and patients suffering from syndromes associated with DNA repair defects die from complications of bone marrow failure. Gene transfer is a therapeutic option to correct the defect. Multiple defects may occur throughout the FA pathway. We have shown that the terminal step is critical to proper functioning of the cell and the organism. In an embodiment of the invention, correction of defects anywhere in the FA pathway may be satisfactorily achieved by gene therapy or by therapeutic agents that target the transformation of FANCD2-S to FANCD2-L so that this transformation is successfully achieved.

Gene therapy may be carried out according to generally accepted methods, for example, as described by Friedman in “Therapy for Genetic Disease,” T. Friedman, ed., Oxford University Press (1991), pp. 105-121. Targeted tissues for ex vivo or in vivo gene therapy include bone marrow for example, hematopoietic stem cells prior to onset of anemia and fetal tissues involved in developmental abnormalities. Gene therapy can provide wild-type FAND2-L function to cells which carry mutant FANCD2 alleles. Supplying such a function should suppress neoplastic growth of the recipient cells-or ameliorate the symptoms of Fanconi Anemia.

The wild-type FANCD-2 gene or a part of the gene may be introduced into the cell in a vector such that the gene remains extrachromosomal. In such a situation, the gene may be expressed by the cell from the extrachromosomal location. If a gene portion is introduced and expressed in a cell carrying a mutant FANCD-2 allele, the gene portion may encode a part of the FANCD-2 protein which is required for non-neoplastic growth of the cell. Alternatively, the wild-type FANCD-2 gene or a part thereof may be introduced into the mutant cell in such a way that it recombines with the endogenous mutant FANCD-2 gene present in the cell.

Viral vectors are one class of vectors for achieving gene therapy. Viral-mediated gene transfer can be combined with direct in vivo gene transfer using liposome delivery, allowing one to direct the viral vectors to the tumor cells and not into the surrounding nondividing cells. Alternatively, a viral vector producer cell line can be injected into tumors (Culver et al., 1992). Injection of producer cells would then provide a continuous source of vector particles. This technique has been approved for use in humans with inoperable brain tumors.

The vector may be injected into the patient, either locally at the site of the tumor or systemically (in order to reach any tumor cells that may have metastasized to other sites). If the transfected gene is not permanently incorporated into the genome of each of the targeted tumor cells, the treatment may have to be repeated periodically.

Vectors for introduction of genes both for recombination and for extrachromosomal maintenance are known in the art, (for example as disclosed in U.S. Pat. No. 5,252,479 and PCT 93/07282, and U.S. Pat. No. 6,303,379) and include viral vectors such as retroviruses, herpes viruses (US 6,287,557) or adenoviruses (US 6,281,010) or a plasmid vector containing the FANCD2-L.

A vector carrying the therapeutic gene sequence or the DNA encoding the gene or piece of the gene may be injected into the patient either locally at the site of a tumor or systemically so as to reach metastasized tumor cells. Targeting may be achieved without further manipulation of the vector or the vector may be coupled to a molecule having a specificity of binding for a tumor where such molecule may be a receptor agonist or antagonist and may further include a peptide, lipid (including liposomes) or saccharide including an oligopolysaccharide or polysaccharide) as well as synthetic targeting molecules. The DNA may be conjugated via polylysine to a binding ligand. If the transfected gene is not permanently incorporated into the genome of each of the targeted tumor cells, the treatment may have to be repeated periodically.

Methods for introducing DNA into cells prior to introduction into the patient may be accomplished using techniques such as electroporation, calcium phosphate co-precipitation and viral transduction as described in the art (U.S. Pat. No. 6,033,857), and the choice of method is within the competence of the routine experimenter

Cells transformed with the wild-type FANCD 2 gene or mutant FANCD 2 gene can be used as model systems to study remission of diseases resulting from defective DNA repair and drug treatments which promote such remission.

As generally discussed above, the FANCD 2 gene or fragment, where applicable, may be employed in gene therapy methods in order to increase the amount of the expression products of such genes in abnormal cells. Such gene therapy is particularly appropriate for use in pre-cancerous cells, where the level of FANCD 2-L polypeptide may be absent or diminished compared to normal cells and where enhancing the levels of FANCD2-L may slow the accumulation of defects arising from defective DNA repair and hence postpone initiation of a cancer state. It may also be useful to increase the level of expression of the FANCD 2 gene even in those cells in which the mutant gene is expressed at a “normal” level, but there is a reduced level of the FANCD 2-L isoform. The critical role of FANCD2-L in normal DNA repair provides an opportunity for developing therapeutic agents to correct a defect that causes a reduction in levels of FANCD2-L. One approach to developing novel therapeutic agents is through rational drug design. Rational drug design can provide structural analogs of biologically active polypeptides of interest or of small molecules with which they interact (e.g., agonists, antagonists, inhibitors or enhancers) in order to fashion more active or stable forms of the polypeptide, or to design small molecules which enhance or interfere with the function of a polypeptide in vivo. Hodgson, 1991. Rational drug design may provide small molecules or modified polypeptides which have improved FANCD2-L activity or stability or which act as enhancers, inhibitors, agonists or antagonists of FANCD2-L activity. By virtue of the availability of cloned FANCD2 sequences, sufficient amounts of the FANCD2-L polypeptide may be made available to perform such analytical studies as x-ray crystallography. In addition, the knowledge of the FANCD2-L protein sequence provided herein will guide those employing computer modeling techniques in place of, or in addition to x-ray crystallography.

Peptides or other molecules which have FANCD2-L activity can be supplied to cells which are deficient in the protein in a therapeutic formulation. The sequence of the FANCD2-L protein is disclosed for several organisms (human, fly and plant) (SEQ ID NO: 1-3). FANCD2 could be produced by expression of the cDNA sequence in bacteria, for example, using known expression vectors with additional posttranslational modifications. Alternatively, FANCD2-L polypeptide can be extracted from FANCD2-L-producing mammalian cells. In addition, the techniques of synthetic chemistry can be employed to synthesize FANCD2-L protein. Other molecules with FANCD2-L activity (for example, peptides, drugs or organic compounds) may also be used as a therapeutic agent. Modified polypeptides having substantially similar function are also used for peptide therapy.

Similarly, cells and animals which carry a mutant FANCD2 allele or make insufficient levels of FANCD2-L can be used as model systems to study and test for substances which have potential as therapeutic agents. The cells which may be either somatic or germline can be isolated from individuals with reduced levels of FANCD2-L. Alternatively, the cell line can be engineered to have a reduced levels of FANCD2-L, as described above. After a test substance is applied to the cells, the DNA repair impaired transformed phenotype of the cell is determined.

The efficacy of novel candidate therapeutic molecules can be tested in experimental animals for efficacy and lack of toxicity. Using standard techniques, animals can be selected after mutagenesis of whole animals or after genetic engineering of germline cells or zygotes to form transgenic animals. Such treatments include insertion of mutant FANCD2 alleles, usually from a second animal species, as well as insertion of disrupted homologous genes. Alternatively, the endogenous FANCD2 gene of the animals may be disrupted by insertion or deletion mutation or other genetic alterations using conventional techniques (Capecchi, 1989 Science Vol. 244, pp 1288-1292; Valancius and Smithies, 1991). After test substances have been administered to the animals, the growth of tumors must be assessed. If the test substance prevents or suppresses pathologies arising from defective DNA repair, then the test substance is a candidate therapeutic agent for the treatment of the diseases identified herein.

The present invention is described by reference to the following Examples, which are offered by way of illustration and are not intended to limit the invention in any manner. Standard techniques well known in the art or the techniques specifically described below were utilized.

All references cited herein are incorporated by reference.

### EXAMPLES

### Example 1

Experimental protocols used in Examples 2-8

Cell Lines and Culture Conditions. Epstein-Barr virus (EBV) transformed lymphoblasts were maintained in RPMI media supplemented with 15% heat-inactivated fetal calf serum (FCS) and grown in a humidified 5% C02-containing atmosphere at 37° C. A control lymphoblast line (PD7) and FA lymphoblast lines (FA—A (HSC72), FA—C (PD-4), FA—D (PD-20), FA—F (EUFA121), and FA—G (EUFA316)) have been previously described (de Winter et al., 1998, Nat.Genet. 20: 281-283) (Whitney et al., 1995, Nat.Genet. 11: 341-343) (Yamashita et al., 1994, P.N.A.S. 91: 6712-6716) (de Winter et al., 2000, Am.J.Hum.Genet. 67: 1306-1308). PD81 is a lymphoblast cell line from an FA—A patient. The SV40-transformed FA fibroblasts, GM6914, PD426, FAG326SV and PD20F, as well as Hela cells, were grown in DMEM supplemented with 15% FCS. FA cells (both lymphoblasts and fibroblasts) were functionally complemented with pMMP retroviral vectors containing the corresponding FANC cDNAs, and functional complementation was confirmed by the MMC assay (Garcia-Higuera et al., 1999, Mol.Cell.Biol. 19: 4866-4873) (Kuang et al., 2000, Blood 96: 1625-1632).

Cell Cycle Synchronization. HeLa cells, GM6914 cells, and GM6914 cells corrected with the pMMP-FANCA retrovirus were synchronized by the double thymidine block method as previously described, with minor modifications (Kupfer et al., 1997, Blood 90: 1047-1054). Briefly, cells were treated with 2 mM thymidine for 18 hours, thymidine-free media for 10 hours, and additional 2 mM thymidine for 18 hours to arrest the cell cycle at the G1/S boundary. Cells were washed twice with PBS and then released in DMEM +15% FCS and analyzed at various time intervals.

Alternatively, HeLa cells were treated with 0.5 rnM mimosine (Sigma) for 24 hours for synchronization in late G1 phase (Krude, 1999), washed twice with PBS, and released into DMEM +15% FCS. For synchronization in M phase, a nocodazole block was used (Ruffner et al., 1999, Mol.Cell.Biol. 19: 4843-4854). Cells were treated with 0.1 μg/ml nocodazole (Sigma) for 15 hours, and the non-adherent cells were washed twice with PBS and replated in DMEM +15%.

Cell Cycle Analysis. Trypsinized cells were resuspended in 0.5ml of PBS and fixed by adding 5 ml of ice-cold ethanol. Cells were next washed twice with PBS with 1% bovine serum albumin fractionV (1%BSA/PBS) (Sigma), and resuspended in 0.24 ml of 1%BSA/PBS. After adding 30 μl of 500 μg/ml propidium iodide (Sigma) in 38 mM sodium citrate (pH7.0) and 30 μl of 10 mg/ml DNase free RNaseA (Sigma), samples were incubated at 37° C.for 30 min. DNA content was measured by FACScan (Beckton Dickinson), and data were analyzed by the CellQuest and Modfit LT program (Becton Dickinson).

Generation of an anti-FANCD2 antiserum. A rabbit polyclonal antiserum against FANCD2 was generated using a GST-FANCD2 (N-terminal) fusion protein as an antigen source. A 5′ fragment was amplified by polymerase chain reaction (PCR) from the full length FANCD2 cDNA with the primers (SEQ ID NO: 95) DF4EcoRI (5′AGCCTCgaattcGTTTCCAAAAGAAGACTGTCA-3′) and (SEQ ID NO: 96) DR816Xh (5′-GGTATCctcgagTCAAGACGACAACTTATCCATCA-3′). The resulting PCR product of 841 bp, encoding the amino-terminal 272 amino acids of the FANCD2 polypeptide was digested with EcoRI/Xhol and subcloned into the EcoRIlXhoI sites of the plasmid pGEX4T-1 (Pharmacia). A GST-FANCD2 (N-terminal) fusion protein of the expected size (54 kD) was expressed in E. coli strain DH5γ, purified over glutathione-S-sepharose, and used to immunize a New Zealand White rabbit. An FANCD2-specific immune antiserum was affinity-purified by passage over an AminoLink Plus column (Pierce) loaded with GST protein and by passage over an AminoLink Plus column loaded with the GST-FANCD2 (N-terminal) fusion protein.

Generation of anti-FANCD2 MoAbs. Two anti-FANCD2 monoclonal antibodies were generated as follows. Balb/c mice were immunized with a GST-FANCD2 (N-terminal) fusion protein, which was the same fusion protein used for the generation of the rabbit polyclonal antiserum (E35) against FANCD2. Animals were boosted with immunogen for the four days before fusion, splenocytes were harvested, and hybridization with myeloma cells was performed. Hybridoma supernatants were collected and assayed using standard ELISA assay as the initial screen and immunoblot analysis of FANCD2 as the secondary screen. Two anti-human FANCD2 monoclonal antibodies (MoAbs) (FI17 and F114) were selected for further study. Hybridoma supernatants from the two positive cell lines were clarified by centrifugation. Supernatants were used as MoAbs for western blotting. MoAbs were purified using an affinity column for IgG. MoAbs were stored as 0.5 mgfml stocks in phosphate buffered saline (PBS). Anti-HA antibody (HA.11) was from Babco.

Immunoblotting. Cells were lysed with 1X sample buffer (50 mM Tris-HCl pH6.8, 86 mM 2-mercaptoethanol, 2% sodium dodecyl sulfate (SDS), boiled for 5 min, and subjected to 7.5% polyacrylamide SDS gel electrophoresis. After electrophoresis, proteins were transferred to nitrocellulose using a submerged transfer apparatus (BioRad) filled with 25 mM Tris base, 200 mM glycine, 20% methanol. After blocking with 5% non-fat dried milk in TBS-T (50 mM Tris—HCl, pH 8.0, 150 mM NaCl, 0.1% Tween 20) the membrane was incubated with the primary antibody diluted in TBS-T (1:1000 dilution for the affinity-purified anti-FANCD2 polyclonal antibody (E35) or anti-HA (HA.11), 1:200 dilution for the anti-FANCD2 mouse monoclonal antibody F117), washed extensively and incubated with the appropriate horseradish peroxidase-linked secondary antibody (Amersham). Chemiluminescence was used for detection.

Generation of DNA Damage. Gamma irradiation was delivered using a Gamma cell 40 apparatus. UV exposure was achieved using a Stratalinker (Stratagene) after gently aspirating the culture medium. For Mitomycin C treatment cells were continuously exposed to the drug for the indicated time. Hydroxyurea (Sigma) was added to a final concentration of 1 mM for 24 hours.

Detection of Monoubiquitinated FANCD2. HeLa cells (or the FA—G fibroblasts, FAG326SV) were transfected using FuGENE6 (Roche), following the manufacturer's protocol. HeLa cells were plated onto 15 cm tissue culture dishes and were transfected with 15 tg of a HA-tagged ubiquitin expression vector (pMT 123) (Treier et al., 1994, Cell 78: 787-798) per dish. Twelve hours following transfection, cells were treated with the indicated concentration of MMC (0,10,40,160 ng/ml) or the indicated dose of IR (0,5,10,10,2OGy). After 24 hour-incubation with MMC, or two hours after IR treatment, whole cell extracts were prepared in Lysis Buffer (50 mM TrisHCl pH 7.4, 150 mM NaCl, 1% (v/v) Triton X-100) supplemented with protease inhibitors (1 μg/ml leupeptin and pepstatin, 2 μg/ml aprotinin, 1 mM phenylmethylsulfonylfluoride) and phosphatase inhibitors (1 mM sodium orthovanadate, 10 mM sodium fluoride). Using the polyclonal antibody to FANCD2 (E35), immunoprecipitation (IP) was performed essentially as described (Kupfer et al., 1997) except that each IP was normalized to contain 4 mg of protein. As a negative control, preimmune serum from the same rabbit was used in IP reaction. Immunoblotting was done using anti-HA (HA.11), or anti-FANCD2 (FI17) monoclonal antibody.

Ubiquitin Aldeliyde Treatment. HeLa cells were treated with ImM hydroxyurea for 24 hours, and whole cell extracts were prepared in Lysis Buffer supplemented with protease inhibitors and phosphatase inhibitors. 200 μg of cell lysate in 67 μl of reaction with 6.7 μl of 25 μM ubiquitin aldehyde (BostonBiochem) in DMSO or with 6.7 μl of DMSO were incubated at 30° C. or at 37° C. for the indicated periods. Sixty-seven microliters of 2X sample buffer was added to each sample, and the samples were boiled for 5 min, separated by 7.5% SDS-PAGE, and immunoblotted for FANCD2 using the F117 monoclonal anti-human FANCD2 antibody.

Immunofluorescence Microscopy. Cells were fixed with 2% paraformaldehyde in PBS for 20 min., followed by permeabilization with 0.3% Triton-X-100 in PBS (10 min). After blocking in 10% goat serum, 0.1% NP-40 in PBS (blocking buffer), specific antibodies were added at the appropriate dilution in blocking buffer and incubated for 2-4 hours at room temperature. FANCD2 was detected using the affinity-purified E35 polyclonal antibody (1/100). For BRCA1 detection, we used a commercial monoclonal antibody (D-9, Santa Cruz) at 2 μg/ml. Cells were subsequently washed three times in PBS+0.1% NP-40 (10-15 min each wash) and species-specific fluorescein or Texas red-conjugated secondary antibodies (Jackson Immunoresearch) were diluted in blocking buffer (anti-mouse 1/200, anti-rabbit 1/1000) and added. After 1 hour at room temperature three more 10-15 min washes were applied and the slides were mounted in Vectashield (Vector laboratories). Images were captured on a Nikon microscope and processed using Adobe Photoshop software.

Meiotic Chromosome Staining. Surface spreads of pachytene and diplotene spermatocytes from male mice between the ages of 16 and 28 days old were prepared as described by (Peters et al., 1997). A polyclonal goat antibody to the mouse SCP3 protein was used to visualize axial elements and synaptonemal complexes in the meiotic preparations. The MI 18 mouse monoclonal antibody against mouse BRCA1 was generated by standard techniques, by immunizing mice with murine BRCA1 protein. The affinity-purified E35 rabbit polyclonal antibody was used in 1:200 dilution to detect FANCD. Antibody incubation and detection procedures were a modification of the protocol of (Moens et al., 1987, J. Cell. Biol. 105: 93-103) as described by (Keegan et al., 1996, Genes Dev. 10: 2423-2437). Combinations of donkey-anti mouse IgG-FTTC-congugated, Donkey-anti rabbit IgG-TRITC-congugated, and Donkey-anti goat IgG-Cy5-congugated secondary antibodies were used for detection (Jackson ImmunoResearch Laboratories). All preparations were counterstained with 4′, 6′diamino-2-phenylindole (DAPI, Sigma) and mounted in a DABCO (Sigma) antifade solution. The preparations were examined on a Nikon E1OOO microscope (60X CFI Plan Apochromat and lOOX CFI Plan Fluor oil-immersion objectives). Each fluorochrome (FITC, TRITC, Cy5 and DAPI) image was captured separately as an 800×1000 pixel 12-bit source image via IPLab software (Scanalytics) controlling a cooled-CCD camera (Princeton Instruments MicroMax) and the separate 12 bit grey scale images were resampled, 24-bit pseudocolored and merged using Adobe Photoshop.

### Example 2

The FA genes interact in a common cellular pathway.

Normal lymphoblasts express two isoforms of the FANCD2 protein, a short form (FANCD2-S, 155 kD) and a long form (FANCD2-L, 162 kD). FIG. 1 shows what happened when whole cell extracts were prepared from a lymphoblast line and cellular proteins were immunoprecipitated with an anti-FANCD2 antiserum. Normal wild type cells expressed two isoforms of the FANCD2 protein—a low molecular weight isoform FANCD2-S (155 kD isoform) and a high molecular weight isoform (FANCD2-L) (162 kD isoform). FANCD2-S is the primary translation product of the cloned FANCD2 CDNA. We next evaluated a large series of FA lymphoblasts and fibroblasts for expression of the FANCD2 isoforms (Table 5). Correction of these FA cell lines with the corresponding FA EDNA resulted in functional complementation and restoration of the high molecular weight isoform, FANCD2-L.

As previously described, FA cells are sensitive to the DNA crosslinking agent, MMC, and in some cases, to ionizing radiation (IR). Interestingly, FA cells from multiple complementation groups (A, C, G, and F) only expressed the FANCD2-S isoform (FIG. 1A, lanes 3,7,9,11). FA cells from complementation groups B and E also express only the FANCD2-S. Functional correction of the MMC and IR sensitivity of these FA cells with the corresponding FANC CDNA restored the FA protein complex (Garcia-Higuera et al., 1999) and restored the high molecular weight isoform (FANCD2-L) (FIG. 1A, lanes 4,8,10,12). Taken together, these results demonstrate that the FA protein complex, containing FANCA, FANCC, FANCF, and FANCG, directly or indirectly regulates the expression of the two isoforms of FANCD2. The six cloned FA genes therefore appear to interact in a common pathway.

### Example 3

The FA protein complex is required for the monoubiquitination of FANCD2

The high molecular weight isoform of FANCD2 could result from one or more mechanisms, including alternative splicing of the FANCD2 mRNA or post-translational modification(s) of the FANCD2 protein. Treatment with phosphatase did not convert FANCD2-L to FANCD2-S, demonstrating that phosphorylation alone does not account for the observed difference in their molecular mass.

In order to identify other possible post-translational modifications of FANCD2, we initially sought cellular conditions which regulate the conversion of FANCD2-S to FANCD2-L (FIG. 1 B, C). Since FA cells are sensitive to MMC and IR, we reasoned that these agents might regulate the conversion of FANCD2-S to FANCD2-L in normal cells. Interestingly, HeLa cells treated with MMC (FIG. 1B, lanes 1-6) or IR (FIG. 1C, lanes 1-6) demonstrated a dose-dependent increase in the expression of the FANCD2-L isoform.

To determine whether FANCD2-L is a ubiquitinated isoform of FANCD2-S, we transfected HeLa cells with a cDNA encoding HA-Ubiquitin (Treier et al., 1994). Cellular exposure to MMC (FIG. 1B, lanes 7-10) or IR (FIG. 1C, lanes 7-10) resulted in a dose-dependent increase in the HA-ubiquitin conjugation of FANCD2. Only the FANCD2-L isoform, and not the FANCD2-S isoform, was immunoreactive with an anti-HA antibody. Although FANCD2 was not ubiquinated in FA cells, FANCD2 ubiquination was restored upon functional complementation of these cells. Although FANCD2 was not ubiquitinated in FA cells, FANCD2 ubiquitination was restored upon functional complementation of these cells. Since the FANCD2-S and FANCD2-L isoforms differ by 7 kD, the FANCD2-L probably contains a single ubiquitin moiety (76 amino acids) covalently bound by an amide linkage to an internal lysine residue of FANCD2.

To confirm the monoubiquitination, we isolated FANCD2-L protein from HeLa cells and analyzed its tryptic fragments by mass spectrometry (Wu et al., 2000, Science 289, 11a). Ubiquitin tryptic fragments were unambiguously identified, and a site of monoubiquitination (K561 of FANCD2) was also identified. Interestingly, this lysine residue is conserved among FANCD2 sequences from human, Drosophila, and C. elegans, suggesting that the ubiquitination of this site is critical to the FA pathway in multiple organisms. Mutation of this lysine residue, FANCD2 (K561R), resulted in loss of FANCD2 monoubiquitination.

### Example 4

Formation of Nuclear Foci Containing FANCD2 requires an intact FA pathway.

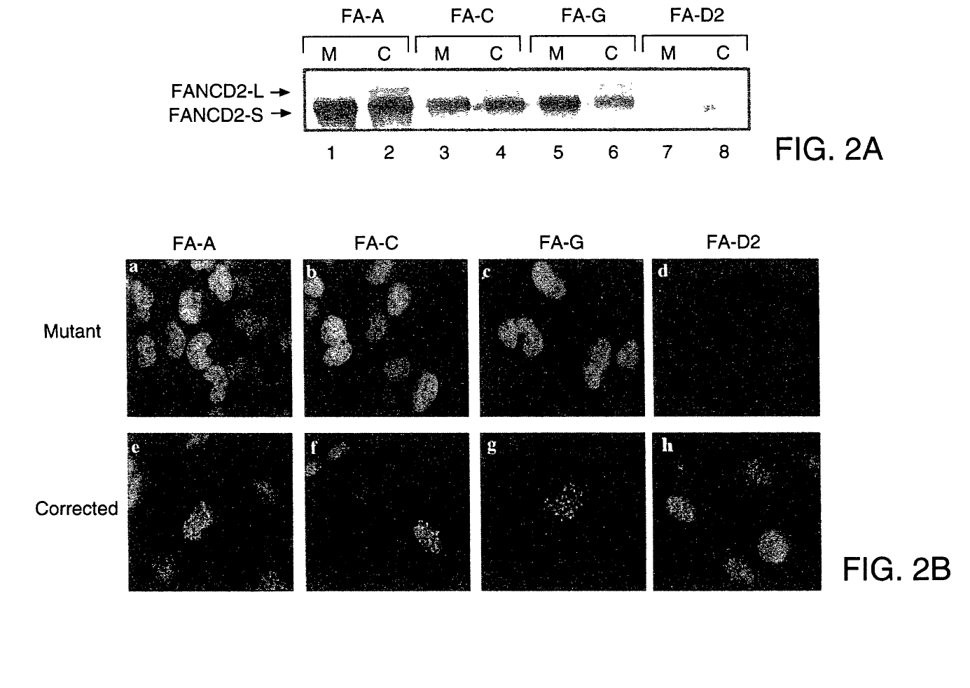

We examined the immunofluorescence pattern of the FANCD2 protein in uncorrected, MMC-sensitive FA fibroblasts and functionally-complemented fibroblasts (FIG. 2).

The corrected FA cells expressed both the FANCD2-S and FANCD2-L isoforns (FIG. 2A, lanes 2,4,6,8). The endogenous FANCD2 protein was observed exclusively in the nucleus of human cells, and no cytoplasmic staining was evident (FIG. 2B, a-h). The PD-20 (FA—D) cells have decreased nuclear immunofluorescence (FIG. 2B, d), consistent with the decreased expression of FANCD2 protein in these cells by immunoblot (FIG. 2A, lane 7). In PD-20 cells functionally-corrected with the FANCD2 gene by chromosome transfer, the FANCD2 protein stained in two nuclear patterns. Most corrected cells had a diffuse nuclear pattern of staining, and a minor fraction of cells stained for nuclear foci (see dots, panel h). Both nuclear patterns were observed with three independently-derived anti-FANCD2 antisera (1 polyclonal, 2 monoclonal antisera). FA fibroblasts from subtypes A, G, and C showed only the diffuse pattern of FANCD2 nuclear immunofluorescence. Functional complementation of these cells with the FANCA, FANCG, or FANCC cDNA, respectively, restored the MMC resistance of these cells (Table 6), and restored the nuclear foci in some cells. The presence of the high molecular weight FANCD2-L isoform therefore correlates with the presence of FANCD2 nuclear foci, suggesting that only the monoubiquitinated. FANCD2-L isoform is selectively localized to these foci.

### Example 5

The FANCD2 protein is localized to nuclear foci during S phase of the Cell Cycle

Since only a fraction of the asynchronous functionally-complemented cells contained FANCD2 nuclear foci, we reasoned that these foci might assemble at discrete times during the cell cycle. To test this hypothesis, we examined the formation of the FANCD2-L isoform and FANCD2 nuclear foci in synchronized cells (FIG. 3). HeLa cells were synchronized at the GI/S boundary, released into S phase, and analyzed for formation of the FANCD2-L isoform (FIG. 3A). The FANCD2-L isoform was expressed specifically during late GI phase and throughout S phase. Synchronized, uncomplemented FA cells (FA—A fibroblasts, GM6914) expressed normal to increased levels of FANCD2-S protein but failed to express FANCD2-L at any time during the cell cycle. Functional complementation of these FA—A cells by stable transfection with the FANCA cDNA restored S phase-specific expression of FANCD2-L. The S phase-specific expression of the FANCD2-L isoform was confirmed when HeLa cells were synchronized by other methods, such as nocodazole arrest (FIG. 3B) or mimosine exposure (FIG. 3B). Cells arrested in mitosis did not express FANCD2-L, suggesting that the FANCD2-L isoform is removed or degraded prior to cell division (FIG. 3B, mitosis). Taken together, these results demonstrate that the monoubiquitination of the FANCD2 protein is highly regulated during the cell cycle, and that this modification requires an intact FA pathway.

The cell cycle dependent expression of the FANCD2-L isoform also correlated with the formation of FANCD2 nuclear foci (FIG. 3 C). Nocodazole arrested (mitotic) cells express no FANCD2-L isoform and exhibit no FANCD2 nuclear foci (FIG. 3C, 0 hour). When these synchronized cells were allowed to traverse S phase (15 to 18 hours), an increase in FANCD2 nuclear foci was observed.

### Example 6

The FANCD2 protein is localized to nuclear foci in response to DNA damage.

We examined the accumulation of the FANCD2-L isoform and FANCD2 nuclear foci in response to DNA damage (FIG. 4). Previous studies have shown that FA cells are sensitive to agents which cause DNA interstrand crosslinks (MMC) or double strand breaks (IR) but are relatively resistant to ultraviolet light (UV) and monofunctional alkylating agents. MMC activated the conversion of FANCD2-S to FANCD2-L in asynchronous HeLa cells (FIG. 4A). Maximal conversion to FANCD2-L occurred 12-24 hours after MMC exposure, correlating with the time of maximal FANCD2 nuclear focus formation. There was an increase in FANCD2 nuclear foci corresponding to the increase in FANCD2-L. Ionizing radiation also activated a time-dependent and dose-dependent increase in FANCD2-L in HeLa cells, with a corresponding increase in FANCD2 foci (FIG. 4B). Surprisingly, ultraviolet (UV) light activated a time-dependent and dose-dependent conversion of FANCD2-S to FANCD2-L, with a corresponding increase in FANCD2 foci (FIG. 4C).

We tested the effect of DNA damage on FA cells (FIG. 4D). FA cells from multiple complementation groups (A, C, and G) failed to activate the FANCD2-L isoform and failed to activate FANCD2 nuclear foci in response to MMC or IR exposure. These data suggest that the cellular sensitivity of FA cells results, at least in part, from their failure to activate FANCD2-L and FANCD2 nuclear foci.

### Example 7

Co-localization of activated FANCD2 and BRCAl protein.

Like FANCD2, the breast cancer susceptibility protein, BRCAl, is upregulated in proliferating cells and is activated by post-translational modifications during S phase or in response to DNA damage. BRCA has a carboxy terminus 20aminoacids which contain a highly acidic HMG—like domain suggesting a possible mechanism for chromatin repair The BRCAl protein colocalizes in IR-inducible foci (IRIFs) with other proteins implicated in DNA repair, such as RAD51 or the NBS/Mrel 1/RAD50 complex. Cells with biallelic mutations in BRCAl have a defect in DNA repair and are sensitive to DNA damaging agents such as IR and MMC (Table 5). Taken together, these data suggest a possible functional interaction between the FANCD2 and BRCAl proteins. BRCA foci are large (2 mDa) multiprotein complexes including ATM and ATM substrates involved in DNA repair (BRCAl) and checkpoint functions (NBS)

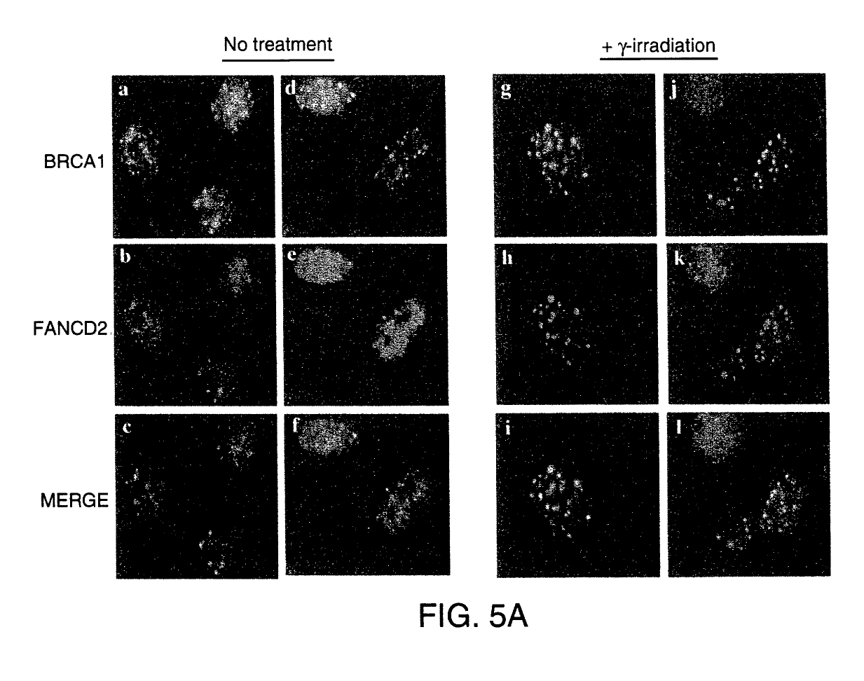

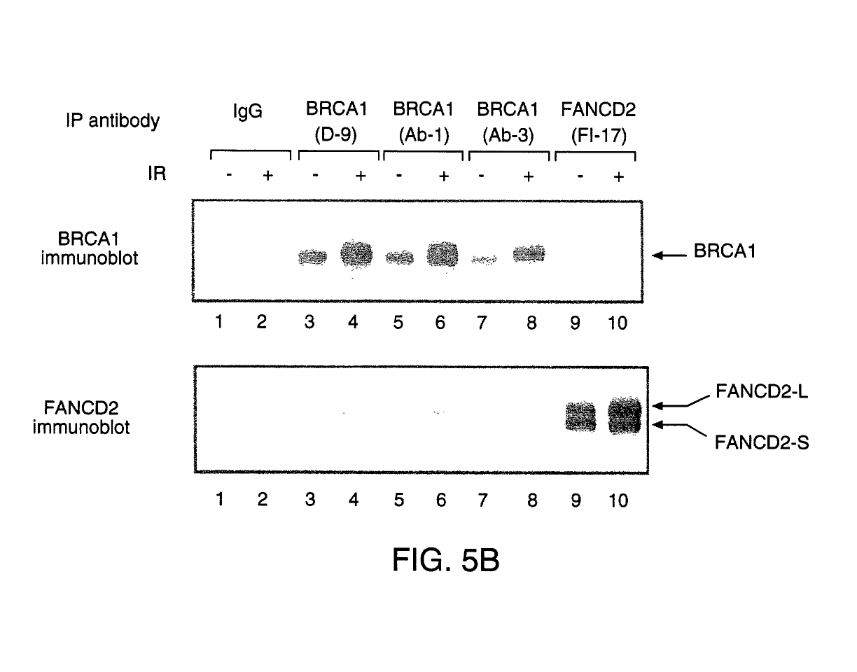

In order to determine whether the activated FANCD2 protein colocalizes with the BRCAl protein, we performed double immunolabelling of HeLa cells (FIG. 5). In the absence of ionizing radiation, approximately 30-50% of cells contained BRCA1 nuclear foci (FIG. 5A). In contrast, only rare cells traversing S phase contained FANCD2 dots (b,e). These nuclear foci were also immunoreactive with antisera to both BRCAl and FANCD2 (c,f). Following IR exposure, there was an increase in the number of cells containing nuclear foci and the number of foci per cell. These nuclear foci were larger and more fluorescent than foci observed in the absence of IR. Again, these foci contained both BRCA1 and FANCD2 protein (i, 1). An interaction of FANCD2-L and BRCA1 was further confirmed by coimmunoprecipitation of the proteins (FIG. 5B) from exponentially growing HeLa cells exposed to IR.

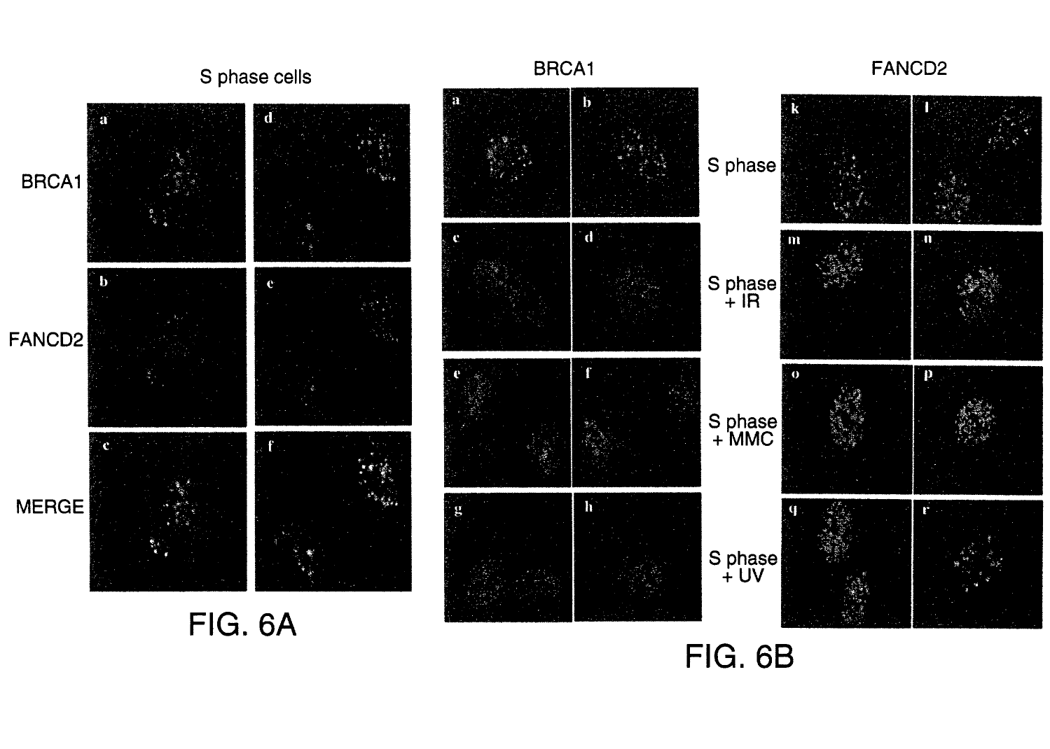

We examined the effect of BRCAl expression on the formation of FANCD2-L and nuclear foci (FIG. 6). The BRCA1 (−/−) cell line, HCC1937, expresses a mutant form of the BRCAl protein with a carboxy terminal truncation. Although these cells expressed a low level of FANCD2-L (FIG. 6A), IR failed to activate an increase in FANCD2-L levels. Also, these cells had a decreased number of IR-inducible FANCD2 foci (FIG. 6B, panels c, d). Correction of these BRCA1 (−/−) cells by stable transfection with the BRCA1 cDNA restored IR-inducible FANCD2 ubiquitination and nuclear foci (FIG. 6B, panels k, 1). These data suggest that the wild-type BRCA1 protein is required as an “organizer” for IR-inducible FANCD2 dot formation and further suggests a functional interaction between the proteins.

### Example 8

Co-localization of FANCD2 and BRCA1 on Meiotic Chromosomes.

The association of FANCD2 and BRCA1 in mitotic cells suggested that these proteins might also colocalize during meiotic prophase. Previous studies have demonstrated that the BRCA1 protein is concentrated on the unsynapsed/axial elements of human synaptonemal complexes in zygotene and pachytene spermatocytes. To test for a possible colocalization of FANCD2 and BRCA1 in meiotic cells, we examined surface spreads of late pachytene and early diplotene mouse spermatocytes for the presence of FANCD2 and BRCA1 protein (FIG. 7). We found that the rabbit polyclonal anti-FANCD2 antibody E35 specifically stained the unpaired axes of the X and Y chromosomes in late pachynema (FIG. 7a) and in diplonema (FIGS. 7d, 7e and 7g). Under the same experimental conditions, preimmune serum did not stain synaptonemal complexes (FIGS. 7b and 7c). The M118 anti-BRCA1 antibody stained the unpaired sex chromosomes in mouse pachytene and diplotene spermatocytes (FIGS. 7f and 7h). FANCD2 Ab staining of the unsynapsed axes of the sex chromosomes was interrupted, giving a beads-on-a-string appearance (FIG. 7g). A consecutive examination of 20 pachytene nuclei indicated that most (˜65%) of these anti-FANCD2 foci colocalized with regions of intense anti-BRCA1 staining, further supporting an interaction between these proteins (FIGS. 7g, 7h, and 7i). These results provide the first example of a FANC protein (activated FANCD2) which binds to chromatin.

### Example 9

Experimental protocols for obtaining and analyzing the DNA and protein sequence for FANCD2.

Northern Hybridizations. Human adult and fetal multi-tissue mRNA blots were purchased from Clontech (Palo Alto, Calif.). Blots were probed with ³²P labeled DNA from EST clone SGC34603. Standard hybridization and washing conditions were used. Equal loading was confirmed by re-hybridizing the blot with an actin cDNA probe.

Mutation Analysis. Total cellular RNA was reverse transcribed using a commercial kit (Gibco/BRL). The 5′ end section of FANCD2 was amplified from the resulting patient and control cDNA with a nested PCR protocol. The first round was performed with primers (SEQ ID NO: 97) MG471 5′-AATCGAAAACTACGGGCG-3′ and (SEQ ID NO: 98) MG457 5′-GAGAACACATGAATGAACGC-3′. The PCR product from this round was diluted 1:50 for a subsequent round using primers (SEQ ID NO: 99) MG492 5′-GGCGACGGCTTCTCGG AAGTAATTTAAG-3′ and (SEQ ID NO: 100) MG472 5′AGCGGCAGGAGGTTTATG-3′. The PCR conditions were as follows: 94° C. for 3 min, 25 cycles of 94° C. for 45 sec, 50° C. for 45 sec, 72° C. for 3 min and Smin of 72° C. at the end. The 3′ portion of the gene was amplified as described above but with primers, (SEQ ID NO: 101) MG474 5′-TGGCGGCAGACAGAAGTG-3′ and (SEQ ID NO: 102) MG475 5′-TGGCGGCAGACAGAAGTG-3′. The second round of PCR was performed with (SEQ ID NO: 103) MG491 5′-AGAGAGCCAACCTGAGCGATG-3′ and (SEQ ID NO: 104) MG476 5′-GTGCCAGACTCTGGTGGG-3′. The PCR products were gel-purified, cloned into the pT-Adv vector (Clontech) and sequenced using internal primers.

Allele specific assays: Allele specific assays were performed in the PD20 family and 290 control samples (=580 chromosomes). The PD20 family is of mixed Northern European descent and VU008 is a Dutch family. Control DNA samples were from unrelated individuals in CEPH families (n=95), samples from unrelated North American families with either ectodermal dysplasia (n=95) or Fanconi Anemia (n=94). The maternal nt376a→g mutation in the PD 20 family created a novel MspI restriction site. For genomic DNA, the assay involved amplifying genomic DNA using the primers (SEQ ID NO: 105) MG792 5′-AGGA GACACCCTTC CTATCC-3′ located in exon 4 and (SEQ ID NO: 106) MG803 5′-GAAG TTGGCAAAACAGACTG-3′which is in intron 5. The size of the PCR product was 340 bp, yielding two fragments of 283 bp and 57 bp upon MspI digestion if the mutation was present. For analysis of the reverted cDNA clones, PCR was performed using primers (SEQ ID NO: 107) MG924 5′-TGTCTTGTGAGCGTCTGCAGG-3′ and (SEQ ID NO: 108) MG753 5′-AGGTT TTGATAATGGCAGGC-3′. The paternal exon 37 mutation (R1236H1) in PD20 and exon 12 missense mutation (R302W) in VU008 were tested by allele specific oligonucleotide (ASO) hybridization (Wu, et al., 1989, DNA 8: 135-142). For the exon 12 assay, genomic DNA was amplified with primers (SEQ ID NO: 109) MG979 5′-ACTGGACTGTGCCTACCCACTATG-3′ and (SEQ ID NO: 110) MG984 5′-CCTGTGTGAGGATGAGCTCT-3′. Primers (SEQ ID NO: 171) MG818 5′-AGAGGTAGGGAAGGAAGCTAC-3′ and (SEQ ID NO: 172) MG813 5′-CCAAAGT CCACTTCTTGAAG-3′ were used for exon 37. Wild-type (SEQ ID NO: 111) (5′-TTCTCCCGAAGCTCAG-3′ for R302W and (SEQ ID NO: 112) 5′-TTTCTTCC GTGTGATGA-3′ for R1236H) and mutant SEQ ID NO: 111 (5′-TTCTCCCAAAGCTGAG-3′ R302W and SEQ ID NO: 112 5′-TTTCTTCCATGTGATGA-3′ for R1236H) oligonucleotides were end-labeled with γ³²P-[ATP] and hybridized to dot-blotted target PCR products as previously ss novel DdeI site. The wild-type PCR product digests into a 117 and 7ibp product, whereas the mutant allele yields three fragments of 56, 61 and 71bps in length. PCR in all of the above assays was performed with 50 ng of genomic DNA for 37 cycles of 94° C. for 25 sec, 50° C. for 25 sec and 72° C. for 35 sec.

Generation of an anti-FANCD2 antiserum: A rabbit polyclonal antiserum against FANCD2 was generated using a GST-FANCD2 (N-terminal) fusion protein as an antigen source. A 5′ fragment was amplified by polymerase chain reaction (PCR) from the full length FANCD2 cDNA with the primers (SEQ ID NO: 113) DF4EcoRI (5′-AGCCTCgaattcGTTTCC AAAAGAAGACTGTCA-3′) and (SEQ ID NO: 114) DR816Xh (5′-GGTATCctcgagTCAAGA CGACAACTTATCCATCA-3′). The resulting PCR product of 841 bp, encoding the amino-terminal 272 amino acids of the FANCD2 polypeptide was digested with EcoRI/XhoI and subdloned into the EcoRI/Xhol sites of the plasmid pGEX4T-1 (Pharmacia). A GST-FANCD2 (N-terminal) fusion protein of the expected size (54 kD) was expressed in E. coli strain DH5□, purified over glutathione-S-sepharose, and used to immunize a New Zealand White rabbit. An FANCD2-specific immune antiserum was affinity-purified over an AminoLink Plus column (Pierce) loaded with GST protein and over an AminoLink Plus column loaded with the GST-FANCD2 (N-terminal) fusion protein.

Immunoblotting As in Example 1.

Cell Lines and Transfections: PD20i is an immortalized and PD733 a primary FA fibroblast cell line generated by the Oregon Health Sciences Fanconi Anemia cell repository (Jakobs, et al., 1996, Somet.Cell.Mol.Genet. 22: 151-157). PD20 lymphoblasts were derived from bone marrow samples. VU008 is a lymphoblast and VU423 a fibroblast line generated by the European Fanconi Anemia Registry (EUFAR). VU423i was an immortalized line derived by transfection with SV40 T-antigen (Jakobs, et al., 1996) and telomerase (Bodnar, et al., 1998, Science 279: 349-352). The other FA cell lines have been previously described. Human fibroblasts were cultured in MEM and 20% fetal calf serum. Transformed lymphoblasts were cultured in RPMI 1640 supplemented with 15% heat-inactivated fetal calf serum.

To generate FANCD2 expression constructs, the full-length cDNA was assembled from cloned RT-PCR products in pBluescript and the absence of PCR induced mutations was confirmed by sequencing. The expression vectors pIRES-Neo, pEGFP-NI, pRevTRE and pRevTet-off were from ClonTech (Palo Alto, CA). The FANCD2 was inserted into the appropriate multi-cloning site of these vectors. Expression constructs were electroporated into cell line PD20 and a normal control fibroblast cell line, GM639 using standard conditions (van den Hoff, et al., 1992). Neomycin selection was carried out with 400 jug/ml active G418 (Gibco).

Whole cell fusions: For the whole cell fusion experiments, a PD20 cell line (PD20i) resistant to hygromycin B and deleted for the HPRT locus was used (Jakobs, et al., 1997, Somat. Cell. Mol. Genet. Vol. 23, pp. 1-7). Controls included PD 24 (primary fibroblasts from affected sibling of PD20) and PD 319i (Jakobs, et al., 1997) (immortal fibroblasts from a non-A, C, D or G FA patient). 2.5×10⁵ cells from each cell line were mixed in a T25 flask and allowed to recover for 24 hours. The cells were washed with serum-free medium and then fused with 50% PEG for 1 min. After removal of the PEG, the cells were washed 3x with serum-free medium and allowed to recover overnight in complete medium without selection. The next day, cells were split 1:10 into selective medium containing 400 μg/rml hygromycin B (Roche Molecular) and 1X HAT. After the selection was complete, hybrids were passaged once and then analyzed as described below.

Retroviral Transduction of FA—D2 cells and complementation analysis: The full length FANCD2 cDNA was subcloned into the vector, pMMP-puro (Pulsipher, et al., 1998). Retroviral supernatants were used to transduce PD20F, and puromycin resistant cells were selected. Cells were analyzed for MMC sensitivity by the crystal violet assay (Naf, et al., 1998).

Chromosome Breakage Analysis: Chromosome breakage analysis was performed by the Cytogenetics Core Lab at OHSU (Portland, Oreg.). For the analysis (Cohen, et al., 1982) cells were plated into T₂₅ flasks, allowed to recover and then treated with 300 ng/ml of DEB for two days. After treatment, the cells were exposed to colcemid for 3 hours and harvested using 0.075 M KCl and 3:1 methanol:acetic acid. Slides were stained with Wright's stain and 50-100 metaphases were scored for radials.

### Example 10

Mouse models for FA for use in screening potential therapeutic agents.

Murine models of FANCD2 can be made using homologous recombination in embryonic stem cells or targeted disruption as described in D'Andrea et al. (1997) 90:1725-1736, and Yang et al. (2001) Blood 98; 1-6. The knockout of FANCD2 locus in mice is not a lethal mutation. These knock-out animals have increased susceptibility to cancer and furthermore display other symptoms characteristic of FA. It is expected that administering certain therapeutic agents to the knock-out mice will reduce their susceptibility to cancer. Moreover, it is expected that certain established chemotherapeutic agents will be identified that are more effective for treating knock-out mice who have developed cancers as a result of the particular genetic defect and this will also be useful in treating human subjects with susceptibility to cancer or who have developed cancers as a result of a mutation in the FANCD2 locus.

We can generate experimental mice models with targeted disruptions of FANCD2 using for example the approach described by Chen et al (1996) Nat. Genet. Vol. 12, pp. 448-451, for Fancc who created a disruption in an exon of the gene, and by Whitney et al (1996) Vol. 88, pp. 49-58, who used homologous recombination to create a disruption of an exon of the gene. In both animal models, spontaneous chromosome breakage and an increase in chromosome breaks in splenic lymphocytes in response to bifunctional alkylating agents are observed. In both models, Fancd2-/-mice have germ cell defects and decreased fertility. The Fancd2 murine knockout model is useful in examining (1) the role of the Fancd2 gene in the physiologic response of hematopoietic cells to DNA damage, (2) the in vivo effects of inhibitory cytokines on FA marrow cells, and (3) the efficacy of gene therapy and (4) for screening candidate therapeutic molecules.

The availability of other FA gene disruptions will allow the generation and characterization of mice with multiple FA gene knockouts. For instance, if 2 FA genes function exclusively in the same cellular pathway, a double knockout should have the same phenotype as the single FA gene knockout.

The murine Fancd2 gene can be disrupted by replacing exons with an FRT-flanked neomycin cassette via homologous recombination in 129/SvJae embryonic stem cells. Mice homozygous for the Fancd2 mutation within a mixed genetic background of 129/Sv and C57BL can be generated following standard protocols. Mouse tail genomic DMA can be prepared as previously described and used as a template for polymerase chain reaction (PCR) genotyping.

Splenocytes can be prepared from 6-week-old mice of known Fancd2 genotype. The spleen is dissected, crushed in RPMI medium into a single-cell suspension, and filtered through a 70 itm filter. Red cells are lysed in hypotonic ammonium chloride. The remaining splenic lymphocytes are washed in phosphate-buffered saline and resuspended in RPMII/10% fetal bovine serum plus phytohemagglutinin. Cells are tested for viability by the trypan blue exclusion assay. Cells are cultured for 24 hours in media and exposed to MMC or DEB for an additional 48 hours. Alternatively, cells are cultured for 50 hours, exposed to IR (2 or 4 Gy, as indicated), and allowed to recover for 12 hours before chromosome breakage or trypan blue exclusion (viability) analysis.

Mononuclear cells can be isolated from the femurs and tibiae of 4-to 6-week-old Fancd2+/− or Fancd2 −/− mice, as previously described. A total of 2×104 cells were cultured in 1 mnL of MethoCult M343 media (StemCell Technologies, Vancouver, BC) with or without MMC treatment. Colonies are scored at day 7, when most of the colonies belong to the granulocyte-macrophage colony-forming unit or erythroid burst-forming unit lineages. Each number are averaged from duplicate plates, and the data derived from 2 independent experiments.

Lymphocytes isolated from thymus, spleen, and peripheral lymph nodes are stained for T-or B-lymphocyte surface molecules with fluorescein isothiocyanate-conjugated anti-CD3, CD4, and CD19 and PE-conjugated anti-CD8, CD44, CD 45B, immonuglubulin M, and B220 (BD PharMingen, CA). Stained cells were analyzed on a Counter Epics XL flow cytometry system.

Mice ovaries and testes were isolated and fixed in 4% paraformaldehyde and further processed by the core facility of the Department of Pathology at Massachusetts General Hospital.

### Example 11

Screening assays using antibody reagents for detecting increased cancer susceptibility in human subjects

Blood samples or tissue samples can be taken from subjects for testing for the relative amounts of FANCD2-S compared to FANCD2-L and the presence or absence of FANCD2-L. Using antibody reagents specific for FANCD2-S and FANCD2-L proteins (Example 1), positive samples can be identified on Western blots as shown in FIG. 14. Other antibody assays may be utilized such as, for example, one step migration binding banded assays described in U.S. Pat. Nos. 5,654,162 and 5,073,484. Enzyme linked immunosorbent assays (ELISA), sandwich assays, radioimmune assays and other immunodiagnostic assays known in the art may be used to determine relative binding concentrations of FANCD2-S and FANCD2-L.

The feasibility of this approach is illustrated by the following: FANCD2 Diagnostic Western Blot for Screening Human Cancer Cell Lines Human cancer cell lines were treated with or without ionizing radiation (as indicated in FIG. 14) and total cell proteins were electrophoresed, transferred to nitrocellulose and immunoblotted with the anti-FANCD2 monoclonal antibody of Example 1. Ovarian cancer cell line (TOV21G) expressed FANCD2-S but not FANCD2-L (see lanes 9,10). This cell line has a deletion of human chromosome 3p overlapping the FANCD2 gene and is hemizygous for FANCD2 and is predicted to have a mutation in the second FANCD2 allele which therefore fails to be monoubiquinated by the FA complex hence no FANCD2-L (lanes 9,10). This example demonstrates that antibody based tests are suited for determining lesions in the FANCD2 gene which lead to increased cancer susceptibility.

### Example 12

Screening assays using nucleic acid reagents for detecting increased cancer susceptibility in human subjects

Blood samples or tissue samples can be taken from subjects and screened using sequencing techniques or nucleic acid probes to determine the size and location of the genetic lesion if any in the genome of the subject. The screening method may include sequencing the entire gene or by using sets of probes or single probes to identify lesions It is expected that a single lesion may predominant in the population but that other lesions may arise throughout the gene with low frequency as is the case for other genetic conditions such as cystic fibrosis and the P53 tumor suppressor gene.

The feasibility of this approach is illustrated by the following: Peripheral blood lymphocytes are isolated from the patient using standard Ficoll-Hypaque gradients and genomic DNA is isolated from these lymphocytes. We use genomic PCR to amplify 44 exons of the human FANCD2 gene (see primer Table 7) and sequence the two FANCD2 alleles to identify mutations. Where such mutations are found, we distinguish these from benign polymorphisms by their ability to ablate the functional complementation of an FA—D2 indicator cell line.

TABLE 1
Complementation groups and responsible genes of Fanconi Anemia

| | Estimated percentage | Responsible | Chromosome | Number | Protein |
|---|---|---|---|---|---|
| Subtype | of patients | gene | location | of exons | product |
| A | 66% | FANCA | 16q24.3 | 43 | 163Kd |
| B | 4.3% | FANCB | — | — | — |
| C | 12.7% | FANCC | 9q22.3 | 14 | 63Kd |
| D1 | rare | FANCD1 | — | — | — |
| D2 | rare | FANCD2 | 3p25.13 | 44 | 155, 162kD |
| E | 12.7% | FANCE | 6p21.2-21.3 | 10 | 60kD |
| F | rare | FANCF | 11p15 | 1 | 42kD |
| G | rare | FANCG | 9p13 | 14 | 68kD |
| | | (XRCC9) | | | |

TABLE 2
Diseases of Genomic Instability

| Disease | Damaging Agent | Neoplasm | Function |
|---|---|---|---|
| FA | Cross-linking agents | Acute myeloblastic | Unknown |
| | leukemia, hepatic, | | |
| | gastrointestinal, and | | |
| | gynecological tumors | | |
| XP | UV light | Squamous cell carcinomas | Excision repair |
| AT | Ionizing radiation | Lymphoma | Afferent pathway |
| | | | to p53 |
| Bloom's | Alkylating agents | Acute lymphoblastic | Cell-cycle |
| Syndrome | | leukemia | regulation |
| Cockayne's | UV light | Basal cell carcinoma | Transcription |
| Syndrome | | repair | coupled |
| Hereditary | Unknown | Adenocarcinoma of | DNA mismatch |
| non-polyposis | | colon, ovarian cancer | repair |
| colon cancer (HNPCC) | | | |

TABLE 3
FANCD2 Sequence Alterations
Mutations

| | | |
|---|---|---|
| PD20 | nt376a→g | S126G/splice |
| | nt3707g→a | R1236H |
| VU008 | nt904c→t | R302W |
| | nt958c→t | Q320X |
| PD733 | | deletion of exon 17 |

Polymorphisms

| | |
|---|---|
| nt1122a→g | V374V |
| nt1440t→c* | H480H |
| nt1509c→t^{†} | N503N |
| nt2141c→t*^{†} | L714P |
| nt2259t→c | D753D |
| nt4098t→g*^{†} | L1366L |
| nt4453g→a^{†} | 3UTR |

*PD20 is heterozygous;
^{†}VU008 is heterozygous.

TABLE 4
Chromosome Breakage Analysis of Whole-cell Fusions.

| | DEB | MMC | % of Cells | |
|---|---|---|---|---|
| Cell line/hybrids | (ng/ml) | (ng/ml) | with radials | Phenotype |
| PD20i | 300 | | 58 | S |
| PD24p | 300 | | na* | S |
| VU423p | 300 | | na* | S |
| PD319i | 300 | | 52 | S |
| PD20i/VU423p | 300 | | 6 | R |
| PD20i/PD24p | 300 | | 30 | S |
| PD20i/PD319i | 300 | | 0 | R |
| PD20i | | 40 | 48 | S |
| VU423i | | 40 | 78 | S |
| PD20i/VU423i | | 40 | 10 | R |
| VU423i + chr. 3, | | 40 | 74 | S |
| clone 1 | | | | |
| VU423i + chr. 3, | | 40 | 68 | S |
| clone 2 | | | | |
| VU423i + chr. 3, | | 40 | 88 | S |
| clone 3 | | | | |
| PD20i + empty | 0 | 0 | 2 | |
| vector | | | | |
| | | 40 | 24 | S |
| | 200 | | 62 | S |
| PD20i + | 0 | 0 | 0 | |
| FANCD2 vector | | | | |
| | | 40 | 2 | R |
| | 200 | | 10 | R |

Groups of experiments are separated by line spaces. S, cross-linker sensitive; R, cross-linker resistant; i = immortal fibroblast line; p = primary fibroblasts
*Cell viability at this concentration was too low to score for radial formation, indicating the exquisite sensitivity of primary fibroblasts to interstrand DNA-crosslinks.

TABLE 5

| | | | | MMC | IR/ |
|---|---|---|---|---|---|
| | | | FA protein | sensi- | Bleomycin |
| | | FA | complex | tivity | sensitivity |
| Cell line/ plasmid | | Group | (1) | (2) | (3) |
| Lympho- | PD7 | Wt | + | R | R |
| blasts | HSC72 | A | − | S | |
| | HSC72 + A | A | + | R | |
| | PD4 | C | − | S | |
| | PD4 + C | C | + | R | |
| | EUFA316 | G | − | S | |
| | EUFA316 + G | G | + | R | |
| | EUFA121 | F | − | S | S |
| | EUFA121 + F | F | + | R | R |
| | PD20 | D | + | S | S |
| | PD20(R) | D | + | R | R |
| Fibro- | GM0637 | Wt | + | R | R |
| blasts | GM6914 | A | − | S | S |
| | GM694 + A | A | + | R | R |
| | PD426 | C | − | S | |
| | PD426 + C | C | + | R | |
| | FAG326SV | G | − | S | |
| | FAG326SV + G | G | + | R | |
| | PD20F | D | + | S | S |
| | 20-3-15 (+D) | D | + | R | R |
| | NBS (−/−) | NBS | + | S | S |
| | ATM (−/−) | ATM | + | S | S |
| | BRCA1 (−/−) | BRCA1 | + | S | S |

1) The presence of the FA protein complex (FANCA/FANCG/FANCC) was determined as previously described (Garcia-Higuera et al., MCB 19:4866-4873, 1999)
2) MMC sensitivity for determined by the XTT assay for lymphoblasts or by the crystal violet assay for fibroblasts.
2) IR/Bleomycin sensitivity was determined by analysis of chromosome breakage (See Materials and Methods)

TABLE 6

The Intron/Exon Junctions of FANCD

| | | | | | | | | | |
|---|---|---|---|---|---|---|---|---|---|
| | | SEQ ID | | | | SEQ ID | | | |
| Exon | Size | NO. | 5′-Donor site | Score | Intron | NO. | 3′-Acceptor site | Score | Exon |

| | | | | | | | | | |
|---|---|---|---|---|---|---|---|---|---|
| 1 | 30 | 9 | TCG gtgagtaagtg | 87 | | 52 | gtttcccgattttgctctag GAA | 85 | 2 |
| 2 | 97 | 10 | CCA gtaagtatcta | 83 | | 53 | gaaaatttttctattttcag AAA | 83 | 3 |
| 3 | 141 | 11 | TAG gtaatatttta | 78 | | 54 | ctcttcttttttctgcatag CTG | 88 | 4 |
| 4 | 68 | 12 | AAA gtatgtatttt | 81 | 159 | 55 | attttttaaatctccttaag ATA | 78 | 5 |
| 5 | 104 | 13 | CAG gtgtggagagg | 86 | 375 | 56 | gatttcttttttttttacag TAT | 91 | 6 |
| 6 | 61 | 14 | CAG gtaagactgtc | 89 | | 57 | ccctatgtcttcttttttag CCT | 86 | 7 |
| 7 | 53 | 15 | AAA gtaagtggcgt | 87 | | 58 | ttctcttcctaacattttag CAA | 80 | 5 |
| 8 | 79 | 16 | AAG gtaggcttatg | 83 | 364 | 59 | aatagtgtcttctactgcag GAC | 85 | 9 |
| 9 | 125 | 17 | CAG gtggataaacc | 80 | | 60 | tctttttctaccattcacag TGA | 86 | 10 |
| 10 | 88 | 18 | AAG gtagaaaagac | 76 | | 61 | tctgtgcttttaatttttag GTT | 85 | 11 |
| 11 | 105 | 19 | GAG gtatgctctta | 80 | 387 | 62 | ctaatatttactttctgcag GTA | 87 | 12 |
| 12 | 101 | 20 | AAG gtaaagagctc | 85 | 342 | 63 | ttcctctctgctacttgtag TTC | 84 | 13 |
| 13 | 101 | 21 | AAG gtgagatcttt | 89 | 237 | 64 | actctctcctgttttttcag GCA | 92 | 14 |
| 14 | 36 | 22 | AAG gtaatgttcat | 82 | | 65 | tgcatatttattgacaatag GTG | 73 | 15 |
| 15 | 144 | 23 | TTA gtaagtgtcag | 80 | | 66 | tctactcttccccactcaag GTT | 86 | 16 |
| 16 | 135 | 24 | CAG gtatgttgaaa | 85 | | 67 | gttgactctcccctgtatag GAA | 84 | 17 |
| 17 | 132 | 25 | AAG gtatcttattg | 77 | | 68 | tggcatcattttttccacag GGC | 89 | 18 |
| 18 | 111 | 26 | CAG gttagaggcaa | 83 | | 69 | tcttcatcatctcattgcag GAT | 87 | 19 |
| 19 | 110 | 27 | CAG gtacacgtgga | 82 | | 70 | aaaaaattctttgtttttag AAG | 79 | 20 |
| 20 | 61 | 28 | CAG gtgagttcttt | 93 | | 71 | attcttcctctttgctccag GTG | 93 | 21 |
| 21 | 120 | 29 | CTG gtaaagccaat | 81 | 445 | 72 | tgtttgtttgcttcctgaag GAA | 85 | 22 |
| 22 | 74 | 30 | AGG gtaggtattgt | 84 | 300 | 73 | attctggtttttctccgcag TGA | 88 | 23 |
| 23 | 147 | 31 | AAA gtcagtatagt | 73 | | 74 | aatttatttctccttctcag ATT | 89 | 24 |
| 24 | 101 | 32 | TAG gtatgggatga | 84 | 370 | 75 | aaatgtttgttctctctcag ATT | 86 | 25 |
| 25 | 116 | 33 | GAG gtgagcagagt | 88 | | 76 | atgtaatttgtactttgcag ATT | 82 | 26 |
| 26 | 109 | 34 | CAG gtaagagaagt | 89 | | 77 | cagcctgctgtttgtttcag TCA | 81 | 27 |
| 27 | 111 | 35 | TAG gtaagtatgtt | 90 | 272 | 78 | ttctctttttaatataaaag AAA | 73 | 28 |
| 28 | 110 | 36 | AAG gtattggaatg | 78 | | 79 | ttgctgtgacttccccatag GAG | 85 | 29 |
| 29 | 144 | 37 | GAA gtaagtgacag | 85 | | 80 | tcctttcctccatgtgacag GCT | 84 | 30 |
| 30 | 117 | 38 | AAG gttagtgtagg | 86 | | 81 | taactctgcatttattatag AAC | 80 | 31 |
| 31 | 129 | 39 | CAG gtcagaagcct | 82 | 118 | 82 | aaaatcatttttatttttag TGT | 79 | 32 |
| 32 | 119 | 40 | TTG gtaagtatgtg | 85 | | 83 | tcttaccttgacttccttag GAG | 85 | 33 |
| 33 | 111 | 41 | CAG gtgagtcataa | 90 | | 84 | tttttcttgtctccttacag CCA | 91 | 34 |
| 34 | 131 | 42 | TTG gtgatgggcct | 73 | | 85 | tttgtcttcttttctaacag CTT | 89 | 35 |
| 35 | 94 | 43 | CTG gtgagatgttt | 84 | 286 | 86 | atatttgactctcaatgcag TAT | 78 | 36 |
| 36 | 123 | 44 | CAG gtaagggagtt | 92 | | 87 | atgcttttcccgtcttctag GCA | 88 | 37 |
| 37 | 94 | 45 | CAG gtgagtaagat | 92 | | 88 | catatatttggctgccccag ATT | 81 | 38 |
| 38 | 72 | 46 | AAG gtgagtatgga | 93 | | 89 | cttgtctttcacctctccag GTA | 93 | 39 |
| 39 | 39 | 47 | AAG gtgagagattt | 89 | | 90 | agtgtgtctctcttcttcag TAT | 86 | 40 |
| 40 | 75 | 48 | CGG gtaagagctaa | 86 | | 91 | tataaacttattggttatag GAA | 77 | 41 |
| 41 | 75 | 49 | AAG gtaagaagggg | 91 | | 92 | tgttatttatttccattcag ATT | 86 | 42 |
| 42 | 147 | 50 | CAG gtaagccttgg | 91 | | 93 | cttggtccattcacatttag GGT | 80 | 43 |
| 43 | 228 | | CCA taa + 3′UTR | | 94 | | atttattctttgccccttag GAT | | 44 |
| | 96 | 51 | GAG GTATCTCTACA | | | | | | |
| 44 | 72 | | GAT tag + 3′UTR | | | | | | |

TABLE 7

PCR Primers to Amplify the 44 Exons of FANCD

| | | | | | |
|---|---|---|---|---|---|
| | Primer | SEQ ID | | Product Size | Annealing |
| Exon | Name | NO. | Primer Sequence(5′−>3′) | (bp) | Temp |

| | | | | | |
|---|---|---|---|---|---|
| 1 | MG914 | 115 | F:CTAGCACAGAACTCTGCTGC | 372 | 54 |
| | MG837 | 116 | R:CTAGCACAGAACTCTGCTGC | | |
| 2 | MG726 | 117 | F:CTTCAGCAACAGCGAGTAGTCTG | 422 | 50 |
| | MG747 | 118 | R:GATTCTCAGCACTTGAAAAGCAGG | | |
| 3 | MG773 | 119 | F:GGACACATCAGTTTTCCTCTC | 309 | 50 |
| | MG789 | 120 | R:GAAAACCCATGATTCAGTCC | | |
| 4-5 | MG816 | 121 | F:TCATCAGGCAAGAAACTTGG | 467 | 50 |
| | MG803 | 122 | R:GAAGTTGGCAAAACAGACTG | | |
| 6 | MG804 | 123 | F:GAGCCATCTGCTCATTTCTG | 283 | 50 |
| | MG821 | 124 | R:CCCGCTATTTAGACTTGAGC | | |
| 7 | MG775 | 125 | F:CAAAGTGTTTATTCCAGGAGC | 343 | 50 |
| | MG802 | 126 | R:CATCAGGGTACTTTGAACATTC | | |
| 8-9 | MG727 | 127 | F:TTGACCAGAAAGGCTCAGTTCC | 640 | 50 |
| | MG915 | 128 | R:AGATGATGCCAGAGGGTTTATCC | | |
| 10 | MG790 | 129 | F:TGCCCAGCTCTGTTCAAACC | 222 | 50 |
| | MG774 | 130 | R:AGGCAATGACTGACTGACAC | | |
| 11 | MG805 | 131 | F:TGCCCGTCTATTTTTGATGAAGC | | |
| | MG791 | 132 | R:TCTCAGTTAGTCTGGGGACAG | | |
| 12 | MG751 | 133 | F:TCATGGTAGAGAGACTGGACTGTGC | 432 | 50 |
| | MG972 | 134 | R:ACCCTGGAGCAAATGACAACC | | |
| 13-14 | MG973 | 135 | F:ATTTGCTCCAGGGTACATGGC | 555 | 50 |
| | MG974 | 132 | R:GAAAGACAGTGGGAAGGCAAGC | | |
| 15 | MG975 | 137 | F:GGGAGTGTGTGGAACAAATGAGC | 513 | 50 |
| | MG976 | 138 | R:AGTTTCTACAGGCTGGTCCTATTCC | | |
| 16 | MG755 | 139 | F:AACGTGGAATCCCATTGATGC | 379 | 48 |
| | MG730 | 140 | R:TTTCTGTGTTCCCTCCTTGC | | |
| 17 | MG794 | 141 | F:GATGGTCAAGTTACACTGGC | 382 | 50 |
| | MG778 | 142 | R:CACCTCCCACCAATTATAGTATTC | 382 | 50 |
| 18 | MG731 | 143 | F:CTATGTGTGTCTCTTTTACAGGG | 234 | 48 |
| 19 | MG779 | 145 | F:CATACCTTCTTTTGCTGTGC | 199 | 48 |
| | MG795 | 146 | R:CCACAGAAGTCAGAATCTCCACG | | |
| 20 | MG731 | 147 | F:TGTAACAAACCTGCACGTTG | 632 | 56 |
| | MG817 | 144 | R:AATGTTTCCCACCATATTGC | | |
| 21 | M0788 | 149 | F:GAGTTTGGGAAAGATTGGCAGC | 232 | 50 |
| | MG772 | 150 | R:TGTAGTAAAGCAGCTCTCATGC | | |
| 22-23 | MG733 | 151 | F:CAAGTACACTCTGCACTGCC | 652 | 50 |
| | MG758 | 152 | R:TGACTCAACTTCCCCACCAAGAG | | |
| 24-25 | MG736 | 153 | F:CTCCCTATGTACGTGGAGTAATAC | 732 | 50 |
| | MG737 | 154 | R:GGGAGTCTTGTGGGAACTAAG | | |
| 26 | MG780 | 155 | F:TTCATAGACATCTCTCAGCTCTG | | |
| | MG759 | 156 | R:GTTTTGGTATCAGGGAAAGC | | |
| 27-28 | MG760 | 157 | F:AGCCATGCTTGGAATTTTGG | 653 | 50 |
| | MG781 | 158 | R:CTCACTGGGATGTCACAAAC | | |
| 29 | MG740 | 159 | F:GGTCTTGATGTGTGACTTGTATCCC | 447 | 50 |
| | MG741 | 160 | R:CCTCAGTGTCACAGTGTTCTTTGTG | | |
| 30 | MG809 | 161 | F:CATGAAATGACTAGGACATTCC | 281 | 48 |
| | MG797 | 162 | R:CTACCCAGTGACCCAAACAC | | |
| 31-32 | MG761 | 163 | F:CGAACCCTTAGTTTCTGAGACGC | 503 | 50 |
| | MG742 | 164 | R:TCAGTGCCTTGGTGACTGTC | | |
| 33 | MG916 | 165 | F:TTGATGGTACAGACTGGAGGC | 274 | 50 |
| | MG810 | 166 | R:AAGAAAGTTGCCAATCCTGTTCC | | |
| 34 | MG762 | 167 | F:AGCACCTGAAAATAAGGAGG | 343 | 50 |
| | MG743 | 168 | R:GCCCAAAGTTTGTAAGTGTGAG | | |
| 35-36 | MG787 | 169 | F:AGCAAGAATGAGGTCAAGTTC | 590 | 50 |
| | MG806 | 170 | R:GGGAAAAACTGGAGGAAAGAACTC | | |
| 37 | MG818 | 171 | F:AGAGGTAGGGAAGGAAGCTAC | 233 | 50 |
| | MG813 | 172 | R:CTTGTGGGCAAGAAATTGAG | | |
| 38 | MG834 | 173 | F:GATGCACTGGTTGCTACATC | 275 | 50 |
| | MG836 | 174 | R:CCAGGACACTTGGTTTCTGC | | |
| 39 | MG839 | 175 | F:ACACTCCCAGTTGGAATCAG | 370 | 50 |
| | MG871 | 176 | R:CTTGTGGGCAAGAAATTGAG | | |
| 40 | MG829 | 177 | F:TGGGCTGGATGAGACTATTC | 223 | 50 |
| | MG870 | 178 | R:CCAAGGACATATCTTCTGAGCAAC | | |
| 41 | MG820 | 179 | F:TGATTATCAGCATAGGCTGG | 271 | 50 |
| | MG811 | 180 | R:CCTTACATGCCATCTGATGC | | |
| 42 | MG763 | 181 | F:CATTCAGATTCACCAGGACAC | 227 | 50 |
| | MG782 | 182 | R:CCTTACATGCCATCTGATGC | | |
| 43 | MG764 | 183 | F:AACCTTCTCCCCTATTACCC | 435 | 50 |
| 3′UTR | MG835 | 184 | R:GGAAAATGAGAGGCCTATAATGC | | |
| 44 | MG1006 | 185 | F:TGTATTCCAGAGGTCACCCAGAGC | 234 | 50 |
| 3′UTR | MG1005 | 186 | R:CCAGTAAGAAAGGCAAACAGCG | | |

We claim:

1. An isolated nucleic acid molecule, comprising: a polynucleotide selected from
(a) a nucleotide sequence encoding a polypeptide having an aminoacid sequence as shown in SEQ ID NO: 4
(b) a nucleotide sequence at least 90% identical to the nucleotide sequence of (a);
(c) a nucleotide sequence complementary to the nucleotide sequence of (b);
(d) a nucleotide sequence at least 90% identical to the nucleotide sequence shown in SEQ ID NO: 5-8, 187-188; and
(e) a nucleotide sequence complementary to the nucleotide sequence of (d).

2. An isolated nucleic acid molecule according to claim 1, wherein the polynucleotide is a DNA molecule.

3. An isolated nucleic acid molecule of claim 2, wherein the polynucleotide is cDNA.

4. An isolated nucleic acid molecule according to claim 1, wherein the polynucleotide is an RNA molecule.

5. An isolated nucleic acid molecule consisting essentially of a nucleotide sequence encoding a polypeptide having an amino acid sequence sufficiently similar to that of SEQ ID NO: 4 to retain the biological property of conversion from a short form to a long form of FANCD2 in the nucleus of a cell for facilitating DNA repair.

6. An isolated nucleic acid molecule consisting essentially of a polynucleotide having a nucleotide sequence at least 90% identical to SEQ ID NO: 9-191 or complementary to a nucleotide sequence that is at least 90% identical to SEQ ID NO: 9-191.

7. An isolated nucleic acid molecule according to claim 6, wherein the sequence is an intron/exon sequence selected from SEQ ID NO: 9-94 disclosed in Table 6.

8. An isolated nucleic acid molecule according to claim 6, wherein the sequence is a PCR primer selected from SEQ ID NO: 115-186 disclosed in Table 7.

9. A method for making a recombinant vector comprising inserting the isolated nucleic acid molecule of claim I into a vector.

10. A recombinant vector produced by the method of claim 9.

11. A method of making a recombinant host cell comprising: introducing the recombinant vector of claim 10 into a host cell.

12. A recombinant host cell produced by the method of claim 11.

13. A method of making an FA—D2 cell line, comprising:
(a) obtaining cells from a subject having a biallelic mutation in a complementation group associated with FA—D2; and
(b) infecting the cells with a transforming virus to make the FA—D2 cell line.

14. A method according to claim 13, wherein the cells are selected from fibroblasts and lymphocytes.

15. A method according to claim 13, wherein the transforming virus is selected from Epstein Barr virus and retrovirus.

16. A method according to claim 13, further comprising: characterizing the FA—D2 cell line by determining the presence of a defective FANDC2.

17. A method according to claim 16, wherein characterizing the FA—D2 cell line further comprises:
performing a diagnostic assay on the cell line, the diagnostic assay selected from (i) a Western blot or nuclear immunofluorescence using an antibody specific for FANCD2 and (ii) a DNA hybridization assay.

18. A recombinant method for producing a polypeptide, comprising: culturing a recombinant host cell wherein the host cell comprises the isolated nucleic acid molecule of claim 1.

19. An isolated polypeptide, comprising an aminoacid sequence selected from
(a) SEQ ID NO: 4;
(b) an aminoacid sequence at least 90% identical to (a);
(c) an aminoacid sequence which is encoded by a polynucleotide having a nucleotide sequence which is at least 90% identical to at least one of SEQ ID NO: 5-8, 187-188; and
(d) a polypeptide fragment of (a) -(d) wherein the fragment is at least 50 aminoacids in length.

20. An isolated polypeptide according to claim 19, encoded by a DNA having a mutation selected from nt 376 A to G, nt 3707 G to A, nt9O4C to T and nt 958C to T.

21. An isolated polypeptide according to claim 19, the polypeptide characterized by a polymorphism in DNA encoding the polypeptide, the polymorphism being selected from nt 1122A to G, nt 1440T to C, nt1509C to T, nt2141C to T, nt2259T to C, nt4098T to G, nt4453G to A.

22. An isolated polypeptide according to claim 19, the polypeptide characterized by a mutation at aminoacid 222 or aminoacid 561.

23. An antibody preparation having a binding specificity for a FAN0CD2 protein.

24. An antibody preparation according to claim 23, further comprising: monoclonal antibodies.

25. An antibody preparation according to claim 23, further comprising: polyclonal antibodies.

26. An antibody preparation according to claim 23, wherein the FANCD2 protein is FANCD2-S.

27. An antibody preparation according to claim 23, wherein the FANCD2 protein is FANCD2-L.

28. A diagnostic method for measuring FANCD2 isoforms in a biological sample, the method comprising:
(a) exposing the sample to a first antibody for forming a first complex with FANCD2-L and optionally a second antibody for forming a second complex with FANCD2-S ; and
(b) detecting with a marker, the amount of the first complex and the second complex in the sample.

29. A diagnostic method according to claim 28, wherein the sample comprises intact cells.

30. A diagnostic method according to claim 28, wherein the sample comprises lysed cells in a lysate.

31. A diagnostic method according to claim 28, wherein the biological sample is from a human subject with a susceptibility to cancer or having the initial stages of cancer.

32. A diagnostic method according to claim 31, wherein the biological sample is from a cancer in a human subject, wherein the cancer is selected from melanoma, leukemia, astocytoma, glioblastoma, lymphoma, glioma, Hodgkins lymphoma, chronic lymphocyte leukemia and cancer of the pancreas, breast, thyroid, ovary, uterus, testis, pituitary, kidney, stomach, esophagus and rectum.

33. A diagnostic method according to claim 28, wherein the biological sample is from a human fetus.

34. A diagnostic method according to claim 28, wherein the biological sample is from an adult human.

35. A diagnostic method according to claim 28, wherein the biological sample is selected from: a blood sample, a biopsy sample of tissue from the subject and a cell line.

36. A diagnostic method according to claim 28, wherein the biological sample is derived from heart, brain, placenta, liver, skeletal muscle, kidney, pancreas, spleen, thymus, prostate, testis, uterus, small intestine, colon, peripheral blood and lymphocytes.

37. A diagnostic method according to claim 28, wherein the marker is a fluorescent marker, the fluorescent marker optionally conjugated to the FANCD2-L antibody.

38. A diagnostic method according to claim 28, wherein the marker is a chemiluminescent marker, the chemiluminescent marker optionally conjugated to the FANCD2-L antibody.

39. A diagnostic method according to claim 28, further comprising: binding the first and the second complex to a third antibody conjugated to a substrate.

40. A diagnostic method according to claim 30, wherein the lysate is subjected to a separation procedure to separate FANCD2 isoforms and the seperated isoforms are identified by determining binding to the first or the second FANCD2 antibody.

41. A diagnostic test for identifying a defect in the Fanconi Anemia pathway in a cell population from a subject, comprising:
selecting an antibody to FANCD2 protein and determining whether the amount of an FAND2-L isoform is reduced in the cell population compared with amounts, in a wild type cell population; such that if the amount of the FANCD2-L protein is reduced, then determining whether an amount of any of FANCA, FANCB, FANCC, FANCD1, FANCE, FANCF or FANCG protein is altered in the cell population compared with the wild type so as to identify the defect in the Fanconi Anemia pathway in the cell population.

42. A diagnostic test according to claim 41, wherein determining the amount of an isoform relies on a separation of the FANCD2-L and FANCD2-S isoforms.

43. A diagnostic test according to claim 41, wherein the separation is achieved by gel electrophoresis.

44. A diagnostic test according to claim 41, wherein the separation is achieved by a migration binding banded test strip.

45. A screening assay for identifying a therapeutic agent, comprising:
selecting a cell population in which FAND2-L is made in reduced amounts;
exposing the cell population to individual members of a library of candidate therapeutic molecules; and
identifying those individual member molecules that cause the amount of FANCD2-L to be increased in the cell population.

46. A screening assay according to claim 45, wherein the cell population is an in vitro cell population.

47. A screening assay according to claim 45, wherein the cell population is an in vivo cell population, the in vivo population being within an experimental animal, the experimental animal having a mutant FANCD2 gene.

48. A screening assay according to claim 45, wherein the experimental animal is a knock-out mouse in which the mouse FAND2 gene has been replaced by a human mutant FANCD2 gene.

49. A screening assay according to claim 45, wherein a chemical carcinogen is added to the cell population in which FANCD2 is made in reduced amounts, to determine if any member molecules can cause the amount of FANCD2-L to be increased so as to protect the cells form the harmful effects of the chemical carcinogen.

50. An experimental animal model in which the animal FANCD2 gene has been removed and optionally replaced by a nucleic acid molecule of claim 1.

51. A method for identifying in a cell sample from a subject, a mutant FANCD2 nucleotide sequence in a suspected mutant FANCD2 allele which comprises comparing the nucleotide sequence of the suspected mutant FANCD2 allele with the wild type FANCD2 nucleotide sequence wherein a difference between the suspected mutant and the wild type sequence identifies a mutant FANCD2 nucleotide sequence in the cell sample.

52. A method according to claim 51, wherein the suspected mutant allele is a germline allele.

53. A method according to claim 51, wherein identification of a mutant FANCD2 nucleotide sequence is diagnostic for a predisposition for a cancer in the subject.

54. A method according to claim 51, wherein identification of a mutant FANCD2 nucleotide sequence is diagnostic for an increased risk of the subject bearing an offspring with Fanconi Anemia.

55. A method according to claim 51, wherein the suspected mutant allele is a somatic allele in a tumor type and identifying a mutant FANCD2 nucleotide sequence is diagnostic for the tumor type.

56. A method according to claim 51, wherein the nucleotide sequence of the wild type and the suspected mutant FANCD2 nucleotide sequence is selected from a gene, a mRNA and a cDNA made from a mRNA.

57. A method according to claim 51, wherein comparing the polynucleotide sequence of the suspected mutant FANCD2 allele with the wild type FANCD2 polynucleotide sequence, further comprises: selecting a FANCD2 probe which specifically hybridizes to the mutant FANCD2 nucleotide sequence, and detecting the presence of the mutant sequence by hybridization with the probe.

58. A method according to claim 51, wherein comparing the polynucleotide sequence of the suspected mutant FANCD2 allele with the wild type FANCD2 polynucleotide sequence, further comprises amplifying all or part of the FANCD2 gene using a set of primers specific for wild type FANCD2 DNA to produce amplified FANCD2 DNA and sequencing the FANCD2 DNA so as to identify the mutant sequence.

59. A method according to claim 5 1, wherein the mutant FANCD2 nucleotide sequence is a germline alteration in the FANCD2 allele of the human subject, the alteration selected from the alterations set forth in Table 3.

60. A method according to claim 51, wherein the mutant FANCD2 nucleotide sequence is a somatic alteration in the FANCD2 allele of the human subject, the alteration selected from the alterations set forth in Table 3.

61. A method for diagnosing a susceptibility to cancer in a subject which comprises comparing the germline sequence of the FANCD2 gene or the sequence of its mRNA in a tissue sample from the subject with the germline sequence of the FANCD2 gene or the sequence of its mRNA wherein an alteration in the germline sequence of the FANCD2 gene or the sequence of its mRNA of the subject indicates the susceptibility to the cancer.

62. A method according to claim 61, wherein an alteration is detected in a regulatory region of the FANCD2 gene.

63. A method according to claim 61, wherein the detection in the alteration in the germline sequence is determined by an assay selected from the group consisting of (a) observing shifts in electrophoretic mobility of single-stranded DNA on non-denaturing polyacrylamide gels, (b) hybridizing a FANCD2 gene probe to genomic DNA isolated from the tissue sample, (c) hybridizing an allele-specific probe to genomic DNA of the tissue sample, (d) amplifying all or part of the FANCD2 gene from the tissue sample to produce an amplified sequence and sequencing the amplified sequence, (e) amplifying all or part of the FANCD2 gene from the tissue sample using primers for a specific FANCD2 mutant allele, (f) molecular cloning all or part of the FANCD2 gene from the tissue sample to produce a cloned sequence and sequencing the cloned sequence, (g) identifying a mismatch between (i) a FANCD2 gene or a FANCD2 mRNA isolated from the tissue sample, and (ii) a nucleic acid probe complementary to the human wild-type FANCD2 gene sequence, when molecules (i) and (ii) are hybridized to each other to form a duplex, (h) amplification of FANCD2 gene sequences in the tissue sample and hybridization of the amplified sequences to nucleic acid probes which comprise wild-type FANCD2 gene sequences, (I) amplification of FANCD2 gene sequences in the tissue sample and hybridization of the amplified sequences to nucleic acid probes which comprise mutant FANCD2 gene sequences, (j) screening for a deletion mutation in the tissue sample, (k) screening for a point mutation in the tissue sample, (1) screening for an insertion mutation in the tissue sample, (m) in situ hybridization of the FANCD2 gene of the tissue sample with nucleic acid probes which comprise the FANCD2 gene.

64. A method of diagnosing a susceptibility for cancer in a subject, comprising:
(a) accessing genetic material from the subject so as to determine defective DNA repair;
(b) determining the presence of mutations in a set of genes, the set comprising FAND2 and at least one of FANCA, FANCB, FANCC, FANCD1, FANCDE, FANDF, FANDG, BRACAL and ATM; and
(c) diagnosing susceptibility for cancer from the presence of mutations in the set of genes.

65. A method for detecting a mutation in a neoplastic lesion at the FANCD2 gene in a human subject which comprises:
comparing the sequence of the FANCD2 gene or the sequence of its mRNA in a tissue sample from a lesion of the subject with the sequence of the wild-type FANCD2 gene or the sequence of its mRNA, wherein an alteration in the sequence of the FANCD2 gene or the sequence of its MRNA of the subject indicates a mutation at the FANCD2 gene of the neoplastic lesion.

66. A method according to claim 65, further comprising: determining a therapeutic protocol for treating the neoplastic lesion according to the mutation at the FANCD2 gene of the neoplastic lesion.

67. A method for confirming the lack of a FANCD2 mutation in a neoplastic lesion from a human subject which comprises comparing the sequence of the FANCD2 gene or the sequence of its mRNA in a tissue sample from a lesion of said subject with the sequence of the wild-type FANCD2 gene or the sequence of its RNA, wherein the presence of the wild-type sequence in the tissue sample indicates the lack of a mutation at the FANCD2 gene.

68. A method for determining a therapeutic protocol for a subject having a cancer, comprising:
(a) determining if a deficiency in FANCD2-L occurs in a cell sample from the subject by measuring FANCD2 isoforms according to claim 25;
(b) if a deficiency is detected in (a) , then determining whether the deficiency is a result of genetic defect in non-cancer cells; and
(c) if (b) is positive, reducing the use of a therapeutic protocol that causes increased DNA damage so as to protect normal tissue in the subject and if (b) is negative, and the deficiency is contained within a genetic defect in cancer cells only, then increasing the use of a therapeutic protocol that causes increased DNA damage so as to adversely affect the cancer cells.

69. A method of treating a FA pathway defect in a cell target, comprising: administering an effective amount of FANCD2 protein or an exogenous nucleic acid to the target.

70. A method according to claim 69, wherein the FA pathway defect is a defective FANCD2 gene and the exogenous nucleic acid vector further comprises introducing a vector according to claim 10.

71. A method according to claim 69, wherein the vector is selected from a mutant herpesvirus, a E1/E4 deleted recombinant adenovirus, a mutant retrovirus, the viral vector being defective in respect of a viral gene essential for production of infectious new virus particles.

72. A method according to claim 69, wherein the vector is contained in a lipid micelle.

73. A method for treating a patient with a defective FANCD2 gene, comprising:
providing a polypeptide described in SEQ ID No: 4, for functionally correcting a defect arising from a condition arising from the defective FANCD2 gene.

74. A cell based assay for detecting a FA pathway defect, comprising:
(a) obtaining a cell sample from a subject;
(b) exposing the cell sample to DNA damaging agents; and
(c) detecting whether FANCD2-L is upregulated, the absence of upregulation being indicative of the FA pathway defect.

75. A cell based assay according to claim 74, wherein amounts of FANCD2 are measured by an analysis technique selected from: immunoblotting for detecting nuclear foci; Western blots to detect amounts of FANCD2 isoforms and quantifying mRNA by hybridising with DNA probes.

76. A kit for use in detecting a cancer cell in a biological sample, comprising:
(a) a primer pair which binds under high stringency conditions to a sequence in the FANCD2 gene, the primer pair being selected to specifically amplify an altered nucleic acid sequence described in Table 7; and
(b) containers for each of the primers.


SEQUENCE LISTING (191 sequences)

SEQ ID NO: 1 - protein, 1451 aa, Homo Sapien
  PEPTIDE (1)...(1451): Humanfancd2
MVSKRRLSKSEDKESLTEDASKTRKQPLSKKTKKSHIANAVEENDSIFVKLLKISGIILKTGESQNQLAVDQIAFQKKLFQTLRRHPSYPKIIEEFVSGLESYIEDEDSFRNCLLSCERLQDEEASMGASYSKSLIKLLLGIDILQPAIIKTLFEKLPEYFFENRNSDEINIFRLIVSQLKWLDRVVDGKDLTTKIMQLISIAPENLQHDIITSKPEILGDSQHADVGKELSDLLIENTSLTVPILDVLSSLRLDPNFLLKVRQLVMDKLSSIRLEDLPVIIKFILHSVTAMDTLEVISELREKLDLQHCVLPSRLQASQVKLKSKGRASSSGNQESSGQSCIILLFDVIKSAIRYEKTISEAWIKAIENTASVSEHKVFDLVMLFIIVSTNTQTKKYIDRVLRNKIRSGCIQEQLLQSTFSVHYLVLKDMCSSILSLAQSLLHSLDQSIISFGSLLYKYAFKFFDTYCQQEVVGALVTHICSGNEAEVDDALDVLLELVVLNPSAMMMNAVFVQGILDYLDNISPQQIRKLFYVLSTLAFSKQNEASSHIQDDMHLVIRKQLSSTVFKYKLIGIIGAVTMAGIMAADRSESPSLTQERANLSDEQCTQVTSLLQLVHSCSEQSPQASALYYDEFANLIQHEKLDPKALEWVGHTICNDFQDAFVVDSCVVPEGDFPFPVKALYGLEEYDTQDGIAINLLPLLFSQDFAKDGGPVTSQESGGKLVSPLCLAPYFRLLRLCVERQHNGNLEEIDGLLDCPIFLTDLEPGEKLESMSAKEASFMCSLIFLTLNWFREIVNAFCQETSPENKGKVLTRLKHIVELQILLEKYLAVTPDYVPPLGNFDVETLDITPHTVTAISAKIRKKGKIERKQKTDGSKTSSSDTLSEEKNSECDPTPSHRGQLNKEFTGKEEKTSLLLHNSHAFFRELDIEVFSILHCGLVTKFILDTEMHTEATEVVQLGPPELLFLLEDLSQKLESMLTPPIARRVPFLKNKGSRNIGFSHLQQRSAQEIVHCVEQLLTPMCNHLENIHNYIQCLAAENHGVVDGPGVKVQEYHIMSSCYQRLLQIFHGLFAWSGFSQPENQNLLYSALHVLSSRLKQGEHSQPLEELLSQSVHYLQNFHQSIPSFQCALYLIRLLMVILEKSTASAQNKEKIASLARQFLCRVWPSGDKEKSNISNDQLHALLCIYLEHTESILKAIEEIAQVGVPELINSPKDASSSTFPTLTRHTPVVFFRVMMAELEKIVKKIEPGTAADSQQIHEEKLLYWNMAVRDFSILINLIKVFDSHPVLHVCLKVGRLFVEAFLKQCMPLLDISFRKHREDVLSLLETFQLDTRLLHHLCGHSKIHQDTRLTQHVPLLKKTLELLVCRVKAMLTLNNCREAFWLGNLKNRDLQGEEIKSQNSQESTADESEDDMSSQASKSKATEDGEEDEVSAGEKEQDSDESYDDSD

SEQ ID NO: 2 - protein, 1269 aa, Drosophila melanogaster
  PEPTIDE (1)...(1269): Flyfancd2
MYKQFKKRSKKPLNTIDENATIKVPRLAETTTNISVESSSGGSEENIPASQEHTQRFLSQHSVILAATLGATGESSRDIATLSRQPNNFFELVLVRAGVQLDQGDSLILACDHVPIVSKLAEIFTSASSYTDKMETFKTGLNAAMAPGSKLVQKLLTGCTVDAAGEEQIYQSQNSMFMNFLMIDFMRDACVEVLLNKIEEVAKSDRVIMGKAAIPLPLLPLMLTQLRYLTASHKVEIYSRIEVIFNRATESAKLDIIANAELILDASMHDEFVELLNTEDLFHMTTVQTLGNLSLSDRTQAKLRVRILDFATSGQCSDAILPHLIRLLLNVLKIDTDDSVRDLRRRRIKLEHITVSILEEIQHYRHILEQHITTLMNILHDFMREKNRIVSDFAKSSYSILFKIFNSIQKNILKKLLELTCDKSSPHLTTHALELLRELQRKSAKDVQNCATLLIPMLDRTSDLSLTQTRVAMDLLCHVAFPDPNLSPCLQLQEQVDMVVKKQLINSIDNIKKQGIIGCVQLIDAMARIANNGVDRDFFIASVENVDSLPDGRGKMAANLIIRTEASIGNSTESLALFFEELATVFNQRNEGTSGCELDNQFIAWACDLVTFRFQASFVTENVPETKACDSIYVLAPLFNYVRVLYKHRHQDSLESINALLGCAIVLPSFFEDDNYVSVFENFEAEQQKDILSIYFHTVNWMRVSISAFASQRDPPTRRRVLSRLGELIRIEQRMKPLLARAPVDFVAPPYQFLTNVKLSNQNQKRPGPKPAAKLNATLPEPDLTGNQPSIADFTIKVGQCKTVKTKTDFEQMYGPRERYRPMEVEIIMLLVEQKFVLNHQLEEEQMGEFLGLLELRFLLEDVVQKLEAAVLRHHDSYDADSFRPHLAKPEDFICDLLPCLHEVNNHLITLGEAIDNQLTEVSSHVYSNLDLFKDQFCYIKSCFGLCVRLFALYFAWSEWSDKSQEQLLHRILCGTLLRRKWFHYSGTLDKGGQCNIYLDELVKGFLKKSNAKSQTELLTELVKQCSILNTKDKALTSFPNFKKANFPLLFRGLCEVLIHSLSGQVSVDSRGDKLKLWESAVDLLNGLLSIVQQVEQPRNFGLFLKHSLLFLKLLLQHGMSALESIVREDPERLTRFLHELQKVTRFLHQLCCHSKSIKNTAIISYIPSLRETIETLVFRVKALLAANNCHSAFHMGNMINRDLHGDSIITPRSSFAGEENSDDELPADDTSVDETVLGDDMGITAVSVSTRPSDGSRRSKSSSRSKCF

SEQ ID NO: 3 - protein, 1286 aa, A. thaliana
  PEPTIDE (1)...(1286): Plantfancd2
MVFLSRKKPPPPPSSSSAAPSLKIPQPQKESVEFDAVEKMTAILAEVGCTLMNPYGPPCLPSDLHAFRRNLTGRLSSFSANSGERDNVGALCSVFVAGFSLYIQSPSNLRRMLSSSSTTKRDESLVRNLLLVSPIQLDIQEMLLEKLPEYFDVVTGCSLEEDVARLIINHFRTLDFIVNPHVFTDKLMQVLSICPLELKKEIIGSLPEIIGDHNCQAVVDSLEKMLQEDSAVVVAVLDSFSNLNLDDQLQEQAITVAISCIRTIDGEHMPYLLRFLLLAATPVNVRRIISQIREQLKFTGMSQPCASQNKLKGKVPAYNAEGSILHALRSSLRFKNILCQEIIKELNSLEKPRDFKVIDVWLLIDMYMNGDPVRKSIEKIFKKKVVDECIQEALLDQCIGGNKEFVKILGALVTHVGSDNKFEVSSVLEMMTALVKKYAQQLLPFSSHINGISGTCILDYLEGFTIDNLHKTYSQVYEVFSLLALSARASGDSFRSSTSNELMMIVRKQLTPSCLVLYWQVSHPDLKYKKMGLVGSLRIVSSLGDAKSVPDFSSSQVERLTNDGSLAGVDALLGCPLHLPSSKLVGSLWGRSRKKSSPSRYIMLQTGYENSLVTLPCIFCDLLNAFSSQIDEKIGCISQATVKDVTTKLLKRLRNLVFLESLLSNLITLSPQSLPELHPYSESHVEHPRKKNEKRKLDDDASQRKVSMKNNLKKSKHSDVNEKLRQPTIMDAFKKAGAVMSHSQTQLRGTPSLPSMDGSTAAGSMDENCSDNESLIVKIPQVSSALEAQPFKFRPLLPQCLSILNFPKVLSQDMGSPEYRAELPLYLYLLHDLHTKLDCLVPPGKQHPFKRGSAPGYFGRFKLVELLNQIKRLFPSLNIKLNIAISLLIRGDETSQTTWRDEFALSGNPNTSSIVVSESLVYTMVCKEVLYCFSKILTLPEFETDKSLLLNLLEAFQPTEIPVANFPDFQPFPSPGTKEYLYIGVSYFFEDILNKGNYFCSFTDDFPYPCSFSFDLAFECLLTLQLVVTSVQKYLGKVSEEANRKRNPGHFHGLVPNLHAKLGTSAEKLLRHKWVDESTDNKGLKNKVCPFVSNLRIVQFTGEMVQTILRIYLEASGSTSDLLDELACTILPQASLSKSTGEDDDARDHEFPTLCAATFRGWYKTLLEENLAILNKLVKTVSSEKRGNCQPKTTEAHLKNIQKTVNVVVSLVNLCRSHEKVTIHGMAIKYGGKYVDSFLKGSLKHKDLRGQIVSSQAYIDNEADEVEETMSGEEEPMQEDELPLTP

SEQ ID NO: 4 - protein, 1471 aa, Homo sapien
  PEPTIDE (1)...(1471): Humanfancd2
MVSKRRLSKSEDKESLTEDASKTRKQPLSKKTKKSHIANAVEENDSIFVKLLKISGIILKTGESQNQLAVDQIAFQKKLFQTLRRHPSYPKIIEEFVSGLESYIEDEDSFRNCLLSCERLQDEEASMGASYSKSLIKLLLGIDILQPAIIKTLFEKLPEYFFENRNSDEINIFRLIVSQLKWLDRVVDGKDLTTKIMQLISIAPENLQHDIITSKPEILGDSQHADVGKELSDLLIENTSLTVPILDVLSSLRLDPNFLLKVRQLVMDKLSSIRLEDLPVIIKFILHSVTAMDTLEVISELREKLDLQHCVLPSRLQASQVKLKSKGRASSSGNQESSGQSCIILLFDVIKSAIRYEKTISEAWIKAIENTASVSEHKVFDLVMLFIIVSTNTQTKKYIDRVLRNKIRSGCIQEQLLQSTFSVHYLVLKDMCSSILSLAQSLLHSLDQSIISFGSLLYKYAFKFFDTYCQQEVVGALVTHICSGNEAEVDDALDVLLELVVLNPSAMMMNAVFVQGILDYLDNISPQQIRKLFYVLSTLAFSKQNEASSHIQDDMHLVIRKQLSSTVFKYKLIGIIGAVTMAGIMAADRSESPSLTQERANLSDEQCTQVTSLLQLVHSCSEQSPQASALYYDEFANLIQHEKLDPKALEWVGHTICNDFQDAFVVDSCVVPEGDFPFPVKALYGLEEYDTQDGIAINLLPLLFSQDFAKDGGPVTSQESGGKLVSPLCLAPYFRLLRLCVERQHNGNLEEIDGLLDCPIFLTDLEPGEKLESMSAKEASFMCSLIFLTLNWFREIVNAFCQETSPENKGKVLTRLKHIVELQILLEKYLAVTPDYVPPLGNFDVETLDITPHTVTAISAKIRKKGKIERKQKTDGSKTSSSDTLSEEKNSECDPTPSHRGQLNKEFTGKEEKTSLLLHNSHAFFRELDIEVFSILHCGLVTKFILDTEMHTEATEVVQLGPPELLFLLEDLSQKLESMLTPPIARRVPFLKNKGSRNIGFSHLQQRSAQEIVHCVEQLLTPMCNHLENIHNYIQCLAAENHGVVDGPGVKVQEYHIMSSCYQRLLQIFHGLFAWSGFSQPENQNLLYSALHVLSSRLKQGEHSQPLEELLSQSVHYLQNFHQSIPSFQCALYLIRLLMVILEKSTASAQNKEKIASLARQFLCRVWPSGDKEKSNISNDQLHALLCIYLEHTESILKAIEEIAQVGVPELINSPKDASSSTFPTLTRHTPVVFFRVMMAELEKIVKKIEPGTAADSQQIHEEKLLYWNMAVRDFSILINLIKVFDSHPVLHVCLKVGRLFVEAFLKQCMPLLDISFRKHREDVLSLLETFQLDTRLLHHLCGHSKIHQDTRLTQHVPLLKKTLELLVCRVKAMLTLNNCREAFWLGNLKNRDLQGEEIKSQNSQESTADESEDDMSSQASKSKATEVSLQNPPESGTDGCILLIVLSWWSRTLPTYVYCQMLLCPFPFPP

SEQ ID NO: 5 - DNA, 5189 bp, cDNA sequence
tcgaaaactacgggcggcgacggcttctcggaagtaatttaagtgcacaagacattggtcaaaatggtttccaaaagaagactgtcaaaatctgaggataaagagagcctgacagaagatgcctccaaaaccaggaagcaaccactttccaaaaagacaaagaaatctcatattgctaatgaagttgaagaaaatgacagcatctttgtaaagcttcttaagatatcaggaattattcttaaaacgggagagagtcagaatcaactagctgtggatcaaatagctttccaaaagaagctctttcagaccctgaggagacacccttcctatcccaaaataatagaagaatttgttagtggcctggagtcttacattgaggatgaagacagtttcaggaactgccttttgtcttgtgagcgtctgcaggatgaggaagccagtatgggtgcatcttattctaagagtctcatcaaactgcttctggggattgacatactgcagcctgccattatcaaaaccttatttgagaagttgccagaatatttttttgaaaacaagaacagtgatgaaatcaacatacctcgactcattgtcagtcaactaaaatggcttgacagagttgtggatggcaaggacctcaccaccaagatcatgcagctgatcagtattgctccagagaacctgcagcatgacatcatcaccagcctacctgagatcctaggggattcccagcacgctgatgtggggaaagaactcagtgacctactgatagagaatacttcactcactgtcccaatcctggatgtcctttcaagcctccgacttgacccaaacttcctattgaaggttcgccagttggtgatggataagttgtcgtctattagattggaggatttacctgtgataataaagttcattcttcattccgtaacagccatggatacacttgaggtaatttctgagcttcgggagaagttggatctgcagcattgtgttttgccatcacggttacaggcttcccaagtaaagttgaaaagtaaaggacgagcaagttcctcaggaaatcaagaaagcagcggtcagagctgtattattctcctctttgatgtaataaagtcagctattagatatgagaaaaccatttcagaagcctggattaaggcaattgaaaacactgcctcagtatctgaacacaaggtgtttgacctggtgatgcttttcatcatctatagcaccaatactcagacaaagaagtacattgacagggtgctaagaaataagattcgatcaggctgcattcaagaacagctgctccagagtacattctctgttcattacttagttcttaaggatatgtgttcatccattctgtcgctggctcagagtttgcttcactctctagaccagagtataatttcatttggcagtctcctatacaaatatgcatttaagttttttgacacgtactgccagcaggaagtggttggtgccttagtgacccatatctgcagtgggaatgaagctgaagttgatactgccttagatgtccttctagagttggtagtgttaaacccatctgctatgatgatgaatgctgtctttgtaaagggcattttagattatctggataacatatcccctcagcaaatacgaaaactcttctatgttctcagcacactggcatttagcaaacagaatgaagccagcagccacatccaggatgacatgcacttggtgataagaaagcagctctctagcaccgtattcaagtacaagctcattgggattattggtgctgtgaccatggctggcatcatggcggcagacagaagtgaatcacctagtttgacccaagagagagccaacctgagcgatgagcagtgcacacaggtgacctccttgttgcagttggttcattcctgcagtgagcagtctcctcaggcctctgcactttactatgatgaatttgccaacctgatccaacatgaaaagctggatccaaaagccctggaatgggttgggcataccatctgtaatgatttccaggatgccttcgtagtggactcctgtgttgttccggaaggtgactttccatttcctgtgaaagcactgtacggactggaagaatacgacactcaggatgggattgccataaacctcctgccgctgctgttttctcaggactttgcaaaagatgggggtccggtgacctcacaggaatcaggccaaaaattggtgtctccgctgtgcctggctccgtatttccggttactgagactttgtgtggagagacagcataacggaaacttggaggagattgatggtctactagattgtcctatattcctaactgacctggagcctggagagaagttggagtccatgtctgctaaagagcgttcattcatgtgttctctcatatttcttactctcaactggttccgagagattgtaaatgccttctgccaggaaacatcacctgagatgaaggggaaggtgctcactcggttaaagcacattgtagaattgcaaataatcctggaaaagtacttggcagtcaccccagactatgtccctcctcttggaaactttgatgtggaaactttagatataacacctcatactgttactgctatttcagcaaaaatcagaaagaaaggaaaaatagaaaggaaacaaaaaacagatggcagcaagacatcctcctctgacacactttcagaagagaaaaattcagaatgtgaccctacgccatctcatagaggccagctaaacaaggagttcacagggaaggaagaaaagacatcattgttactacataattcccatgcttttttccgagagctggacattgaggtcttctctattctacattgtggacttgtgacgaagttcatcttagatactgaaatgcacactgaagctacagaagttgtgcaacttgggccccctgagctgcttttcttgctggaagatctctcccagaagctggagagtatgctgacacctcctattgccaggagagtcccctttctcaagaacaaaggaagccggaatattggattctcacatctccaacagagatctgcccaagaaattgttcattgtgtttttcaactgctgaccccaatgtgtaaccacctggagaacattcacaactattttcagtgtttagctgctgagaatcacggtgtagttgatggaccaggagtgaaagttcaggagtaccacataatgtcttcctgctatcagaggctgctgcagatttttcatgggctttttgcttggagtggattttctcaacctgaaaatcagaatttactgtattcagccctccatgtccttagtagccgactgaaacagggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggctgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggtatctctacaaaacccaccagagtctggcactgatggttgcattttgttaattgttctaagttggtggagcagaactttgcctacttatgtttattgtcaaatgcttctatgcccatttccattccctccataacagcttctgtgcttatataatttttgggacccagaagaaacaacgacacaatcttagaatcactcctgagtatctcgagttgtggcatttgttatagagttgacaattttctgcattatagcctctcattttccatgaattcatatctgaaaccattttagaagggagaagtcatcgaagtattttctgagtgttgagaagaatgagttaaaccatttaaacacatttgaaacatacaaaaatagaaatgtgaaagcatttggtgaaagccaaagcacagagtcagaagctgccaccttagagaactgaaataaaaatagaagttcttacgcttttttgtggtacagatgctttcgacaatttaaagaaagctaaataaaaatgtagacatggctggcgcagtggctcatgcttgtaatcctagcactttttgaggccaaggtaggaggattgcttgagtccgggagctcaaggcaaagctgcacaacataacaagaccctatctccacaaaaaaaatgaaaaataaacctgggtgcggtggctcacacctgtaatcccagcactttgggaggccgatgtgggcagatcacaaggtcaggaggtcaagaccagcctggccaacatagtgaaaccccatctctactgaaaatacaaaaattagctgggtgtggtggcacgtgcctgttatctcagctacttgggaagctga

SEQ ID NO: 6 - DNA, 5194 bp, Homo sapien
tagaatcgaaaactacgggcggcgacggcttctcggaagtaatttaagtgcacaagacattggtcaaaatggtttccaaaagaagactgtcaaaatctgaggataaagagagcctgacagaagatgcctccaaaaccaggaagcaaccactttccaaaaagacaaagaaatctcatattgctaatgaagttgaagaaaatgacagcatctttgtaaagcttcttaagatatcaggaattattcttaaaacgggagagagtcagaatcaactagctgtggatcaaatagctttccaaaagaagctctttcagaccctgaggagacacccttcctatcccaaaataatagaagaatttgttagtggcctggagtcttacattgaggatgaagacagtttcaggaactgccttttgtcttgtgagcgtctgcaggatgaggaagccagtatgggtgcatcttattctaagagtctcatcaaactgcttctggggattgacatactgcagcctgccattatcaaaaccttatttgagaagttgccagaatatttttttgaaaacaagaacagtgatgaaatcaacatacctcgactcattgtcagtcaactaaaatggcttgacagagttgtggatggcaaggacctcaccaccaagatcatgcagctgatcagtattgctccagagaacctgcagcatgacatcatcaccagcctacctgagatcctaggggattcccagcacgctgatgtggggaaagaactcagtgacctactgatagagaatacttcactcactgtcccaatcctggatgtcctttcaagcctccgacttgacccaaacttcctattgaaggttcgccagttggtgatggataagttgtcgtctattagattggaggatttacctgtgataataaagttcattcttcattccgtaacagccatggatacacttgaggtaatttctgagcttcgggagaagttggatctgcagcattgtgttttgccatcacggttacaggcttcccaagtaaagttgaaaagtaaaggacgagcaagttcctcaggaaatcaagaaagcagcggtcagagctgtattattctcctctttgatgtaataaagtcagctattagatatgagaaaaccatttcagaagcctggattaaggcaattgaaaacactgcctcagtatctgaacacaaggtgtttgacctggtgatgcttttcatcatctatagcaccaatactcagacaaagaagtacattgacagggtgctaagaaataagattcgatcaggctgcattcaagaacagctgctccagagtacattctctgttcattacttagttcttaaggatatgtgttcatccattctgtcgctggctcagagtttgcttcactctctagaccagagtataatttcatttggcagtctcctatacaaatatgcatttaagttttttgacacgtactgccagcaggaagtggttggtgccttagtgacccatatctgcagtgggaatgaagctgaagttgatactgccttagatgtccttctagagttggtagtgttaaacccatctgctatgatgatgaatgctgtctttgtaaagggcattttagattatctggataacatatcccctcagcaaatacgaaaactcttctatgttctcagcacactggcatttagcaaacagaatgaagccagcagccacatccaggatgacatgcacttggtgataagaaagcagctctctagcaccgtattcaagtacaagctcattgggattattggtgctgtgaccatggctggcatcatggcggcagacagaagtgaatcacctagtttgacccaagagagagccaacctgagcgatgagcagtgcacacaggtgacctccttgttgcagttggttcattcctgcagtgagcagtctcctcaggcctctgcactttactatgatgaatttgccaacctgatccaacatgaaaagctggatccaaaagccctggaatgggttgggcataccatctgtaatgatttccaggatgccttcgtagtggactcctgtgttgttccggaaggtgactttccatttcctgtgaaagcactgtacggactggaagaatacgacactcaggatgggattgccataaacctcctgccgctgctgttttctcaggactttgcaaaagatgggggtccggtgacctcacaggaatcaggccaaaaattggtgtctccgctgtgcctggctccgtatttccggttactgagactttgtgtggagagacagcataacggaaacttggaggagattgatggtctactagattgtcctatattcctaactgacctggagcctggagagaagttggagtccatgtctgctaaagagcgttcattcatgtgttctctcatatttcttactctcaactggttccgagagattgtaaatgccttctgccaggaaacatcacctgagatgaaggggaaggtgctcactcggttaaagcacattgtagaattgcaaataatcctggaaaagtacttggcagtcaccccagactatgtccctcctcttggaaactttgatgtggaaactttagatataacacctcatactgttactgctatttcagcaaaaatcagaaagaaaggaaaaatagaaaggaaacaaaaaacagatggcagcaagacatcctcctctgacacactttcagaagagaaaaattcagaatgtgaccctacgccatctcatagaggccagctaaacaaggagttcacagggaaggaagaaaagacatcattgttactacataattcccatgcttttttccgagagctggacattgaggtcttctctattctacattgtggacttgtgacgaagttcatcttagatactgaaatgcacactgaagctacagaagttgtgcaacttgggccccctgagctgcttttcttgctggaagatctctcccagaagctggagagtatgctgacacctcctattgccaggagagtcccctttctcaagaacaaaggaagccggaatattggattctcacatctccaacagagatctgcccaagaaattgttcattgtgtttttcaactgctgaccccaatgtgtaaccacctggagaacattcacaactattttcagtgtttagctgctgagaatcacggtgtagttgatggaccaggagtgaaagttcaggagtaccacataatgtcttcctgctatcagaggctgctgcagatttttcatgggctttttgcttggagtggattttctcaacctgaaaatcagaatttactgtattcagccctccatgtccttagtagccgactgaaacagggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggctgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggtatctctacaaaacccaccagagtctggcactgatggttgcattttgttaattgttctaagttggtggagcagaactttgcctacttatgtttattgtcaaatgcttctatgcccatttccattccctccataacagcttctgtgcttatataatttttgggacccagaagaaacaacgacacaatcttagaatcactcctgagtatctcgagttgtggcatttgttatagagttgacaattttctgcattatagcctctcattttccatgaattcatatctgaaaccattttagaagggagaagtcatcgaagtattttctgagtgttgagaagaatgagttaaaccatttaaacacatttgaaacatacaaaaatagaaatgtgaaagcatttggtgaaagccaaagcacagagtcagaagctgccaccttagagaactgaaataaaaatagaagttcttacgcttttttgtggtacagatgctttcgacaatttaaagaaagctaaataaaaatgtagacatggctggcgcagtggctcatgcttgtaatcctagcactttttgaggccaaggtaggaggattgcttgagtccgggagctcaaggcaaagctgcacaacataacaagaccctatctccacaaaaaaaatgaaaaataaacctgggtgcggtggctcacacctgtaatcccagcactttgggaggccgatgtgggcagatcacaaggtcaggagttcaagaccagcctggccaacatagtgaaaccccatctctactgaaaatacaaaaattagctgggtgtggtggcacgtgcctgttatctcagctacttgggaagctga

SEQ ID NO: 7 - DNA, 4455 bp, Homo sapien
tcgaaaactacgggcggcgacggcttctcggaagtaatttaagtgcacaagacattggtcaaaatggtttccaaaagaagactgtcaaaatctgaggataaagagagcctgacagaagatgcctccaaaaccaggaagcaaccactttccaaaaagacaaagaaatctcatattgctaatgaagttgaagaaaatgacagcatctttgtaaagcttcttaagatatcaggaattattcttaaaacgggagagagtcagaatcaactagctgtggatcaaatagctttccaaaagaagctctttcagaccctgaggagacacccttcctatcccaaaataatagaagaatttgttagtggcctggagtcttacattgaggatgaagacagtttcaggaactgccttttgtcttgtgagcgtctgcaggatgaggaagccagtatgggtgcatcttattctaagagtctcatcaaactgcttctggggattgacatactgcagcctgccattatcaaaaccttatttgagaagttgccagaatatttttttgaaaacaagaacagtgatgaaatcaacatacctcgactcattgtcagtcaactaaaatggcttgacagagttgtggatggcaaggacctcaccaccaagatcatgcagctgatcagtattgctccagagaacctgcagcatgacatcatcaccagcctacctgagatcctaggggattcccagcacgctgatgtggggaaagaactcagtgacctactgatagagaatacttcactcactgtcccaatcctggatgtcctttcaagcctccgacttgacccaaacttcctattgaaggttcgccagttggtgatggataagttgtcgtctattagattggaggatttacctgtgataataaagttcattcttcattccgtaacagccatggatacacttgaggtaatttctgagcttcgggagaagttggatctgcagcattgtgttttgccatcacggttacaggcttcccaagtaaagttgaaaagtaaaggacgagcaagttcctcaggaaatcaagaaagcagcggtcagagctgtattattctcctctttgatgtaataaagtcagctattagatatgagaaaaccatttcagaagcctggattaaggcaattgaaaacactgcctcagtatctgaacacaaggtgtttgacctggtgatgcttttcatcatctatagcaccaatactcagacaaagaagtacattgacagggtgctaagaaataagattcgatcaggctgcattcaagaacagctgctccagagtacattctctgttcattacttagttcttaaggatatgtgttcatccattctgtcgctggctcagagtttgcttcactctctagaccagagtataatttcatttggcagtctcctatacaaatatgcatttaagttttttgacacgtactgccagcaggaagtggttggtgccttagtgacccatatctgcagtgggaatgaagctgaagttgatactgccttagatgtccttctagagttggtagtgttaaacccatctgctatgatgatgaatgctgtctttgtaaagggcattttagattatctggataacatatcccctcagcaaatacgaaaactcttctatgttctcagcacactggcatttagcaaacagaatgaagccagcagccacatccaggatgacatgcacttggtgataagaaagcagctctctagcaccgtattcaagtacaagctcattgggattattggtgctgtgaccatggctggcatcatggcggcagacagaagtgaatcacctagtttgacccaagagagagccaacctgagcgatgagcagtgcacacaggtgacctccttgttgcagttggttcattcctgcagtgagcagtctcctcaggcctctgcactttactatgatgaatttgccaacctgatccaacatgaaaagctggatccaaaagccctggaatgggttgggcataccatctgtaatgatttccaggatgccttcgtagtggactcctgtgttgttccggaaggtgactttccatttcctgtgaaagcactgtacggactggaagaatacgacactcaggatgggattgccataaacctcctgccgctgctgttttctcaggactttgcaaaagatgggggtccggtgacctcacaggaatcaggccaaaaattggtgtctccgctgtgcctggctccgtatttccggttactgagactttgtgtggagagacagcataacggaaacttggaggagattgatggtctactagattgtcctatattcctaactgacctggagcctggagagaagttggagtccatgtctgctaaagagcgttcattcatgtgttctctcatatttcttactctcaactggttccgagagattgtaaatgccttctgccaggaaacatcacctgagatgaaggggaaggtgctcactcggttaaagcacattgtagaattgcaaataatcctggaaaagtacttggcagtcaccccagactatgtccctcctcttggaaactttgatgtggaaactttagatataacacctcatactgttactgctatttcagcaaaaatcagaaagaaaggaaaaatagaaaggaaacaaaaaacagatggcagcaagacatcctcctctgacacactttcagaagagaaaaattcagaatgtgaccctacgccatctcatagaggccagctaaacaaggagttcacagggaaggaagaaaagacatcattgttactacataattcccatgcttttttccgagagctggacattgaggtcttctctattctacattgtggacttgtgacgaagttcatcttagatactgaaatgcacactgaagctacagaagttgtgcaacttgggccccctgagctgcttttcttgctggaagatctctcccagaagctggagagtatgctgacacctcctattgccaggagagtcccctttctcaagaacaaaggaagccggaatattggattctcacatctccaacagagatctgcccaagaaattgttcattgtgtttttcaactgctgaccccaatgtgtaaccacctggagaacattcacaactattttcagtgtttagctgctgagaatcacggtgtagttgatggaccaggagtgaaagttcaggagtaccacataatgtcttcctgctatcagaggctgctgcagatttttcatgggctttttgcttggagtggattttctcaacctgaaaatcagaatttactgtattcagccctccatgtccttagtagccgactgaaacagggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggctgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggatggtgaagaagacgaagtaagtgctggagaaaaggagcaagatagtgatgagagttatgatgactctgattagaccccagataaattgttgcctgcttctgtgtctcaa

SEQ ID NO: 8 - DNA, 5516 bp, Mus musculus
ggaaagtcgaaaacgaagggaagcaactggcgggtccccaggaagtaatataagtggcagaagacgttagtcaaaatgatttccaaaagacgtcggctagattctgaggataaagaaaacctgacagaagatgcctccaaaaccatgcccctttccaagctggcaaagaagtctcacaattctcatgaagttgaagaaaatggcagtgtctttgtaaagcttcttaaggcttcaggactcactcttaaaactggagagaaccaaaatcagctaggtgtggatcaggtaatcttccaaaggaagctctttcaggccttgaggaagcatcctgcttatcccaaagtaatagaagagtttgttaatggcctggagtcctacactgaggacagtgagagtctcaggaactgcctgctgtcttgtgagcgcctgcaggatgaggaagccagcatgggcacattttactccaagagtctgatcaagctacttctggggattgacattttacagcctgccattatcaaaatgttatttgaaaaagtgcctcagtttctttttgaaagtgagaacagagatggaatcaacatggccagactcattatcaatcaactaaaatggctggatagaattgtggatggcaaggacctcacggcccagatgatgcagttgatcagtgttgctcccgtgaacttacagcatgacttcatcacgagccttcctgaaatcctaggggattcccagcatgctaatgtggggaaagagcttggcgagctgctggtgcagaatacttccctgactgttccaattttggatgtcttttccagtctccgacttgaccccaacttcctgtccaagatccgccagttggtgatgggcaagctgtcatctgtccgtctagaggatttccctgtgattgtaaagttccttcttcattctgtaacagacaccacttcccttgaggtcattgccgagcttcgggagaacttgaacgtccagcagtttattttgccgtcacgaattcaggcttcccaaagcaaattgaaaagtaaaggactagcaagctcttcaggaaatcaagagaacagtgataaagactgtattgttcttgtctttgatgtaataaagtcagccattagatatgagaaaaccatttcagaggcctggtttaaggcaattgaacgcattgagtccgcggctgaacataaggctttggacgtggtcatgctgctcatcatctacagcaccagcacgcagaccaagaagggcgtggagaagctgctgagaaacaagattcagtcagactgcattcaagaacagctgcttgacagtgcgttctctacacattacctggttcttaaggatatttgcccatctattcttttgctggctcagactttgtttcactctcaagaccagaggatcattttgtttggcagtcttctgtacaaatatgcttttaagttttttgatacttactgccagcaggaagtggttggtgccctagtcacccatgtctgcagtgggactgaggctgaagtcgacactgcactggatgtcctcctggagctgattgtgctaaacgcctctgctatgaggctcaatgctgcttttgttaagggcatcttagattatttggaaaatatgtcccctcagcaaatacgaaaaatcttctgtattctcagcactcttgcatttagccaacagcccggaaccagcaaccatatccaggacgacatgcacctggtgatccggaagcagctctctagcactgtgttcaagtacaagctcattgggatcattggtgcagtcaccatggccggcatcatggcggaagacagaagtgtaccatctaactcatcccagaggagcgccaatgtgagcagtgagcagcgcacacaggtgacttctttgctacaactagttcattcttgcactgagcactctccttgggcctcttctctgtattatgatgaatttgccaacctgatccaagaaaggaagttggctccaaaaaccttggagtgggttgggcagaccatcttcaatgatttccaagatgcctttgtggtagacttctgtgctgctccagagggtgactttccatttcctgtgaaagcgctctatggactggaagagtacagcactcaagacggcattgtcatcaacctcctgccgctgttctatcaggaatgtgcaaaagatgccagtcgagcgacatcacaagaatcgagccagagatcaatgtcttctttgtgcctggcttcccatttccggctgctgagactttgcgtggcaagacaacatgatggaaacttggatgagatcgatggtctcttagattgtcccctgttcctccctgacctggaacctggagagaaactggagtccatgtctgctaaagaccgttcgcttatgtgttcgctcacattcctaactttcaactggttccgagaggttgtgaatgccttctgccaacaaacatctcctgagatgaagggcaaggttcttagtcggctaaaggaccttgtagaacttcagggaatcctagagaagtacttggcagtcatcccagactatgttccgcctttcgcaagcgttgacttggacactttagatatgatgcctaggagcagttctgctgttgcagcaaaaaacagaaacaagggaaagacggggggaaagaaacaaaaagctgatagcaacaaagcatcctgttcggacacacttctaacagaagacacttcagagtgtgacatggcgccatctgggagaagccacgtagacaaggagtccacagggaaggaaggaaagacgtttgtgtcactgcagaattaccgcgcttttttccgagagctggacattgaggtcttctctattctacattctggacttgtgaccaagttcatcttagacactgaaatgcacactgaagctacagaggtcgtacagctggggcctgctgagctgctcttcttgctggaagatctttcccagaagctagagaatatgctgactgctccttttgccaagagaatctgctgctttaagaataaaggaaggcagaatattggcttctcacatcttcatcagagatctgtccaggacattgtgcactgtgtggttcagctgctaaccccgatgtgtaaccatctggagaacattcacaacttctttcagtgcttaggtgctgagcatctcagtgcagatgacaaggcgagagcgacagctcaggagcagcacaccatggcctgctgctaccagaagctgctgcaggtcttgcacgcgctctttgcgtggaagggatttactcaccaatcaaagcaccgcctcctgcactcagcccttgaggtcctctcgaaccgactaaagcagatggaacaggaccagcccttggaggaactggtcagccagagcttcagttacttgcagaacttccaccatagtgttcccagtttccagtgtggtctctaccttctcagacttctgatggcccttctggagaagtctgcagtacctaaccagaagaaagaaaaacttgcctctctggccaaacagctgctttgccgagcatggcctcatggggaaaaagagaagaaccccacttttaatgaccacctgcatgatgtgctttacatctacttggagcacacagacaatgttctgaaggccatagaggagatyactggtgttggtgtcccagaactggtcagtgctccgaaagacgccgcctcctctacattccctacgttgaccgrgcacacctttgtcatattcttccgtgtgatgatggctgaactcgagaagacggtgaagggtctycaggctggcacagcagcagattcgcagcaggttcacgaagagaagctcctctattkgaacatggctgtccgagatttcagyatccttytcaatctgatgaaagtatttgacagttatcctgttctgcatgtgtgtttaaagtatggccgtcgctttgtggaggcatttctgaagcaatgtatgccactcctcgacttcagctttagaaagcatcgggaagatgttctgagcttgctgcaaacccttcagttgaacacgaggctacttcatcacctttgtggacactccaagattcgccaggacacaagactcaccaagcaygtgcctttactcaaaaagtcactggaactgttagtttgcagagtcaaagccatgcttgtcctcaacaactgtagagaggctttctggttgggtactctcaaaaaccgagacttacagggtgaagaaattatttcccaggatccctcttcctcagagagcaatgcagaggacagtgaggatggcgtgacatctcacgtctccaggaacagagcaacagaggatggggaagatgaagcaagtgatgaacagaaggaccaggacagtgatgaaagtgacgacagctccagttagagccgagtggcatggctgccctgctcacctctgacagactctcatctctttggggtttgaagtcagatgtctgtttttctagtcagaagcatcctgtttgtccatcaagaaggggtgtttatttaattccccagtgggtttcacaggttgtctaacctccaggtccctggttcaggagtccagtgtagcatccatcgttgactaggaygaacatggctgggctgcagtgcagtkcagtgcaggtgccctagctgggccttggggttttgaaactaaaatttaggcttataatagctttgtaaataaatctgtttcagagttttgcctcagctacctttttcctcactttagatgtgattattcaaggatctcattattcaaggattaggtaatattgagttgaggtttgtgcaatcgtactggtggcctaaaagtatgttccgtactgttatcttcctggaggaatgacccaactttcttatcaatgatcaagtgtttggtttggtctgtgtcagggtctctttacatagtcctggctggtgtgttattagatatgttgaccaggagggtcttgaacattacttttgaattttaaacatttttgtacatatgtgtatgggcatatatgtgccactgtgcatatgtgtaggtcagaggatagcttatgggagtgagctctctccttccaccatgtgggttccagggttcaaactctagaccttcacctgctcagccaccttacccttttaaaatgtttggttattaatatataaaaggaaggaagacaacatcaaacatgtgctggctttgtatgtatatatagtttttatttccacattaatttgaattatgcctataatatatttgtaataatcatacaaaataattgtaatttattagaaatagaacatcaggagttaaaataggggattcttctgtcttctgccaggaagcccagtctcagagatgctgccaggctcttcctcgctgtgccattaagattatttaatttttgttaatatttttactcataccggtattaaagttatgttttgttggaaaaaaaaaaaaaaaaaaaaaaaaaaaaa

SEQ ID NO: 9 - DNA, 14 bp, Homo sapien
  Intron/Exon Junction of FANCD
tcggtgagtaagtg

SEQ ID NO: 10 - DNA, 14 bp, Homo sapien
  Intron/Exon Junctions of FANCD
ccagtaagtatcta

SEQ ID NO: 11 - DNA, 14 bp, Homo sapien
  Intron/Exon Junctions of FANCD
taggtaatatttta

SEQ ID NO: 12 - DNA, 14 bp, Homo sapien
  Intron/Exon Junctions of FANCD
aaagtatgtatttt

SEQ ID NO: 13 - DNA, 14 bp, Homo sapien
  Intron/Exon Junctions of FANCD
caggtgtggagagg

SEQ ID NO: 14 - DNA, 14 bp, Homo sapien
  Intron/Exon Junctions of FANCD
caggtaagactgtc

SEQ ID NO: 15 - DNA, 14 bp, Homo sapien
  Intron/Exon Junctions of FANCD
aaagtaagtggcgt

SEQ ID NO: 16 - DNA, 14 bp, Homo sapien
  Intron/Exon Junctions of FANCD
aaggtaggcttatg

SEQ ID NO: 17 - DNA, 14 bp, Homo sapien
  Intron/Exon Junctions of FANCD
caggtggataaacc

SEQ ID NO: 18 - DNA, 14 bp, Homo sapien
  Intron/Exon Junctions of FANCD
aaggtagaaaagac

SEQ ID NO: 19 - DNA, 14 bp, Homo sapien
  Intron/Exon Junctions of FANCD
gaggtatgctctta

SEQ ID NO: 20 - DNA, 14 bp, Homo sapien
  Intron/Exon Junctions of FANCD
aaggtaaagagctc

SEQ ID NO: 21 - DNA, 14 bp, Homo sapien
  Intron/Exon Junctions of FANCD
aaggtgagatcttt

SEQ ID NO: 22 - DNA, 14 bp, Homo sapien
  Intron/Exon Junctions of FANCD
aaggtaatgttcat

SEQ ID NO: 23 - DNA, 14 bp, Homo sapien
  Intron/Exon Junctions of FANCD
ttagtaagtgtcag

SEQ ID NO: 24 - DNA, 14 bp, Homo sapien
  Intron/Exon Junctions of FANCD
caggtatgttgaaa

SEQ ID NO: 25 - DNA, 14 bp, Homo sapien
  Intron/Exon Junctions of FANCD
aaggtatcttattg

SEQ ID NO: 26 - DNA, 14 bp, Homo sapien
  Intron/Exon Junctions of FANCD
caggttagaggcaa

SEQ ID NO: 27 - DNA, 14 bp, Homo sapien
  Intron/Exon Junctions of FANCD
caggtacacgtgga

SEQ ID NO: 28 - DNA, 14 bp, Homo sapien
  Intron/Exon Junctions of FANCD
caggtgagttcttt

SEQ ID NO: 29 - DNA, 14 bp, Homo sapien
  Intron/Exon Junctions of FANCD
ctggtaaagccaat

SEQ ID NO: 30 - DNA, 14 bp, Homo sapien
  Intron/Exon Junctions of FANCD
agggtaggtattgt

SEQ ID NO: 31 - DNA, 14 bp, Homo sapien
  Intron/Exon Junctions of FANCD
aaagtcagtatagt

SEQ ID NO: 32 - DNA, 14 bp, Homo sapien
  Intron/Exon Junctions of FANCD
taggtatgggatga

SEQ ID NO: 33 - DNA, 14 bp, Homo sapien
  Intron/Exon Junctions of FANCD
gaggtgagcagagt

SEQ ID NO: 34 - DNA, 14 bp, Homo sapien
  Intron/Exon Junctions of FANCD
caggtaagagaagt

SEQ ID NO: 35 - DNA, 14 bp, Homo sapien
  Intron/Exon Junctions of FANCD
taggtaagtatgtt

SEQ ID NO: 36 - DNA, 14 bp, Homo sapien
  Intron/Exon Junctions of FANCD
aaggtattggaatg

SEQ ID NO: 37 - DNA, 14 bp, Homo sapien
  Intron/Exon Junctions of FANCD
gaagtaagtgacag

SEQ ID NO: 38 - DNA, 14 bp, Homo sapien
  Intron/Exon Junctions of FANCD
aaggttagtgtagg

SEQ ID NO: 39 - DNA, 14 bp, Homo sapien
  Intron/Exon Junctions of FANCD
caggtcagaagcct

SEQ ID NO: 40 - DNA, 14 bp, Homo sapien
  Intron/Exon Junctions of FANCD
ttggtaagtatgtg

SEQ ID NO: 41 - DNA, 14 bp, Homo sapien
  Intron/Exon Junctions of FANCD
caggtgagtcataa

SEQ ID NO: 42 - DNA, 14 bp, Homo sapien
  Intron/Exon Junctions of FANCD
ttggtgatgggcct

SEQ ID NO: 43 - DNA, 14 bp, Homo sapien
  Intron/Exon Junctions of FANCD
ctggtgagatgttt

SEQ ID NO: 44 - DNA, 14 bp, Homo sapien
  Intron/Exon Junctions of FANCD
caggtaagggagtt

SEQ ID NO: 45 - DNA, 14 bp, Homo sapien
  Intron/Exon Junctions of FANCD
caggtgagtaagat

SEQ ID NO: 46 - DNA, 14 bp, Homo sapien
  Intron/Exon Junctions of FANCD
aaggtgagtatgga

SEQ ID NO: 47 - DNA, 14 bp, Homo sapien
  Intron/Exon Junctions of FANCD
aaggtgagagattt

SEQ ID NO: 48 - DNA, 14 bp, Homo sapien
  Intron/Exon Junctions of FANCD
cgggtaagagctaa

SEQ ID NO: 49 - DNA, 14 bp, Homo sapien
  Intron/Exon Junctions of FANCD
aaggtaagaagggg

SEQ ID NO: 50 - DNA, 14 bp, Homo sapien
  Intron/Exon Junctions of FANCD
caggtaagccttgg

SEQ ID NO: 51 - DNA, 14 bp, Homo sapien
  Intron/Exon Junctions of FANCD
gaggtatctctaca

SEQ ID NO: 52 - DNA, 23 bp, Homo sapien
  Intron/Exon Junctions of FANCD
gtttcccgattttgctctaggaa

SEQ ID NO: 53 - DNA, 23 bp, Homo sapien
  Intron/Exon Junctions of FANCD
gaaaatttttctattttcagaaa

SEQ ID NO: 54 - DNA, 23 bp, Homo sapien
  Intron/Exon Junctions of FANCD
ctcttcttttttctgcatagctg

SEQ ID NO: 55 - DNA, 23 bp, Homo sapien
  Intron/Exon Junctions of FANCD
attttttaaatctccttaagata

SEQ ID NO: 56 - DNA, 23 bp, Homo sapien
  Intron/Exon Junctions of FANCD
gatttcttttttttttacagtat

SEQ ID NO: 57 - DNA, 23 bp, Homo sapien
  Intron/Exon Junctions of FANCD
ccctatgtcttcttttttagcct

SEQ ID NO: 58 - DNA, 23 bp, Homo sapien
  Intron/Exon Junctions of FANCD
ttctcttcctaacattttagcaa

SEQ ID NO: 59 - DNA, 23 bp, Homo sapien
  Intron/Exon Junctions of FANCD
aatagtgtcttctactgcaggac

SEQ ID NO: 60 - DNA, 23 bp, Homo sapien
  Intron/Exon Junctions of FANCD
tctttttctaccattcacagtga

SEQ ID NO: 61 - DNA, 23 bp, Homo sapien
  Intron/Exon Junctions of FANCD
tctgtgcttttaatttttaggtt

SEQ ID NO: 62 - DNA, 23 bp, Homo sapien
  Intron/Exon Junctions of FANCD
ctaatatttactttctgcaggta

SEQ ID NO: 63 - DNA, 23 bp, Homo sapien
  Intron/Exon Junctions of FANCD
ttcctctctgctacttgtagttc

SEQ ID NO: 64 - DNA, 23 bp, Homo sapien
  Intron/Exon Junctions of FANCD
actctctcctgttttttcaggca

SEQ ID NO: 65 - DNA, 23 bp, Homo sapien
  Intron/Exon Junctions of FANCD
tgcatatttattgacaataggtg

SEQ ID NO: 66 - DNA, 23 bp, Homo sapien
  Intron/Exon Junctions of FANCD
tctactcttccccactcaaggtt

SEQ ID NO: 67 - DNA, 23 bp, Homo sapien
  Intron/Exon Junctions of FANCD
gttgactctcccctgtataggaa

SEQ ID NO: 68 - DNA, 23 bp, Homo sapien
  Intron/Exon Junctions of FANCD
tggcatcattttttccacagggc

SEQ ID NO: 69 - DNA, 23 bp, Homo sapien
  Intron/Exon Junctions of FANCD
tcttcatcatctcattgcaggat

SEQ ID NO: 70 - DNA, 23 bp, Homo sapien
  Intron/Exon Junctions of FANCD
aaaaaattctttgtttttagaag

SEQ ID NO: 71 - DNA, 23 bp, Homo sapien
  Intron/Exon Junctions of FANCD
attcttcctctttgctccaggtg

SEQ ID NO: 72 - DNA, 23 bp, Homo sapien
  Intron/Exon Junctions of FANCD
tgtttgtttgcttcctgaaggaa

SEQ ID NO: 73 - DNA, 23 bp, Homo sapien
  Intron/Exon Junctions of FANCD
attctggtttttctccgcagtga

SEQ ID NO: 74 - DNA, 23 bp, Homo sapien
  Intron/Exon Junctions of FANCD
aatttatttctccttctcagatt

SEQ ID NO: 75 - DNA, 23 bp, Homo sapien
  Intron/Exon Junctions of FANCD
aaatgtttgttctctctcagatt

SEQ ID NO: 76 - DNA, 23 bp, Homo sapien
  Intron/Exon Junctions of FANCD
atgtaatttgtactttgcagatt

SEQ ID NO: 77 - DNA, 23 bp, Homo sapien
  Intron/Exon Junctions of FANCD
cagcctgctgtttgtttcagtca

SEQ ID NO: 78 - DNA, 23 bp, Homo sapien
  Intron/Exon Junctions of FANCD
ttctctttttaatataaaagaaa

SEQ ID NO: 79 - DNA, 23 bp, Homo sapien
  Intron/Exon Junctions of FANCD
ttgctgtgacttccccataggag

SEQ ID NO: 80 - DNA, 23 bp, Homo sapien
  Intron/Exon Junctions of FANCD
tcctttcctccatgtgacaggct

SEQ ID NO: 81 - DNA, 23 bp, Homo sapien
  Intron/Exon Junctions of FANCD
taactctgcatttattatagaac

SEQ ID NO: 82 - DNA, 23 bp, Homo sapien
  Intron/Exon Junctions of FANCD
aaaatcatttttatttttagtgt

SEQ ID NO: 83 - DNA, 23 bp, Homo sapien
  Intron/Exon Junctions of FANCD
tcttaccttgacttccttaggag

SEQ ID NO: 84 - DNA, 23 bp, Homo sapien
  Intron/Exon Junctions of FANCD
tttttcttgtctccttacagcca

SEQ ID NO: 85 - DNA, 23 bp, Homo sapien
  Intron/Exon Junctions of FANCD
tttgtcttcttttctaacagctt

SEQ ID NO: 86 - DNA, 23 bp, Homo sapien
  Intron/Exon Junctions of FANCD
atatttgactctcaatgcagtat

SEQ ID NO: 87 - DNA, 23 bp, Homo sapien
  Intron/Exon Junctions of FANCD
atgcttttcccgtcttctaggca

SEQ ID NO: 88 - DNA, 23 bp, Homo sapien
  Intron/Exon Junctions of FANCD
catatatttggctgccccagatt

SEQ ID NO: 89 - DNA, 23 bp, Homo sapien
  Intron/Exon Junctions of FANCD
cttgtctttcacctctccaggta

SEQ ID NO: 90 - DNA, 23 bp, Homo sapien
  Intron/Exon Junctions of FANCD
agtgtgtctctcttcttcagtat

SEQ ID NO: 91 - DNA, 23 bp, Homo sapien
  Intron/Exon Junctions of FANCD
tataaacttattggttataggaa

SEQ ID NO: 92 - DNA, 23 bp, Homo sapien
  Intron/Exon Junctions of FANCD
tgttatttatttccattcagatt

SEQ ID NO: 93 - DNA, 23 bp, Homo sapien
  Intron/Exon Junctions of FANCD
cttggtccattcacatttagggt

SEQ ID NO: 94 - DNA, 23 bp, Homo sapien
  Intron/Exon Junctions of FANCD
atttattctttgccccttaggat

SEQ ID NO: 95 - DNA, 33 bp, DF4EcoRI
agcctcgaattcgtttccaaaagaagactgtca

SEQ ID NO: 96 - DNA, 35 bp, DR816Xh
ggtatcctcgagtcaagacgacaacttatccatca

SEQ ID NO: 97 - DNA, 18 bp, MG471
aatcgaaaactacgggcg

SEQ ID NO: 98 - DNA, 20 bp, MG457
gagaacacatgaatgaacgc

SEQ ID NO: 99 - DNA, 28 bp, MG492
ggcgacggcttctcggaagtaatttaag

SEQ ID NO: 100 - DNA, 18 bp, MG472
agcggcaggaggtttatg

SEQ ID NO: 101 - DNA, 18 bp, MG474
tggcggcagacagaagtg

SEQ ID NO: 102 - DNA, 18 bp, MG475
tggcggcagacagaagtg

SEQ ID NO: 103 - DNA, 21 bp, MG491
agagagccaacctgagcgatg

SEQ ID NO: 104 - DNA, 18 bp, MG476
gtgccagactctggtggg

SEQ ID NO: 105 - DNA, 20 bp, MG792
aggagacacccttcctatcc

SEQ ID NO: 106 - DNA, 20 bp, MG803
gaagttggcaaaacagactg

SEQ ID NO: 107 - DNA, 21 bp, MG924
tgtcttgtgagcgtctgcagg

SEQ ID NO: 108 - DNA, 20 bp, MG753
aggttttgataatggcaggc

SEQ ID NO: 109 - DNA, 24 bp, MG979
actggactgtgcctacccactatg

SEQ ID NO: 110 - DNA, 20 bp, MG984
cctgtgtgaggatgagctct

SEQ ID NO: 111 - DNA, 16 bp, R302W
ttctcccgaagctcag

SEQ ID NO: 112 - DNA, 17 bp, R1236H
tttcttccgtgtgatga

SEQ ID NO: 113 - DNA, 33 bp, DF4EcoRI; identical to SEQ ID NO: 95

SEQ ID NO: 114 - DNA, 35 bp, DR816Xh; identical to SEQ ID NO: 96

SEQ ID NO: 115 - DNA, 20 bp, MG914
ctagcacagaactctgctgc

SEQ ID NO: 116 - DNA, 20 bp, MG837
ctagcacagaactctgctgc

SEQ ID NO: 117 - DNA, 24 bp, MG746
cttcagcaacagcgaagtagtctg

SEQ ID NO: 118 - DNA, 24 bp, MG747
gattctcagcacttgaaaagcagg

SEQ ID NO: 119 - DNA, 21 bp, MG773
ggacacatcagttttcctctc

SEQ ID NO: 120 - DNA, 20 bp, MG789
gaaaacccatgattcagtcc

SEQ ID NO: 121 - DNA, 20 bp, MG816
tcatcaggcaagaaacttgg

SEQ ID NO: 122 - DNA, 20 bp, MG803
gaagttggcaaaacagactg

SEQ ID NO: 123 - DNA, 20 bp, MG804
gagccatctgctcatttctg

SEQ ID NO: 124 - DNA, 20 bp, MG812
cccgctatttagacttgagc

SEQ ID NO: 125 - DNA, 21 bp, MG775
caaagtgtttattccaggagc

SEQ ID NO: 126 - DNA, 22 bp, MG802
catcagggtactttgaacattc

SEQ ID NO: 127 - DNA, 22 bp, MG727
ttgaccagaaaggctcagttcc

SEQ ID NO: 128 - DNA, 23 bp, MG915
agatgatgccagagggtttatcc

SEQ ID NO: 129 - DNA, 20 bp, MG790
tgcccagctctgttcaaacc

SEQ ID NO: 130 - DNA, 20 bp, MG774
aggcaatgactgactgacac

SEQ ID NO: 131 - DNA, 23 bp, MG805
tgcccgtctatttttgatgaagc

SEQ ID NO: 132 - DNA, 21 bp, MG791
tctcagttagtctggggacag

SEQ ID NO: 133 - DNA, 25 bp, MG751
tcatggtagagagactggactgtgc

SEQ ID NO: 134 - DNA, 21 bp, MG972
accctggagcaaatgacaacc

SEQ ID NO: 135 - DNA, 21 bp, MG973
atttgctccagggtacatggc

SEQ ID NO: 136 - DNA, 22 bp, MG974
gaaagacagtgggaaggcaagc

SEQ ID NO: 137 - DNA, 23 bp, MG975
gggagtgtgtggaacaaatgagc

SEQ ID NO: 138 - DNA, 25 bp, MG976
agtttctacaggctggtcctattcc

SEQ ID NO: 139 - DNA, 21 bp, MG755
aacgtggaatcccattgatgc

SEQ ID NO: 140 - DNA, 20 bp, MG730
tttctgtgttccctccttgc

SEQ ID NO: 141 - DNA, 20 bp, MG794
gatggtcaagttacactggc

SEQ ID NO: 142 - DNA, 24 bp, MG778
cacctcccaccaattatagtattc

SEQ ID NO: 143 - DNA, 23 bp, MG808
ctatgtgtgtctcttttacaggg

SEQ ID NO: 144 - DNA, 20 bp, MG817
aatctttcccaccatattgc

SEQ ID NO: 145 - DNA, 20 bp, MG779
cataccttcttttgctgtgc

SEQ ID NO: 146 - DNA, 23 bp, MG795
ccacagaagtcagaatctccacg

SEQ ID NO: 147 - DNA, 20 bp, MG731
tgtaacaaacctgcacgttg

SEQ ID NO: 148 - DNA, 23 bp, MG732
tgctacccaagccagtagtttcc

SEQ ID NO: 149 - DNA, 22 bp, MG788
gagtttgggaaagattggcagc

SEQ ID NO: 150 - DNA, 22 bp, MG772
tgtagtaaagcagctctcatgc

SEQ ID NO: 151 - DNA, 20 bp, MG733
caagtacactctgcactgcc

SEQ ID NO: 152 - DNA, 23 bp, MG758
tgactcaacttccccaccaagag

SEQ ID NO: 153 - DNA, 24 bp, MG736
ctccctatgtacgtggagtaatac

SEQ ID NO: 154 - DNA, 21 bp, MG737
gggagtcttgtgggaactaag

SEQ ID NO: 155 - DNA, 23 bp, MG780
ttcatagacatctctcagctctg

SEQ ID NO: 156 - DNA, 20 bp, MG759
gttttggtatcagggaaagc

SEQ ID NO: 157 - DNA, 20 bp, MG760
agccatgcttggaattttgg

SEQ ID NO: 158 - DNA, 20 bp, MG781
ctcactgggatgtcacaaac

SEQ ID NO: 159 - DNA, 25 bp, MG740
ggtcttgatgtgtgacttgtatccc

SEQ ID NO: 160 - DNA, 25 bp, MG741
cctcagtgtcacagtgttctttgtg

SEQ ID NO: 161 - DNA, 22 bp, MG809
catgaaatgactaggacattcc

SEQ ID NO: 162 - DNA, 20 bp, MG797
ctacccagtgacccaaacac

SEQ ID NO: 163 - DNA, 23 bp, MG761
cgaacccttagtttctgagacgc

SEQ ID NO: 164 - DNA, 20 bp, MG742
tcagtgccttggtgactgtc

SEQ ID NO: 165 - DNA, 21 bp, MG916
ttgatggtacagactggaggc

SEQ ID NO: 166 - DNA, 23 bp, MG810
aagaaagttgccaatcctgttcc

SEQ ID NO: 167 - DNA, 20 bp, MG762
agcacctgaaaataaggagg

SEQ ID NO: 168 - DNA, 22 bp, MG743
gcccaaagtttgtaagtgtgag

SEQ ID NO: 169 - DNA, 21 bp, MG787
agcaagaatgaggtcaagttc

SEQ ID NO: 170 - DNA, 24 bp, MG806
gggaaaaactggaggaaagaactc

SEQ ID NO: 171 - DNA, 21 bp, MG818
agaggtagggaaggaagctac

SEQ ID NO: 172 - DNA, 20 bp, MG813
ccaaagtccacttcttgaag

SEQ ID NO: 173 - DNA, 20 bp, MG834
gatgcactggttgctacatc

SEQ ID NO: 174 - DNA, 20 bp, MG836
ccaggacacttggtttctgc

SEQ ID NO: 175 - DNA, 20 bp, MG839
acactcccagttggaatcag

SEQ ID NO: 176 - DNA, 20 bp, MG871
cttgtgggcaagaaattgag

SEQ ID NO: 177 - DNA, 20 bp, MG829
tgggctggatgagactattc

SEQ ID NO: 178 - DNA, 24 bp, MG870
ccaaggacatatcttctgagcaac

SEQ ID NO: 179 - DNA, 20 bp, MG820
tgattatcagcataggctgg

SEQ ID NO: 180 - DNA, 20 bp, MG811
gatcccccaatagcaactgc

SEQ ID NO: 181 - DNA, 21 bp, MG763
cattcagattcaccaggacac

SEQ ID NO: 182 - DNA, 20 bp, MG782
ccttacatgccatctgatgc

SEQ ID NO: 183 - DNA, 20 bp, MG764
aaccttctcccctattaccc

SEQ ID NO: 184 - DNA, 22 bp, MG835
ggaaaatgagaggctataatgc

SEQ ID NO: 185 - DNA, 24 bp, MG1006
tgtattccagaggtcacccagagc

SEQ ID NO: 186 - DNA, 22 bp, MG1005
ccagtaagaaaggcaaacagcg

SEQ ID NO: 187 - DNA, 1094 bp, FANCD-S.ORF.s
ggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggtgcttcatcacctgtgtgggcattccaagattcaccaggacacgagactcacccaacatgtgcctctgctcaaaaagaccctggaacttttagtttgcagagtcaaagctatgctcactctcaacaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggatggtgaagaagacgaagtaagtgctggagaaaaggagcaagatagtgatgagagttatgatgactctgattagaccccagataaattgttgcctgcttctgtgtctcaa

SEQ ID NO: 188 - DNA, 1115 bp, FANCD cDNA OR
ggagaacacagccagcctttggaggaactactcagccagagcgtccattacttgcagaatttccatcaaagcattcccagtttccagtgtgctctttatctcatcagacttttgatggttattttggagaaatcaacagcttctgctcagaacaaagaaaaaattgcttcccttgccagacaattcctctgtcgggtgtggccaagtggggataaagagaagagcaacatctctaatgaccagctccatgctctgctctgtatctacctggagcacacagagagcattctgaaggccatagaggagattgctggtgttggtgtcccagaactgatcaactctcctaaagatgcatcttcctccacattccctacactgaccaggcatacttttgttgttttcttccgtgtgatgatggctgaactagagaagacggtgaaaaaaattgagcctggcacagcagcagactcgcagcagattcatgaagagaaactcctctactggaacatggctgttcgagacttcagtatcctcatcaacttgataaaggtatttgatagtcatcctgttctgcatgtatgtttgaagtatgggcgtctctttgtggaagcatttctgaagcaatgtatgccgctcctagacttcagttttagaaaacaccgggaagatgttctgagcttactggaaaccttccagttggacacaaggtgcttcatcacctgtgtgggcattccaagattcaccaggacacgagactcacccaacatgtgcctctgctcaaaaagaccctggaacttttagtttgcagagtcaaagctatgctcactctcaacaattgtagagaggctttctggctgggcaatctaaaaaaccgggacttgcagggtgaagagattaagtcccaaaattcccaggagagcacagcagatgagagtgaggatgacatgtcatcccaggcctccaagagcaaagccactgaggtatctctacaaaacccaccagagtctggcactgatggttgcattttgttaattgttctaagttggtggagcagaactttgcctacttatgtttattgtcaaatgcttctatgcccatttccattccctccataa

SEQ ID NO: 189 - DNA, 52 bp, Wild-type
ggaagccaggtgtggagaggaggcatggaatcttgctgaaattcagtctgtc

SEQ ID NO: 190 - DNA, 52 bp, Maternal Mutation
ggaagccgggtgtggagaggaggcatggaatcttgctgaaattcagtctgtc

SEQ ID NO: 191 - DNA, 52 bp, Maternal Mutation and Reversion
ggaagccgggtgtgaagaggaggcatggaatcttgctgaaattcagtctgtc